(19) United States
(12) Patent Application Publication
(10) Pub. No.: US 2007/0156021 A1
(43) Pub. Date: Jul. 5, 2007

(54) Remote imaging apparatus having an adaptive lens

(76) Inventors: Bradford Morse, Syracuse, NY (US); Thomas W. Karpen, Skaneateles, NY (US); Dongmin Yang, Syracuse, NY (US); Allan I. Krauter, Skaneateles, NY (US); Ervin Golfain, Syracuse, NY (US); Raymond A. Lia, Auburn, NY (US); Richard W. Newman, Auburn, NY (US); William H. Havens, Syracuse, NY (US); Ynjiun P. Wang, Cupertino, CA (US); Vivian L. Hunter, Baldwinsville, NY (US)

Correspondence Address: MARJAMA & BILINSKI LLP, 250 SOUTH CLINTON STREET, SUITE 300, SYRACUSE, NY, 13202, US

(21) Appl. No.: 11/521,172

(22) Filed: Sep. 14, 2006

Related U.S. Application Data
(60) Provisional application No. 60/717,583, filed on Sep. 14, 2005.

(51) Int. Cl. A61B 1/00; A61B 1/06

(52) U.S. Cl. 600/167; 600/176; 600/118

(57) ABSTRACT

Systems and methods for making and using endoscopes comprising one or more fluid lenses. An endoscope or bore-scope for various highly accurate imaging, visual inspection and measurement applications is equipped with an adaptive lens operated based on the electrowetting or electro-capillarity phenomenon. The adaptive lens is digitally controlled and is able to provide auto-focusing and optical zooming functions while remaining in a stationary position relative to a distal end section of the endoscope. Endoscopes equipped with these adaptive lenses provide a simpler, more compact design and a faster response while providing high quality images. Several functions needed in a variety of endoscopic imaging, inspection and measurement applications are further enhanced through the addition of a number of improvements of the adaptive lens itself and of the systems incorporating the adaptive lens.

### CROSS-REFERENCE TO RELATED APPLICATIONS

This application claims priority to and the benefit of co-pending U.S. provisional patent application Ser. No. 60/717,583, filed Sep. 14, 2005, which application is incorporated herein by reference in its entirety. The disclosures of the present application and of the above-identified application describe subject matter that has been invented by one or more employees of at least one of Welch Allyn, Inc., GE IT, Inc., and Hand Held Products, Inc., working under a written joint development agreement among those three entities that was in effect on or before the date the invention was made, and the disclosed invention was made as a result of activities undertaken within the scope of the joint development agreement. This application is also related to U.S. patent application Ser. No. 10/768,761, filed Jan. 29, 2004, entitled “Remote Video Inspection System,” and published as U.S. Patent Application Publication No. 20050129108 A1 on Jun. 16, 2005, which application is incorporated herein by reference in its entirety.

### FIELD OF THE INVENTION

The present invention relates to endoscopes or bore-scopes and more specifically to endoscopes and bore-scopes and to methods of using endoscopes or bore-scopes for imaging, visual inspection and measurement application where the endoscopes or bore-scopes incorporate various adaptive optical components.

### BACKGROUND OF THE INVENTION

In brief, a fluid lens comprises an interface between two fluids having dissimilar optical indices. The shape of the interface can be changed by the application of external forces so that light passing across the interface can be directed to propagate in desired directions. As a result, the optical characteristics of a fluid lens, such as whether the lens operates as a diverging lens or as a converging lens, and its focal length, can be changed in response to the applied forces.

Fluid lens technology that employs electrical signals to control the operation of the fluid lens has been described variously in U.S. Pat. Nos. 2,062,468 to Matz, 6,399,954 to Berge et al., 6,449,081 to Onuki et al., 6,702,483 to Tsuboi et al., and 6,806,988 to Onuki et al., in U.S. Patent Application Publication Nos. 2004/0218283 by Nagaoka et al., 2004/0228003 by Takeyama et al., and 2005/0002113 by Berge, as well as in several international patent documents including WO 99/18546, WO 00/58763 and WO 03/069380, the disclosure of each of which is incorporated herein by reference in its entirety.

Additional methods of controlling the operation of fluid lenses include the use of liquid crystal material (U.S. Pat. No. 6,437,925 to Nishioka), the application of pressure (U.S. Pat. No. 6,081,388 to Widl), the use of elastomeric materials in reconfigurable lenses (U.S. Pat. No. 4,514,048 to Rogers), and the uses of micro-electromechanical systems (also known by the acronym “MEMS”) (U.S. Pat. No. 6,747,806 to Gelbart), the disclosure of each of which is incorporated herein by reference in its entirety.

General Background Regarding Endoscopes

Endoscopes or bore-scopes have been used for imaging, visual inspection and measurement in medical and industrial applications where the access to an area of interest or to an object is limited due to location and or dimensional constraints.

Endoscopes have been designed and used for very specific applications such as to inspect aircraft engines, power plants and in other industrial environments. Reference is made in this regard to U.S. Pat. No. 3,778,170 to Howell assigned to the assignee of the instant invention (GE), U.S. Pat. No. 5,305,356 to Brooks, U.S. Pat. No. 6,529,620 to Thomson (assigned to Pinotage).

Endoscopes may have a rigid insertion tube or a flexible insertion tube.

Endoscopes may have an optical viewing system or an electronic display system. In the endoscopes having an electronic display, this display can be attached to the handset.

Some endoscopes have a handset including an insertion tube, where the handset is linked to an optical source and an electrical power source that are located into separate enclosures.

There are some portable endoscopes in which the handset includes the electrical power source to keep the electronic imager and the light source operating.

Endoscopes for various applications are equipped with stereo optical and stereo imaging components.

Most endoscopes have an optical system located at the distal end that includes various optical components that are all made out of high quality optical glasses.

In some of these “all glass endoscopes” a sapphire lens or several sapphire lenses are used to further improve the image quality and the overall optical performance.

Some endoscopes have been equipped with an additional fluid lens, so that the optical system is made of both glass and fluid lenses. This fluid lens is supposed to provide certain advantages and certain image quality improvements.

General Background Regarding Adaptive Optical Components

Improvements of the so called adaptive and/or variable shape lenses and mirrors have been applied to various devices such as in cellular phone cameras and more recently to endoscopes.

The electro-capillarity or electro-wetting phenomenon and its application has been reviewed by several authors and reference is made in this regard to the articles by B. Berge et al (Eur. Phys. J. E 3, 2000), S. Kuiper et al (Appl. Phys. Letters Vol. 85, N. 7, 2004 and F. Mugele et. al (J. Phys.: Condens. Matter 2005).

It is known to use an adaptive lens based on the electro-wetting phenomenon in various devices such as to perform auto-focus function.

It is also known to use an adaptive lens based on the electro-wetting phenomenon in various devices such as to perform optical zooming function.

General Background Regarding Endoscopes with Adaptive Optical Components

It is known to use variable shape or adaptive optical components not based on the electro-wetting phenomenon in endoscopes. Reference is made in this regard to U.S. Pat. No. 5,150,234 to Takahashi and to U.S. Pat. No. 6,437,925 Nishioka.

It is known to use variable shape or adaptive optical components based on the electro-wetting phenomenon in an endoscope. Reference is made in this regard to U.S. Pat. No. 6,934,090 to Nagaoka where an adaptive lens based on the electro-wetting phenomenon is used in an endoscope. As mentioned in FIG. 15 of Nagaoka '090 “it is possible to utilize the present variable optical element in an object optical system 72 in an endoscope 49. Here, the endoscope optical system 72 is disposed in the front most of the endoscope 49 so as to include the variable optical element 10. If a direction of the object optical system 2 can be designated desirably, it is possible to restrict the amount of the wave front aberration to be a predetermined amount or fewer by adjusting the rotational angle around the optical axis A such that the marks 2 and 3 should be disposed at predetermined positions”. No reference is made in Nagaoka '090 to the benefits of using an adaptive lens in an endoscope. No reference is made in Nagaoka '090 in regard to the problems faced by the known endoscopes and how these problems are solved by an adaptive lens based on the use of his endoscope 49.

There is a need to improve the current endoscopes using non-adaptive optical components that have auto-focusing and or optical zooming function based on the mechanical movement of the optical components.

There is a need to improve the current endoscopes using adaptive optics components for applications that require higher image quality, to make the optical systems more compact, and to achieve a higher quality image using a compact optical system.

### SUMMARY OF THE INVENTION

This invention teaches endoscopes or bore-scopes for imaging, visual inspection and measurement applications incorporating an optical system that includes one or more fluid lenses. As used herein, the term fluid lens is used to denote any fluid lens, including those based on the electro-wetting or electro-capillarity phenomenon, and those based on other methods for adjusting the behavior of a fluid lens, such as mechanical displacement of at least one surface of a fluid lens. One advantage of using an adjustable lens such as a fluid lens is that a user can have the benefit of multiple optical configurations in a single apparatus, which can eliminate the problems associated with having to remove an insertion tube merely to change the optical lens at the distal end of the insertion tube in order to attach a lens that is more suited to the visualization or to obtain an accurate measurement. Additional savings that are obtained by the use of an adjustable lens such as a fluid lens include the saving of time and effort to remove and reinsert the insertion tube, elimination of the possible difficulty in inserting the insertion tube to visualize the location that was examined prior to changing the lens, and avoidance of damage to the visualization apparatus and to the object being inspected or measured that can occur because of the added manipulation that changing a lens will involve.

In one aspect, the invention relates to a method of visualizing and measuring a remote object. The method comprises the steps of: providing an endoscope including a display, a hand set, an insertion tube, an optical system and an image sensor, wherein the optical system and the image sensor are located in a distal end of the insertion tube, said optical system comprising at least one adaptive lens; visualizing on said display at least a portion of a portion of a remote object by placing said insertion tube proximate said object; adjusting a focal length of said adaptive lens; and controlling automatically a focus of said adaptive lens by sequentially capturing a plurality of images of said object, storing said plurality of images in a memory buffer, and automatically selecting an optimum image for measurement based at least in part on an image quality criteria.

In one embodiment, said adaptive lens operates based on a selected one of an electro-wetting phenomenon and an electro-capillarity phenomenon. In one embodiment, said image quality criteria is a selected on of an edge contrast ratio, a MTF, and a surface roughness and a MTF. In one embodiment, the endoscope is stationary. In one embodiment, the entire endoscope is movable. In one embodiment, said image quality criteria includes a factor based on said variable lens and a factor based on said image sensor. In one embodiment, an illumination source is focused on an object to be inspected. In one embodiment, illumination from an illumination source is controlled as to match a field of view.

This invention teaches measurement endoscopes comprising an insertion tube having a distal end, an optical system and an imager located in the distal end, a fluid lens for performing an auto-focus function, where this fluid lens is placed: a) in the distal end or b) in a removal tip insert.

This invention teaches measurement endoscopes comprising an insertion tube having a distal end, an optical system and an imager located in the distal end, a combination of fluid lenses for performing: a) an optical zooming function; b) an auto-focusing and an optical zooming function, where these fluid lenses are placed: a) in the distal end, or b) in a removal tip insert or in both the removable tip and in the distal end.

This invention further teaches stereo measurement endoscopes comprising fluid lenses for either auto-focusing and or optical zooming function.

This invention further teaches visual inspection endoscopes where a fluid lens is used as a variable ND filter for imaging and or illumination purposes.

This invention further teaches visual inspection endoscopes where a fluid lens is used as a variable iris or stop in conjunction with optical systems comprising or not comprising fluid lenses for autofocus and/or optical zooming.

This invention teaches methods of using endoscopes or bore-scopes for imaging, visual inspection and measurement applications where the endoscopes or bore-scopes incorporate an optical system that includes fluid lenses based on the electro-wetting phenomenon.

This invention teaches improvements of the fluid lenses based on the electro-wetting phenomenon, where these adaptive lenses can be used in a variety of equipment and for many visual and illumination applications such as: a) in endoscopes or bore-scopes; b) medical devices; c) data and information reading scanners; other image capturing and visualization devices.

In one aspect, the invention relates to an adaptive lens for a remote imaging apparatus.

In another aspect, the invention features a remote imaging apparatus using an adaptive lens.

The foregoing and other objects, aspects, features, and advantages of an endoscope according to the invention will become more apparent from the following description and from the claims.

### BRIEF DESCRIPTION OF THE DRAWINGS

The objects and features of an endoscope according to the invention can be better understood with reference to the drawings described below, and the claims. The drawings are not necessarily to scale, emphasis instead generally being placed upon illustrating the principles of the invention. In the drawings, while every effort has been made to use like numerals to indicate like parts throughout the various views, given the number and complexity of the drawings, the right is reserved to make corrections should errors become apparent.

FIG. 1 corresponds to FIG. 1 of Matz, which was described therein as “a somewhat diagrammatical representation, partially in cross section, of [a fluid lens] apparatus” in which the direction of propagation of the beam is described by Matz as being upward, or parallel to the plane of the paper.

FIG. 2 corresponds to FIG. 2 of Matz, which was described therein as “a somewhat diagrammatical representation in elevation of a second modification of [a fluid lens apparatus] in which the direction of propagation of the beam acted upon is normal to the surface of the paper.”

FIG. 3 corresponds to FIG. 7 in Matz, which was described therein as “a diagrammatical representation of apparatus in combination with an optical device of the character described for biasing the device with a fixed electrical potential difference.”

FIG. 4 corresponds to FIG. 8 in Matz, which was described therein as “a somewhat diagrammatical representation of an optical system embodying the invention and comprising a liquid lens . . . and apparatus in conjunction therewith for utilizing the variance in vergency of the beam transmitted though the lens, showing such a system before an electric field has been impressed upon the lens, and where the transmitted beam has a maximum divergence.”

FIG. 5 corresponds to FIG. 9 in Matz, which was described therein as “a view similar to [FIG. 4] of the structure shown therein after a maximum electric field has been impressed upon the liquid lens and the divergency of the transmitted beam reduced to a minimum.”

FIG. 6 corresponds to FIG. 10 in Matz, which was described therein as “a cross-sectional view of a device embodying a modified form of [a fluid lens].”

FIG. 7 corresponds to FIG. 11 in Matz, which was described therein as “a somewhat diagrammatical representation in plan view of a further modification of [a fluid lens].”

FIG. 8 corresponds to FIG. 12 in Matz, which was described therein as “a cross-sectional view of a still further modification of [a fluid lens] wherein the electrodes are provided with beveled or inclined surfaces.”

FIG. 9 and FIGS. 9a-9g are an embodiment of an endoscope according to the invention showing an endoscope incorporating an adaptive lens based on the electro-wetting phenomenon located in the distal end of an insertion tube. The adaptive lens performs auto-focus, optical zooming and other functions.

FIG. 10 and FIGS. 10a-10f are another embodiment of an endoscope according to the invention.

FIG. 11 and FIG. 11a are another embodiment of an endoscope according to the invention.

FIGS. 12 and 12a-12e are another embodiment of an endoscope according to the invention.

FIGS. 13 and 13a-13j are another embodiment of an endoscope according to the invention.

FIG. 14a-b are another embodiment of an endoscope according to the invention.

FIG. 15a-e are another embodiment of an endoscope according to the invention.

FIG. 16a-c are another embodiment of an endoscope according to the invention.

FIG. 17a-b are another embodiment of an endoscope according to the invention.

FIG. 18a-d are another embodiment of an endoscope according to the invention.

FIG. 19a-b are another embodiment of an endoscope according to the invention.

FIG. 20a-c are another embodiment of an endoscope according to the invention.

FIG. 21 is a flow chart showing a process for operating a system having an adjustable focus system comprising focus acceptability feedback, according to principles of the invention.

FIG. 22 is a flow chart showing a process for operating a system having an adjustable focus system that does not comprise focus acceptability feedback, according to principles of the invention.

FIG. 23 is a circuit diagram showing a commutating power supply for a fluid lens system, according to principles of the invention.

FIG. 24 is a timing diagram showing a mode of operation of the commutating power supply of FIG. 23.

FIG. 25 is a flow chart of a calibration process useful for calibrating apparatus embodying features of the invention.

FIG. 26 is a diagram showing calibration curves for a plurality of exemplary endoscopes embodying features of the invention.

FIG. 27 is a diagram showing an embodiment of a power supply suitable for use with endoscopes according to principles of the invention.

FIG. 28 is a timing diagram illustrating an exemplary mode of operation of an endoscope according to principles of the invention.

FIG. 29a, FIG. 29b, and FIG. 29c are cross-sectional drawings showing an exemplary fluid lens with a mount comprising an elastomer for an endoscope according to principles of the invention.

FIG. 30 is a diagram illustrating a prior art variable angle prism.

FIG. 31 is a cross-sectional diagram of a prior art fluid lens that is described as operating using an electrowetting phenomenon.

FIG. 32a is a cross sectional diagram showing an embodiment of a fluid lens configured to allow adjustment of an optical axis, according to principles of the invention.

FIG. 32b is a plan schematic view of the same fluid lens, according to principles of the invention.

FIG. 33 is a schematic diagram showing the relationships between a fluid lens and various components that allow adjustment of the optical axis direction, according to principles of the invention.

FIG. 34a is a schematic diagram of an alternative embodiment of a fluid lens, according to principles of the invention.

FIG. 34b is a schematic diagram of an alternative embodiment of a distributor module, according to principles of the invention.

FIG. 35 is a schematic diagram showing the relationship between a fluid lens and a pair of angular velocity sensors, according to principles of the invention.

FIG. 36a through FIG. 36e are cross-sectional diagrams of another prior art fluid lens that can be adapted for use according to the principles of the invention.

FIG. 37 is a schematic block diagram showing an exemplary driver circuit.

FIG. 38A and FIG. 38 B are diagrams that show an LED die emitting energy in a forward direction through a fluid lens, according to principles of the invention.

FIG. 39A, FIG. 39B and FIG. 39C show diagrams of a laser scanner comprising a laser, a collimating lens, and a fluid lens in various configurations, according to principles of the invention.

FIG. 40 is a sketch of one embodiment of a zoom lens configuration, according to principles of the invention.

FIG. 41 is a diagram showing the zoom lens of FIG. 40 in more detail.

FIG. 42 is a diagram showing in greater detail the fluid lens elements of the zoom lens, according to principles of the invention.

FIG. 43 is a table that shows the detailed ZEMAX prescription for configuration 1 of a zoom lens comprising fluid lenses, according to principles of the invention.

FIG. 44 is a table that shows the detailed ZEMAX prescription for configuration 2 of a zoom lens comprising fluid lenses, according to principles of the invention.

FIG. 45 is a diagram showing the complete ray traces for the configuration 1 of a zoom lens comprising fluid lenses, according to principles of the invention.

FIG. 46 is a diagram showing the complete ray traces for the configuration 2 of a zoom lens comprising fluid lenses, according to principles of the invention.

FIG. 47 is a diagram showing the image spot sizes for configuration 1 of a zoom lens comprising fluid lenses, according to principles of the invention.

FIG. 48 is a diagram showing the image spot sizes for configuration 2 of a zoom lens comprising fluid lenses, according to principles of the invention.

FIG. 49 and FIG. 50 are diagrams showing prior art fluid lenses.

FIG. 51 is a diagram showing an illustrative variable aperture comprising a fluid lens.

### DETAILED DESCRIPTION OF THE INVENTION

The present application is directed to apparatus and methods useful for imaging, visual inspection and measurement applications incorporating an optical system that includes one or more fluid lenses. The apparatus and methods involve the use of one or more fluid lens components with endoscopes and borescopes to accomplish such tasks as imaging and inspection and measuring, including focusing on images of interest, and improving image quality by removing image artifacts.

U.S. Pat. Nos. 2,062,468 to Matz, 4,514,048 to Rogers, 6,081,388 to Widl, 6,369,954 to Berge et al., 6,437,925 to Nishioka, 6,449,081 to Onuki et al., 6,702,483 to Tsuboi et al., 6,747,806 to Gelbart, and 6,806,988 to Onuki et al., U.S. Patent Application Publication Nos. 2004/0218283 by Nagaoka et al., 2004/0228003 by Takeyama et al., and 2005/0002113 by Berge, and international patent publications WO 99/18456, WO 00/58763 and WO 03/069380 are each individually incorporated by reference herein in its entirety. The aforementioned published patent documents describe various embodiments and applications relating generally to fluid lens technology.

In the fluid lens technology of the present application, there are several different applications that can be applied generally to an apparatus, or used in a method. These include the following distinct inventions, which will be described in greater detail hereinbelow, and which can be applied individually or in combination in inventive devices:
1. in a device comprising a fluid lens, an image sensor, and a suitable memory, it is possible to record a plurality of frames that are observed using the fluid lens under one or more operating conditions, and to use or to display only a good or a most suitable frame of the plurality for further data manipulation, image processing, or for display; or alternatively, it is possible to use the plurality of frames as a range finding system by identifying which frame is closest to being in focus, and observing the corresponding focal length of the fluid lens;
2. in an apparatus comprising a fluid lens, additionally provide a temperature sensor with a feed back (or feed forward) control circuit, to provide correction to the fluid lens operating signal as the temperature of the fluid lens (or of its environment) is observed to change;
3. in a system comprising a fluid lens, additionally provide a non-adjustable lens component configured to correct one or more specific limitations or imperfections of the fluid lens, such as correcting color or aberrations of the fluid lens itself;
4. providing a calibration tool, process, or method for calibrating a fluid lens, for example involving operating the fluid lens at one or more known conditions (such as magnification), observing an operating parameter (such as driving voltage) at each known operating condition, saving the observed data in a memory, and using the data in memory to provide calibration data to be used when operating the fluid lens;
5. providing an inertial device such as an accelerometer to determine an orientation of a fluid lens, which orientation information is used to self-calibrate the fluid lens; and
6. in an apparatus comprising a fluid lens, operating the fluid lens to provide corrective properties with regard to such distortions as may be caused by vibration, location or orientation of the lens, chromatic aberration, distortions caused by higher order optical imperfections, and aberrations induced by environmental factors, such as changes in pressure.

In a very early fluid lens system, described by Matz in U.S. Pat. No. 2,062,468, now expired, a light transmitting liquid positioned between a plurality of electrodes operates as a lens of varying focal length or power. The variation of an intensity of an electrical potential impressed upon the liquid causes an alteration of a curvature of a surface of the liquid. Light passing through the liquid surface is caused to change intensity and/or vergence because of the shape of the liquid surface. The disclosure of Matz does not expressly identify the presence of a second fluid, such as air, that has an optical index different from that of the liquid, but claim 1 includes the recitation of “a light-transmitting dielectric liquid therebetween and exposed on one surface to another liquid of different refractive index, and interposed in the path of said beam.” It is apparent from the physics of transmission of light through optically transmissive media that only if a second fluid (such as air) is present would the light respond to the changing shape of the surface of the liquid described by Matz. The possibility of using a vacuum as the second medium is also recognized by the present inventors. However, Matz does not so much as hint at the use of vacuum. Since Matz says nothing about the environment of his fluid lens (e.g., nothing about operation in a specified ambient or container), one must conclude that the second fluid present in contact with the free surface of the liquid is room air.

Turning to the details of construction of the fluid lens, Matz describes a vessel that holds a light-transmitting low viscosity fluid of low electrical conductivity. The vessel can be an open tube or a vessel having a light transmitting end plate. As described by Matz, the device comprising an open tube or capillary structure can have a dual faced lens therein. Matz describes the dimension of an opening between electrodes as being small enough that the liquid surface can be shaped by surface tension and capillary action in the absence of an applied electric field. Matz describes electrodes made from various metals, but indicates that they can be made of any conductive material. In some embodiments described by Matz, the electrode faces are flat surfaces that face each other and define a slot or opening within which the liquid is situated. In other embodiments, the electrodes can be electrically conductive material coated on material such as glass. Matz also describes shaping the faces forming a slot in which the liquid is located, for example by making the faces curved or angularly positioned with respect to each other. In other embodiments, the electrodes can have curved surfaces, such as concentric annular structures.

Although Matz is incorporated by reference in its entirety herein, because Matz is a seminal description of fluid lens technology, certain portions of that disclosure and some of the figures presented therein are explicitly repeated herein in the following 19 paragraphs.

Matz states that his “invention contemplates primarily the use of a light-transmitting liquid positioned between a plurality of electrodes, as a lens of varying focal length or power, to alter the intensity or the vergency of a beam of light transmitted therethrough. The alteration in the intensity or vergency of the beam is effected by an alteration in the curvature of the surface of the liquid lens, which in turn is caused by an alteration in the intensity of the electric potential impressed upon the liquid between the electrodes.”

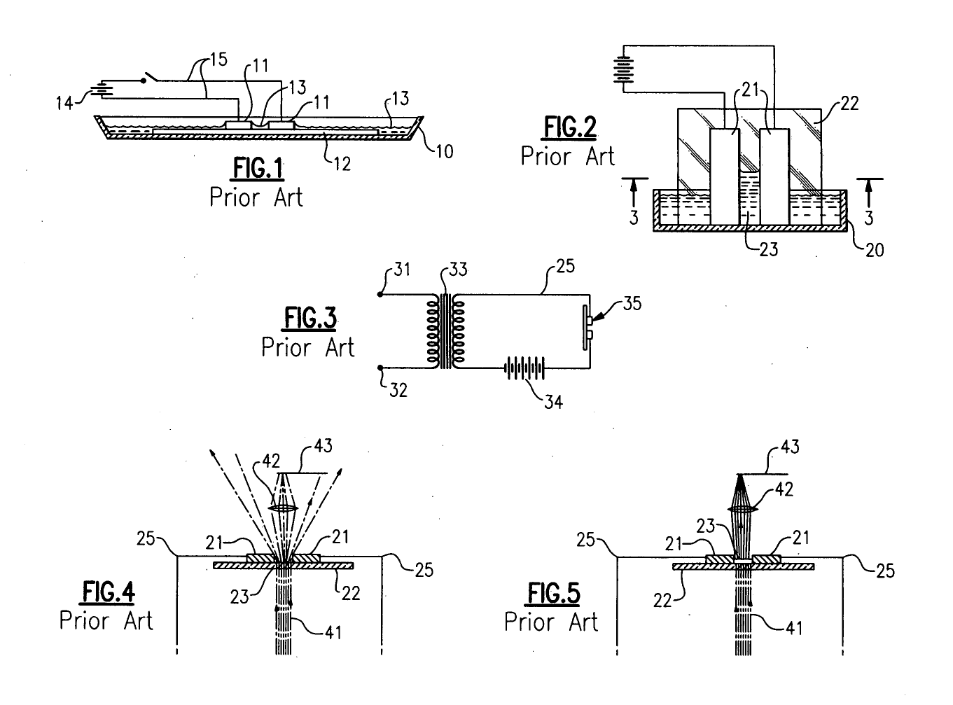

In FIG. 1 of the drawings one modification of the fluid lens is shown in which 10 represents any suitable container having a transparent base portion beneath the spaced electrode 11. The container may be of any suitable material, as for example glass. The electrodes 11 are preferably of any conducting material, as for example copper. brass, aluminum, or iron. They are positioned, as for example by fastening them either directly to the base of the container 10 or to a thin plate of glass 12, so as to provide a slot between the two electrodes. This slot should preferably be of such a width that a liquid 13 positioned therein between the electrodes presents an upper surface which is curved over its entire width. Preferably the slot is of such width only, however, as to permit the passage of an adequate beam of light, the electrodes being so closely placed as to permit the use of a relatively small potential difference. It has been found that if the electrodes are positioned so as to provide a slot approximately 0.020 inch in width the device will function admirably. The slot should preferably be of such depth as to permit full utilization of the curvature of the surface of the liquid 13 between the electrodes 11. For example, a slot having a width of 0.020 inch and a depth of one-eighth of an inch has been found satisfactory. It will be obvious that great variations in both the width and depth of the slot may be employed.

Means are provided, as for example a battery 14 and lead-in wires 15, for impressing an electrical potential difference between the electrodes 11 and across that portion of the liquid lying therebetween. Before the potential difference is impressed between the electrodes the liquid 13 is caused in general, by surface tension and capillary action, to present a concave surface, as shown for example, in FIG. Base1. If a parallel beam of light is projected upwardly through the device between the electrodes, this surface of the liquid acts as a negative lens to diverge the beam. If now a potential difference is impressed between the electrodes 11 and across the liquid lying therebetween, the effect upon the beam of light transmitted upwardly through the liquid is to decrease the degree of divergence depending upon the intensity of the impressed electric field to a point where the liquid lens acts substantially as a lens with zero power, so that the transmitted beam of light possesses the same characteristics as the incident beam.

For example, a device such as is shown in FIG. Base1, where the slot had a width of about 0.020 inch and where ethyl acetate was employed as the liquid forming the negative lens, with zero potential difference between the electrodes a beam of light passing through the lens was projected so as to form a band approximately two inches in width at a distance of two inches from the lens.

With an increase of potential difference the width of the transmitted beam decreased somewhat proportionally to the increase of potential until with a potential difference of about 500 volts the width of the transmitted band of light was only about one-eighth of an inch. In connection with the experiment just described the current employed was negligible, being probably only a few microamperes. The device described is therefore essentially an electrostatic instrument, and the power consumed by it is negligible.

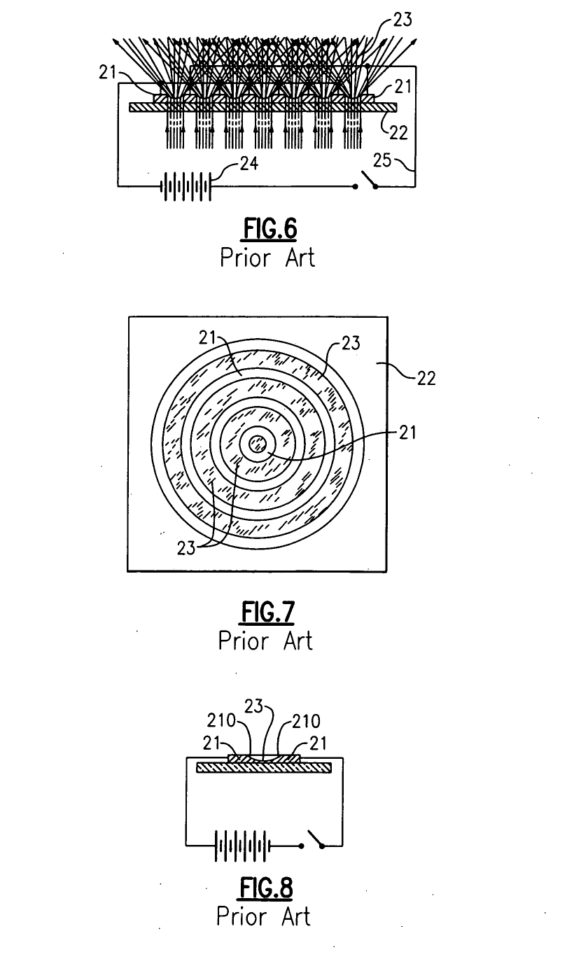

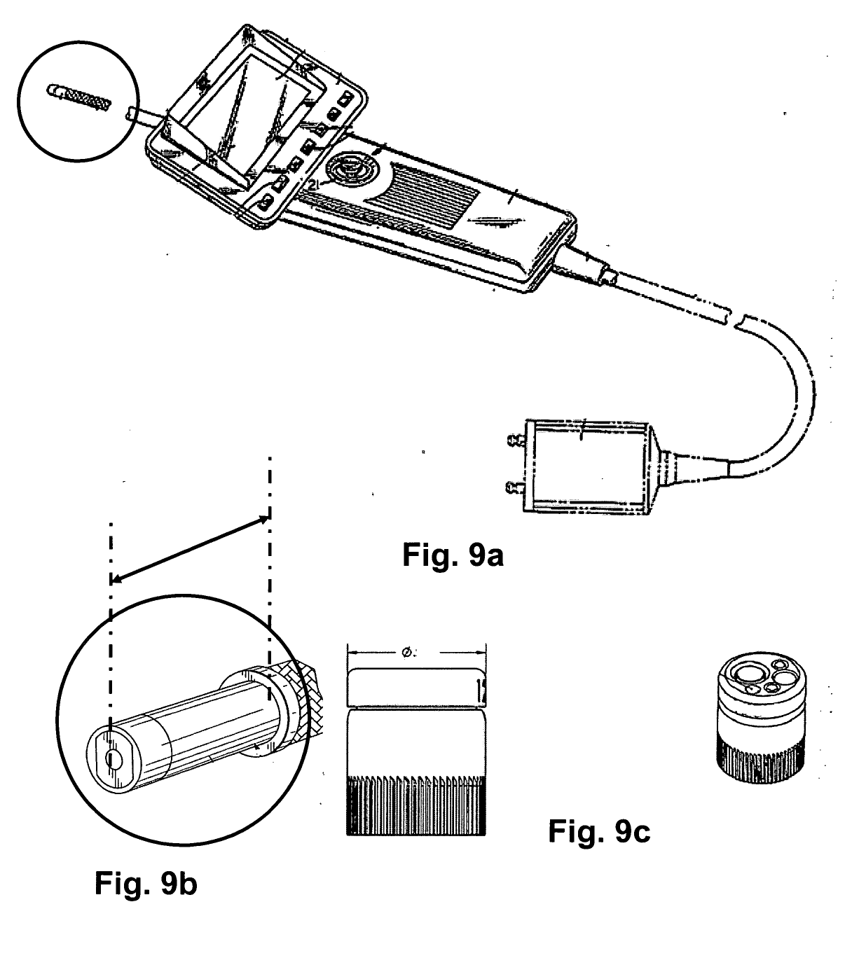

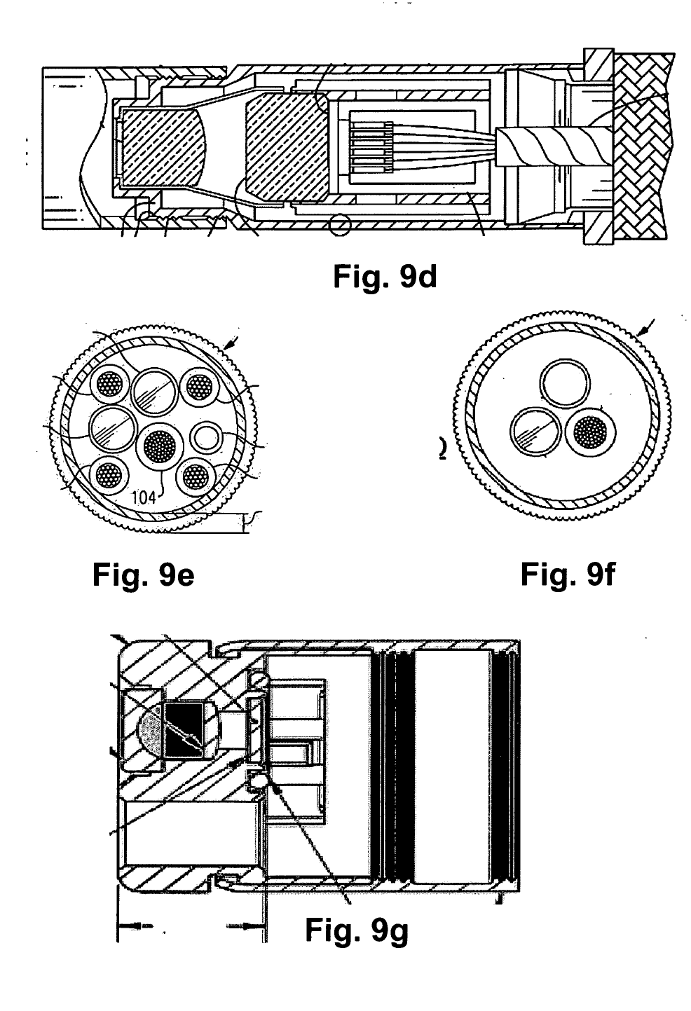

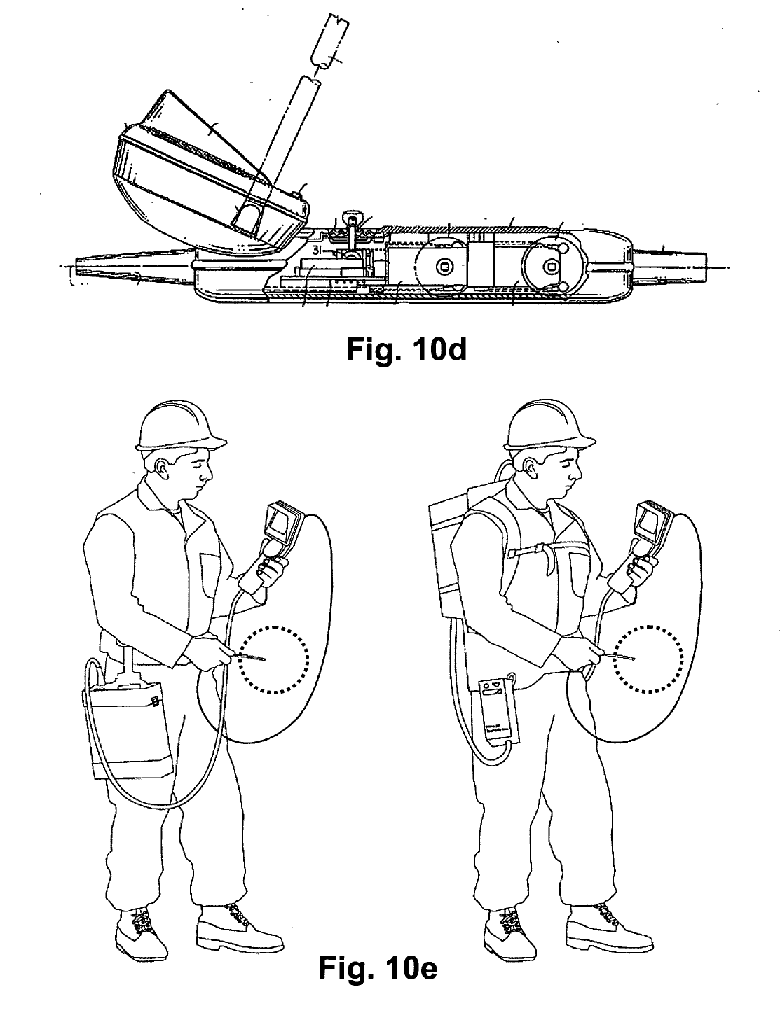

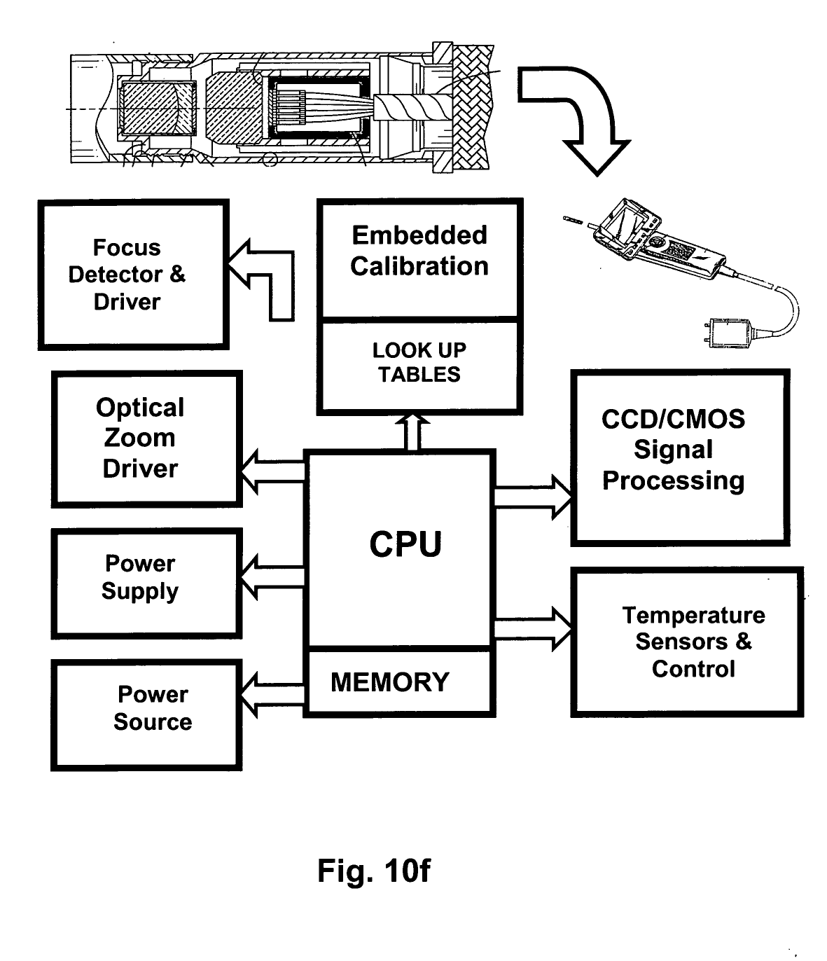

In FIG. 2 is shown a modification of the fluid lens in which the electrodes 21, with their supporting glass plate 22 forming a capillary channel, are mounted in an upright manner in any suitable container 20 instead of resting horizontally on the transparent base of the container, as shown in FIG. Base1. Where the device is used in this form the liquid 23, acting as a variable lens, is raised by the capillary action between the electrodes an appreciable distance above the surface of the liquid in the container. It is to be understood that the meniscus shown at the top of the column of liquid between the electrodes 21 in FIG. 2 is not the meniscus shown between the electrodes 11 of FIG. 1 or the electrodes 21 of FIG. 4 and FIG. Base5. The meniscus shown in FIG. 2 is merely that which is normally present at the top of a capillary column, and it is not employed primarily to act upon a transmitted beam. The meniscus which is employed to cause a vergence change in the transmitted beam is not shown in FIG. Base2, but is shown in FIGS. 4 and 5 (Matz FIGS. 8 and 9 respectively). In FIG. 4 (Matz FIG. 8) is shown a cross-sectional view of the device shown in FIG. 2 along the lines 3-3 and in a plane perpendicular to the plane of the drawings, i.e., a cross-section of the device shown in FIG. 2 taken at a point above the surface of the liquid in the container proper but below the upper end of the column of liquid between the electrodes.

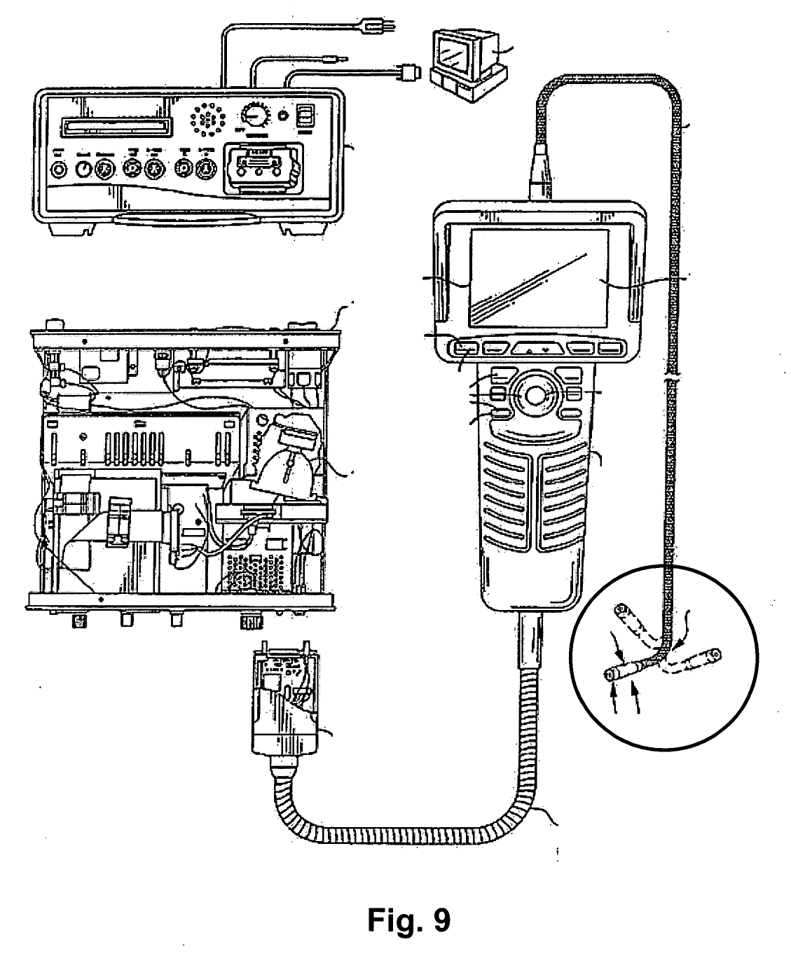

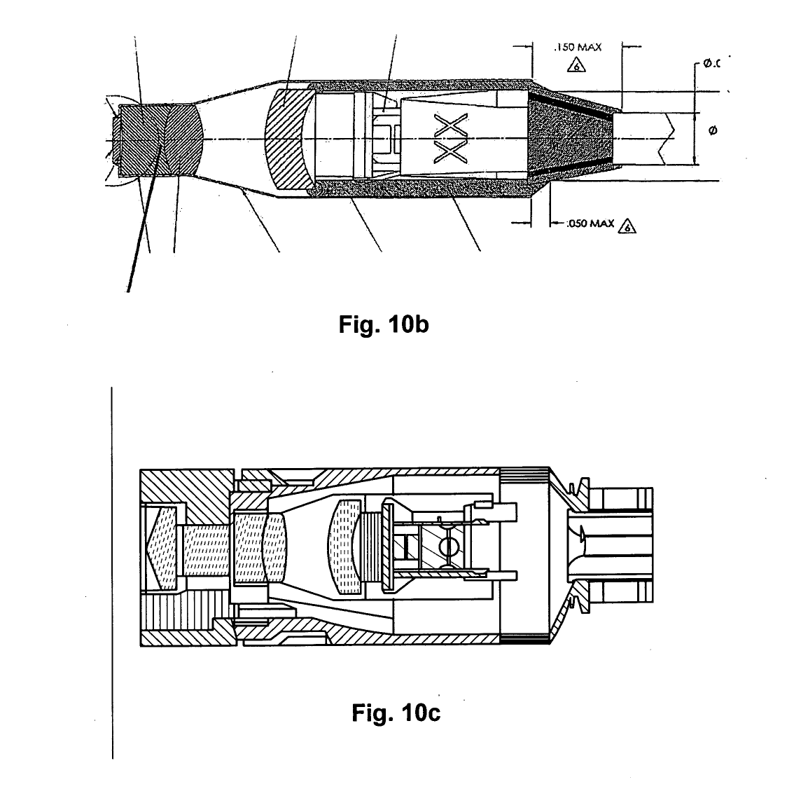

It has been found desirable at times to operate devices of the character described with a bias impressed upon the liquid lens. In FIG. 3 (Matz FIG. 7) a circuit is shown to effect this result in which 31 and 32 represent lead-in wires, 33 a transformer, and 34 a source of constant potential difference in circuit with the liquid lens 35 and adapted for impressing a constant bias upon the lens. With such a set-up alterations in the current in the lead-in wires give rise to induced alterations in the potential of the secondary circuit comprising the liquid lens, with the result that the lenticular characteristics of the lens are altered and its effect upon the transmitted beam changed. It will be obvious that many other standard methods of biasing may be employed with this new type of light valve.

In FIGS. 4 and 5 (Matz FIGS. 8 and 9) an optical system is disclosed illustrating one possible use of the new valve. In these drawings, the numeral 21 represents the conducting elements forming with their non-conducting, transparent, supporting plate 22 a capillary channel, within which the transparent, dielectric liquid 23 rises to act as a lens on the transmitted beams 41. Adjacent this liquid lens a suitable positive lens 42 may be positioned adapted to focus an image of the slit between the electrodes 21, or as shown, an image of the light source, on a recording film or other suitable surface 43. With such an apparatus, when the liquid lens is not subjected to an impressed electric field it acts as a negative lens to diverge the transmitted beams of light so that only a relatively small amount of the transmitted light falls upon the lens 42 and is focused thereby upon the recording film 43. The image of the light source thus made on the film is a faint image. As an electric potential is impressed upon the liquid lens and its lenticular characteristics altered, so that it assumes more nearly the characteristics of a lens of zero power, the divergence of the transmitted beam of light is reduced so that more and more light falls upon the lens 42 and is focused thereby upon the recording film 43, until a maximum condition is reached, as shown for example in FIG. 5 (Matz FIG. 9), where substantially all of the light transmitted though the liquid valve is focused upon the recording film. When this condition is reached the intensity of the image of the light source which is recorded on the film 43 is a maximum.

It will be understood also that substantially the same results are to be obtained if instead of a lens 42 interposed in the path of the transmitted beam and between the liquid lens and the recording strip, an opaque element is interposed with a slot in registry with the recording film and the slit between the electrodes 21. The light which passes through such a slot and which is recorded on the film will have a varying intensity, depending upon the condition of the liquid lens, which in turn, as has been pointed out, is a direct function of the intensity of the impressed potential thereon.

It will be understood also that the device may be employed to record a strip of varying width upon a suitable recording film. If for example the film 43 in FIGS. 4 and 5 (Matz FIGS. 8 and 9) is brought closely adjacent the liquid lens 23, and if the lens 42 is removed from the optical system, then the divergence of the beam transmitted by the liquid lens will be recorded directly upon the recording film, so that the record of alterations in the impressed potential across the liquid lens will be formed as an exposed strip of varying width upon the recording film. The device has been described as comprising a plurality of electrodes mounted upon a non-conducting transparent support with a fluid positioned between the electrodes and reacting to the impressment of an electric field so as to present an alternating surface curvature in the path of a transmitted beam of light. The device will function also if the supporting plate for the electrodes is omitted, in which case the fluid will rise between the electrodes by capillary action and will present a double lens face to a transmitted beam. It is thought, however, that the form shown in the drawings and described above, i.e., with the supporting glass plate, is to be preferred. If the double lens face of the liquid lens is desired, it may better be secured by using a single glass plate support with electrodes mounted on each face thereof so that two columns of liquid are provided.

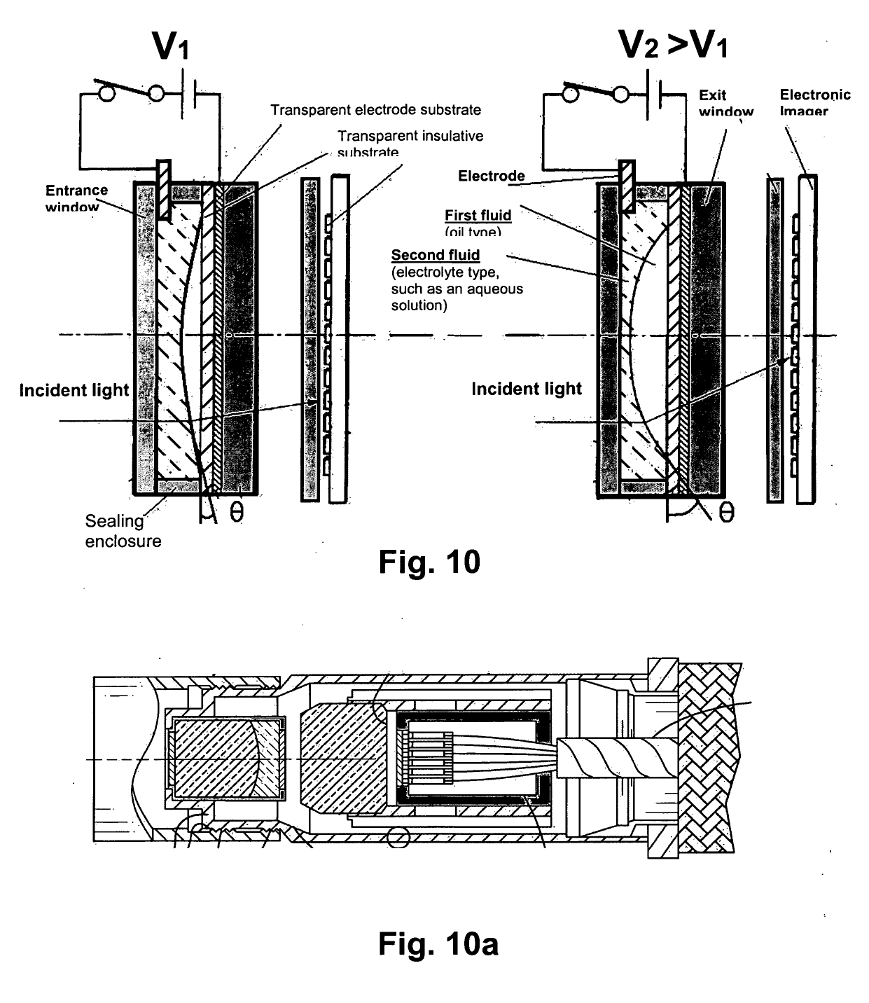

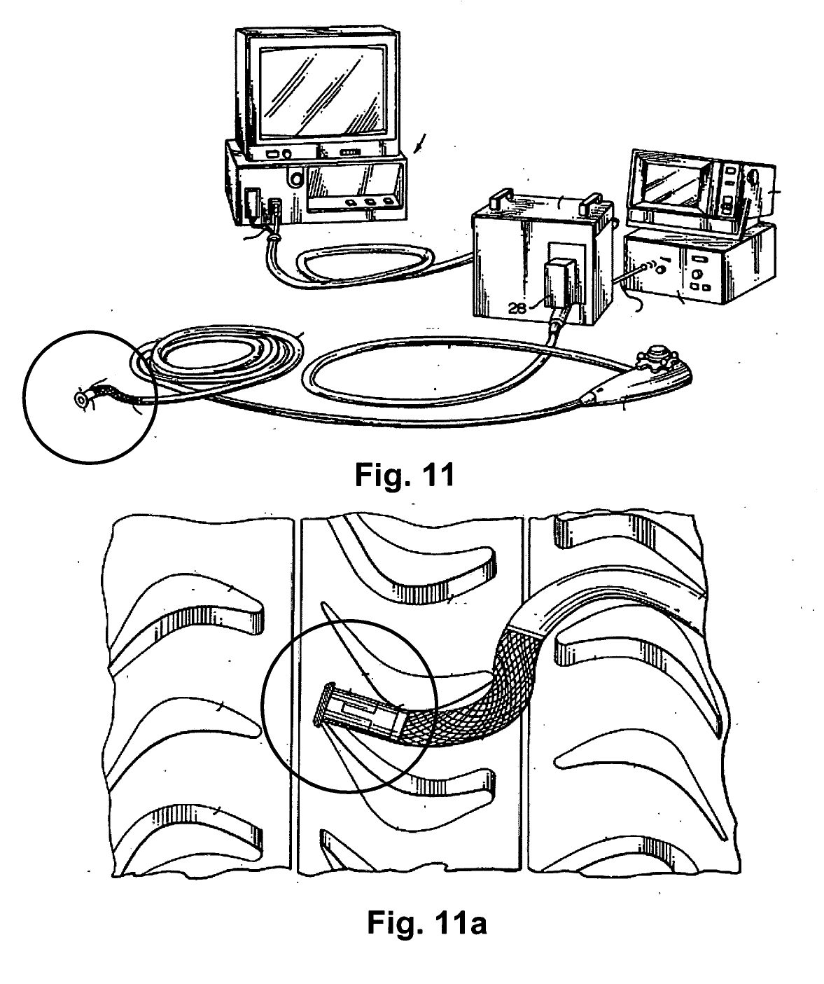

It will be obvious also that the lenticular effect may be secured if desired in a great variety of ways. For example, a plurality of slots may be employed so that beams passing therethrough may commingle in the dispersed condition and may be separated when a potential is impressed on the liquid lenses. Such a structure is shown, for example, in FIG. 6 (Matz FIG. 10), where 21 represents the electrodes, 22 the supporting glass plate, 23 the fluid between the electrodes, 24 a source of potential, and 25 conductors leading to the electrodes. As shown in the figure, the liquid lenses between adjacent pairs of electrodes are concave and the transmitted beam is scattered at each liquid lens. When a suitable supplementary lens is employed with such a device, i.e., a device using a multiplicity of liquid lenses, the transmitted beam when the field is not impressed on the liquid lenses, will be diffuse and cannot be bought to a focus at the focal point of the said lens. When, however, the field is impressed on such a device a plurality of substantially parallel intense beams are transmitted which may be brought to a focus at the focal point of the said lens.

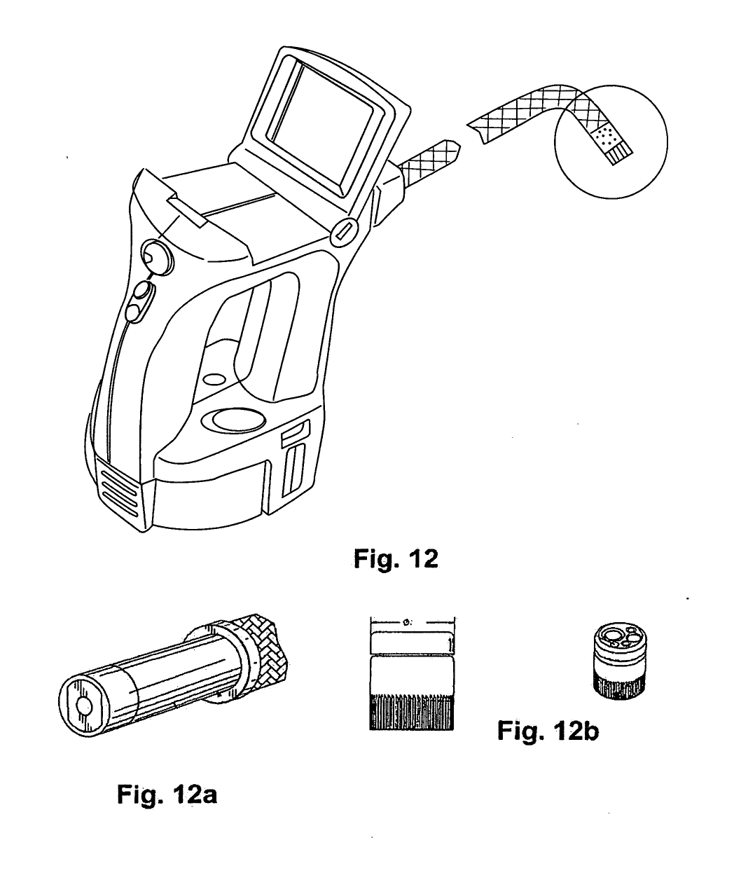

A plurality of ring-shaped electrodes may be employed with circular slots therebetween to secure the transmission of, for example, concentric beams, which may be diffuse and diverging or intense and substantially parallel depending upon the intensity of an impressed electric potential. Such a device is shown somewhat diagrammatically in plan in FIG. 7 (Matz FIG. 11), where 21 represents the electrodes and 23 the concentric circular capillary channels therebetween. In connection with this figure it is to be understood that the direction of the transmitted beam would be at right angles to the plane of the paper on which the figure appears. It will be obvious that any desired shape of electrodes may be employed.

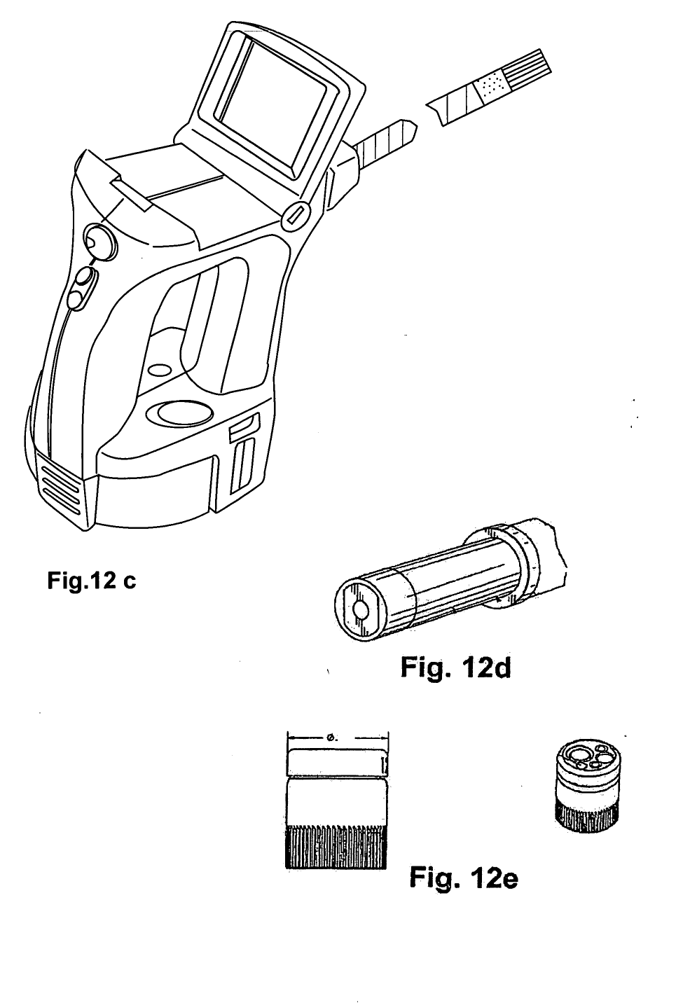

While the electrodes have been shown as provided with substantially perpendicular faces forming the side walls of the slot containing the liquid lens, it will be understood that electrodes of other shapes may be employed. For example, the faces forming the slot may be curved or angularly positioned with respect to each other. Such a device is shown in cross section in FIG. 8 (Matz FIG. 12), where the electrodes 21 are shown with inclined faces 210, which form the side walls of the capillary channel holding the liquid 23. It will be understood also that the electrodes may be small and the capillary action secured by other elements associated therewith. For example, in FIG. 2 the plates 21 which are shown as electrodes, may, if desired, be plates of other materials, as for example glass, coated with a conducting material to form electrodes along the sides of that portion of the slot which is employed to transmit light.

It will be understood also that while the depth of the slot has been described as more or less uncritical, provided it is of sufficient depth to permit adequate curvature of the surface of the material therein, it may be desired to employ a slot of such depth, and material within the slot of such depth, that the surface tension of the material causes the apex of the curvature of the surface to lie approximately upon the supporting glass plate so that at that region the fluid within the trough forms merely a film upon the plate.

While the operation of the device has been described as adaptable primarily to an alteration in the surface curvature of the liquid lens, it is to be understood that there are other associated effects which may contribute largely to the successful operation of the system, and may be important in the modulation of some frequencies. The electrocapillary rise and fall of the fluid in the slot where the device is employed, for example, as shown in FIG. Base2, may be employed to augment the modulating effect of the alteration in the lenticular structure of the fluid. This capillary rise and fall is, however, probably relatively slow, and where the device is used as a light valve with high frequencies, it probably has little effect.

Where a liquid is employed in the device which absorbs certain wave lengths of the transmitted beam, the device may be effective to alter the intensity of the beam because of the alteration in the effective thickness of the film of liquid interposed in the path of the beam at the center of the slot with the impressment of the electric potential.

The fluids employed in the valve are preferably light-transmitting, low-viscosity fluids of low electrical conductivity. For example, ethyl acetate is an excellent fluid. A wide variety of liquids have been found usable, however, such for example as methyl alcohol, ethyl alcohol, ether, carbon tetrachloride, methyl acetate, distilled water, glycerine, nitrobenzene, and some oils.

The device which bas been described and which has been termed a liquid lens of variable focal length has many other applications. It may be employed, for example, as an electrostatic voltmeter, as the alteration in the divergence or convergence of a translated beam is a function of the intensity of the impressed field. The device may be employed in connection with suitable apparatus for the transmission of audible or other signals over a beam of light. When the device is employed in connection with transmission of audible signs it may be said to modulate the beam of light at audible frequencies, and where such an expression is used in the claims it should be so interpreted. It is admirably adapted for use in sound-recording on motion picture film.

Claim 1 of Matz is also repeated as a description of a fluid lens: Means for modulating a light beam at audible frequencies comprising a plurality of elements forming a capillary channel having opposite electrically-conductive portions, a light-transmitting dielectric liquid therebetween and exposed on one surface to another liquid of different refractive index, and interposed in the path of said beam, and means to impress an electric potential on said liquid.

Although Matz describes his fluid lens as being responsive to “an electric potential,” it is clear that different fluid lens technologies can be used that respond to signals that are voltages (electric potentials, or electric potential differences), as well as signals that can be characterized by other electrical parameters, such as electric current or electric charge (the time integral of electric current). One can also design lenses that have adjustable behavior based on the interaction of light with two or more fluids (or a fluid and vacuum) having differing optical indices that operate in response to other applied signals, such as signals representing mechanical forces such as pressure (for example hydrodynamic pressure), signals representing mechanical forces such as tensile stress (such as may be used to drive elastomeric materials in reconfigurable lenses), and signals representing a combination of electrical and mechanical forces (such as may be used to drive micro-electromechanical systems). For the purposes of the present disclosure, the general term “fluid lens control signal” without more description will be used to denote an applied signal for driving any type of fluid (or reconfigurable) lens that responds to the applied signal by exhibiting adjustable behavior based on the interaction of light with two or more fluids (or a fluid and vacuum) having differing optical indices.

As is well understood in the optical arts, the distance at which apparatus according to the invention can operate, or equivalently, a focal length of the optical system of the apparatus, can vary as the distance q from the lens to the object to be imaged varies. The focal length for a specific geometrical situation can be determined from the formula
1/f=1/p+1/q
in which f is the focal length of a lens, p is the distance from the lens to a surface at which a desired image is observed (such as an imaging sensor or a photographic film), and q is a distance between the lens and the object being observed.

Consider the two objects situated at a nearer distance q₁ and a farther distance q₂ from the lens (e.g., q₂>q₁). In a system that is less expensive and more convenient to construct, the distance p (from the lens to the imaging sensor) is fixed. One can image objects lying at the distance q₁ from the lens with a focal length given by 1/f₁=1/p+1/q₁, and one can image objects lying at the distance q₂ from the lens with a focal length given by 1/f₂=1/p+1/q₂. Since q₂>q₁, and p is constant, we have f₁<f₂. In particular, for an endoscope comprising a fluid lens that can provide a minimum focal length of f₁ and a maximum focal length of f₂, for a fixed value of p, one would have the ability to observe in proper focus objects at distances ranging at least from q₁ to q₂, without consideration for issues such as depth of field at a particular focal length setting of the lens. By way of example, q₁ might be a short distance such as 4 inches (approximately 10 cm) so that one can image a target object having much detail with recovery of all of the detail present in the object. On the other hand, q₂ might be a longer distance, such as 12 inches (approximately 30 cm) or more, whereby an instrument can image an object at longer distance with lesser density (e.g., fewer pixels of resolution per unit of length or area observed at the target object). Accordingly, an endoscope of the invention comprising a particular imaging sensor can be configured to perform at either extreme of high density/short distance or of low density/long distance (or any variant intermediate to the two limits) by the simple expedient of controlling the focal length of the fluid lens such that an object at the intended distance d in the range q₂≧d≧q₁ will be imaged correctly.

The lens can be caused to either manually or automatically change its focal length until the best focus is achieved for an object at a given distance away. One way to do this is to minimize the so-called blur circle made by a point or object within the field of view. This can be done automatically by a microprocessor that varies the focal length of the lens and measures the size of the blur circle on a CCD or CMOS imager; i.e. the number of pixels the blur circle fills. The focal length at which the blur circle is smallest is the best focus and the lens is held at that position. If something in the field of view changes, e.g. the object gets farther away from the lens, then the microprocessor would detect the change and size of the blur circle and reinitiate the automatic focusing procedure.

The object used to measure the blur circle could be a detail inherently in the field of view, or it could be a superimposed object in the field of view. As an example, one could project an IR laser spot into the field (the wavelength of the IR is beyond the sensitivity of the human eye, but not of the CCD). Another means of achieving best focus includes transforming the CCD or CMOS image into the frequency domain and then adjusting the focal length of the fluid lens to maximize the resulting high frequency components of that transformed image. Wavelet transforms of the image can be used in a similar fashion. Both the frequency domain and wavelet techniques are simply means for achieving best focus via maximization of contrast among the pixels of the CCD or CMOS sensor. These and similar means, such as maximizing the intensity difference between adjacent pixels, are known in the art and are commonly used for passive focusing of digital cameras.

This invention teaches several endoscopes used for visual inspection. The endoscopes incorporate an adaptive lens based on the electro-wetting or electro-capillarity phenomenon to perform novel functions.

Adaptive lenses based on the electro-wetting phenomenon can use a single fluid, a gas, or two fluids having different optical and or chemical characteristics. The optical properties of these lenses can be changed by applying a voltage or an electrical current to alter among others the shape of the fluid, the fluids or the gas that forms the adaptive lens. This presents a practical advantage for optical systems that need to be placed in a tight space and that require less power to change the optical characteristics. These adaptive lenses and other components can be used for imaging applications to perform functions such as auto-focusing, optical zooming, and brightness adjustment.

Information about these types of adaptive lenses can be found in U.S. Pat. No. 6,368,954 to Berge, US Patent Application Publication 2005/0002113A1 to Berge, U.S. Pat. No. 6,538,823 to Kroupenkine, U.S. Pat. No. 6,545,816 to Kroupenkine, U.S. Pat. No. 6,702,483 to Tsuboi and U.S. Pat. No. 6,806,988 to Onuki, the disclosure of each of which is incorporated herein by reference in its entirety.

FIG. 9a through FIG. 9g show several embodiments of an endoscope or bore-scope system that includes a flexible insertion tube having a distal end located at a steering end of the insertion tube. Other components and the functionality of the endoscope can be understood from U.S. Pat. No. 5,373,317 assigned to the patentee of this invention, this patent being incorporated herein by reference.

As further shown in FIG. 9a to FIG. 9g. the distal end of the insertion tube has a fixed portion that includes an optical system made of lenses and an electronic imager device such as a CCD or CMOS sensor or imager, which can be a multipixel array. The operation of the endoscope system from an optical and electronic view point can be learned from U.S. Pat. No. 5,754,313 and U.S. Pat. No. 5,857,963 assigned to the patentee of the current invention, where these two patents are incorporated herein by reference.

As further shown in FIG. 9a to FIG. 9g, the optical system of the endoscope includes a removable optical tip that incorporates additional optical elements that can be removed and replaced based on specific needs. Cross sections through several portions of the insertion tube and of the distal end are shown in FIG. 9e and FIG. 9f. The end portion of the insertion tube can be adjusted from an angular view point via a steering mechanism incorporating four steering cables 141. To do so the insertion tube is flexible and has a higher flexibility at portion. A video cable is connected to the electronic imager. In the cross section of FIG. 9f one can see a fiber optics bundle that provides illumination and the optical system that provides the image of the object to be inspected to the electronic imager. The operation of the steering cables can be learned in more detail from U.S. Pat. No. 4,941,454 assigned to the patentee of the current invention, wherein this patent is incorporated herein by reference. Other steering mechanisms can be used such as shown in U.S. Pat. No. 4,794,912 assigned to the patentee of the current invention, wherein this patent is incorporated herein by reference.

As further shown in FIG. 9g the removable optical tip has a body portion that is attachable to the fixed end portion of the distal end of the endoscope. This tip usually includes an optical system that in conjunction with the optical system of the fixed portion of the distal end forms the image of the object under inspection.

According to some embodiments of this invention, the tip may incorporate optical components made of glass or other optical materials that are “passive” from an optical view point, or may incorporate a single or several adaptive optical components based on the electro-wetting phenomenon.

According to some embodiments of this invention, the fixed portion of the distal end may incorporate optical components made of glass or other optical materials that are “passive” from an optical view point, or may incorporate a single or several adaptive optical components based on the electro-wetting phenomenon.

According to this invention, at least a single or several adaptive optical components based on the electro-wetting phenomenon are used in the distal end of the insertion tube to perform auto-focusing, optical zooming, variable illumination and other optical functions, where unlike in the known endoscopes these functions are mostly done using movable optical components. Unlike in the optical systems using polarizing elements for endoscopes, such as in U.S. Pat. No. 5,150,234 to Takahashi, the current invention introduces the use of electro-wetting lenses to perform auto focusing and optical zooming without reducing the amount of the light which is needed in most endoscopic applications that require accurate measurements and good visibility of the object under investigation.

As shown in FIG. 9g, the optical system is formed of an auto-focusing fluid lens which has a sapphire lens attached to entrance portion of the fluid lens.

The operation of the fluid lens is shown from a schematic view point in FIG. 10 and details regarding the materials of the two fluids or the single fluid can be learned from the patents mentioned previously in regard to the electro-wetting phenomenon and its use in optical imaging applications.

More details regarding the optical system using fluid lenses in an endoscope are shown in the embodiments of FIG. 10a through FIG. 10c. The distal end of the endoscope of the current invention has a fluid lens made of a first fluid and a second fluid having different characteristics. The fluid lens is placed behind the removable tip and in front of the imager. This fluid lens is used to perform auto-focusing by applying a voltage and changing the shape of the interface between the two fluids. FIG. 10b shows another embodiment of an endoscope according to the invention where a zoom fluid lens is used at the entrance of the distal end fixed portion. This zoom fluid lens is made of three fluids, two being electrolytes and an insulating oil type fluid located in between them. This is a most compact design where there is no space between these elements and thus is useful for endoscopes of a compact design. More benefits are achieved by using lens as an entrance window for the CCD or CMOS sensor or imager.

In the embodiment of FIG. 10c a prism is used in the removable tip for a stereoscopic measurement application.

In the embodiment of FIG. 10d a handset is shown accommodating several commands related to the actuation of the distal end and the fluid lenses.

In the embodiment of FIG. 10e the operation of a portable endoscope using the fluid lenses shown in all the embodiments is shown, where the power source is attached to the person performing the inspection.

In the embodiment of FIG. 10f the blocks needed to operate the endoscope and the fluid lenses are depicted.

In the embodiments of FIG. 11 and FIG. 11a one can see other configurations of the endoscopic system using a fluid lens at the distal end.

In the embodiments of FIG. 12, and FIG. 12a through FIG. 12e other configurations of portable endoscopes using at least one fluid lens for autofocus and several lenses for optical zooming are shown.

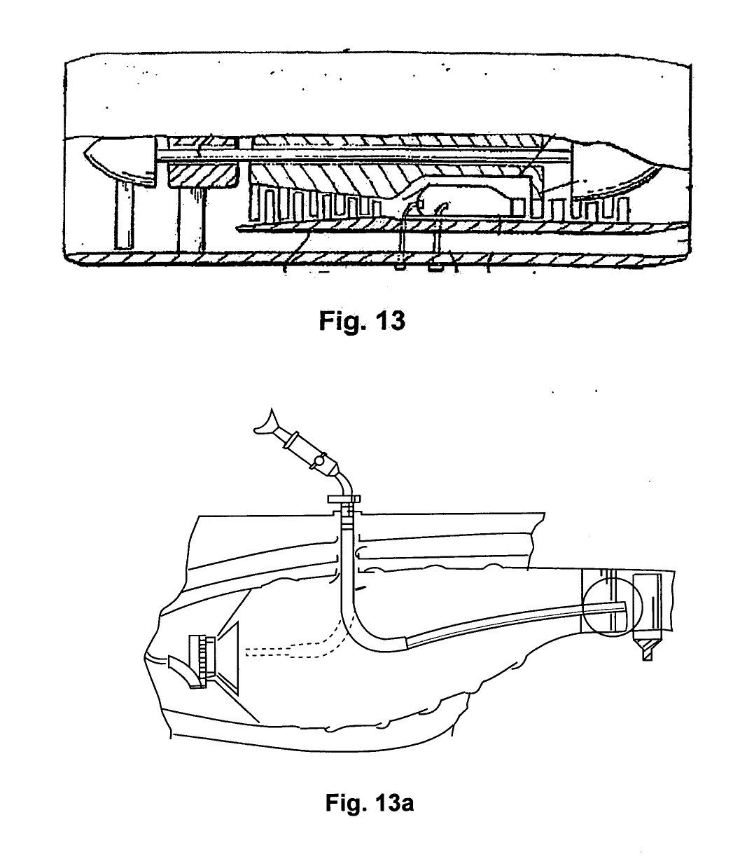

Methods of using the endoscope shown in the embodiments of the current invention are shown in the embodiments of FIG. 13, FIG. 13a through FIG. 13h for inspecting an aircraft engine having several blades that may have a damaged portion that needs to be detected and measured.

It is advantageous for such applications to have as perfect a focus of the optical system as is practical and in additional optical magnification in order to perform an accurate measurement (see FIG. 13i).

FIGS. 13j(a) and 13j(b) show measurements of surface texture including a power spectral density measurement for an object under inspection. For example, one may want to inspect coatings on turbine blades or in pipes, corrosion of materials subjected to aggressive fluids and/or heat and the like.

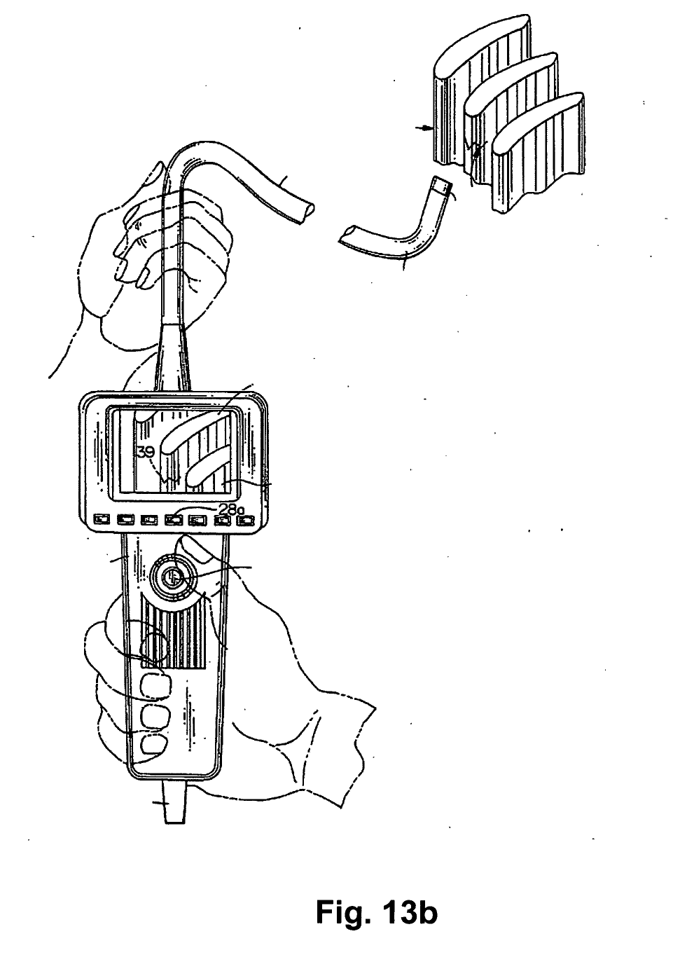

The auto-focus functions can be done in various ways. As shown in the embodiments of FIG. 14a and FIG. 14b the auto-focus is done by registering several frames of images and using the roughness information of the object to do a Modulation Transfer Function (“MTF”) analysis using random algorithms. (See Applied Optics Vol. 38, No, 4, 1999.) Once the best focused image is selected the measurement can be initiated.

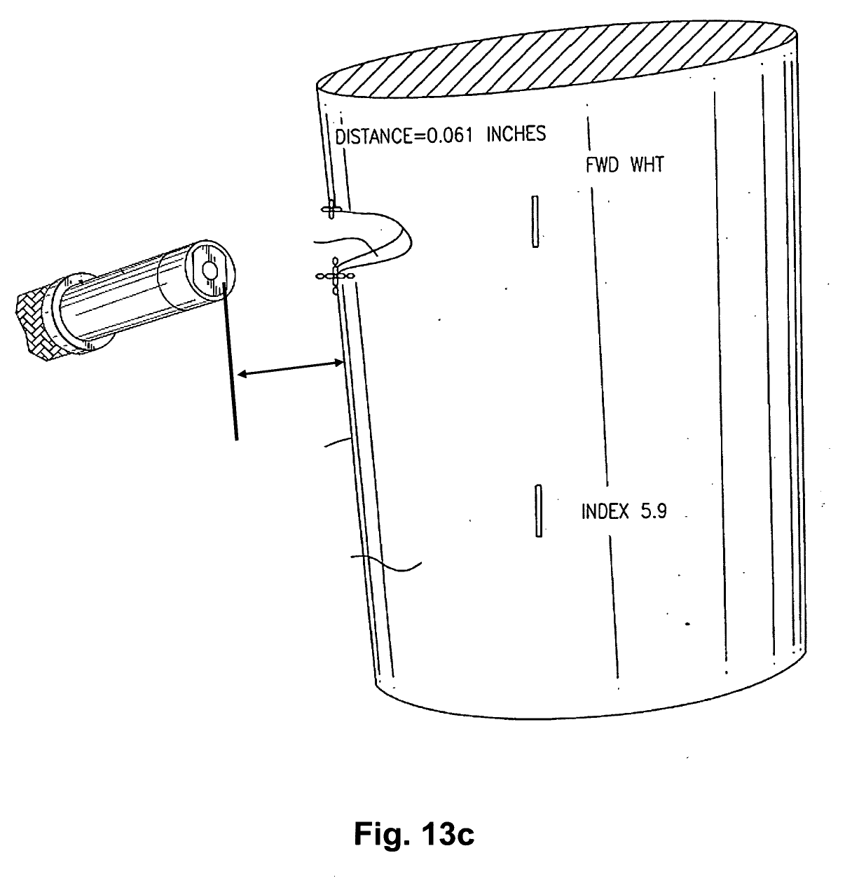

In the embodiments of FIG. 15a through FIG. 15c details are provided regarding the electrical operation of the fluid lens located in removable tips, but the same is valid if the fluid lenses are located in the fixed portion of the distal end of the endoscope.

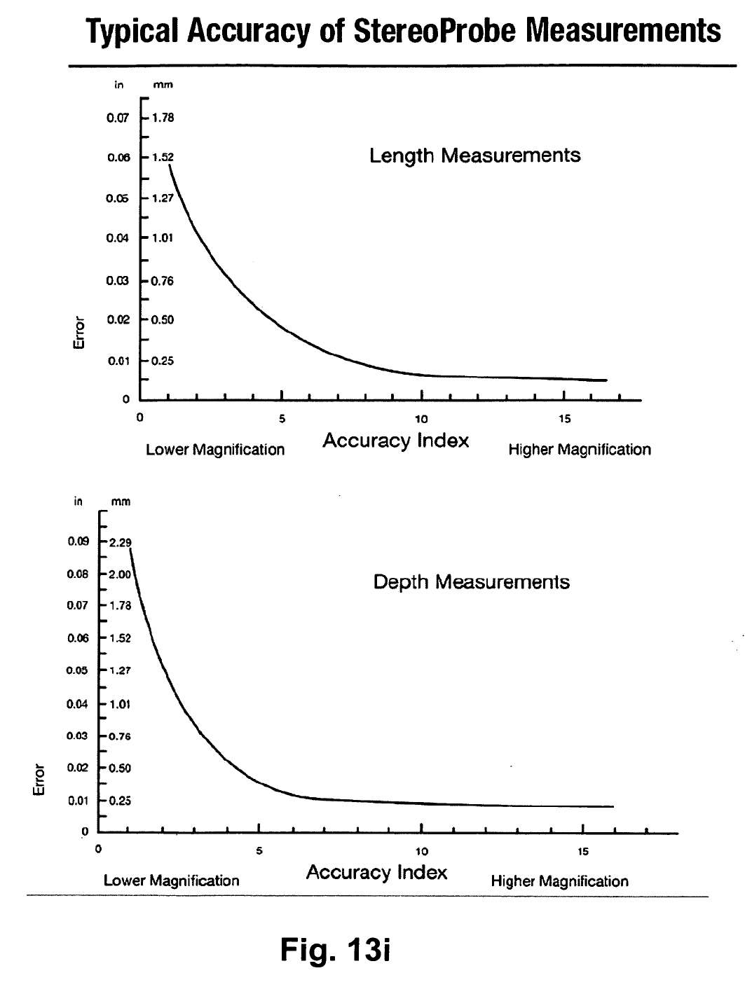

Temperature sensors are used to measure the temperature of the fluid lens and of the white LED. The white LED provides besides illumination a heat source of the fluid lens, if the endoscope is used at temperatures that may influence its optical characteristics. This can be tested and corrected also using digital means and embedded calibration data as shown in the embodiments of FIG. 18.

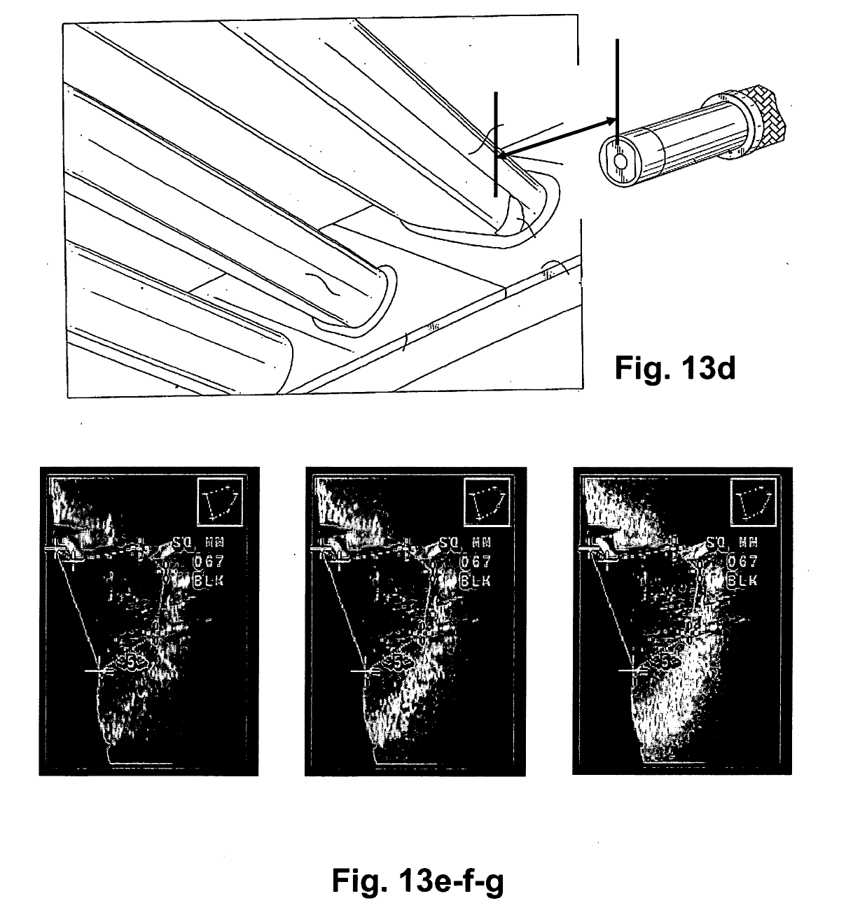

The embodiments of FIG. 16a through FIG. 16c show optical systems for a stereo endoscope using a prism as an image splitter in conjunction with a fluid lens or fluid lenses for auto-focus and autofocus plus optical zooming, or just optical zooming.

In the embodiments of FIG. 16b and FIG. 16c the image is split using fluid lenses.

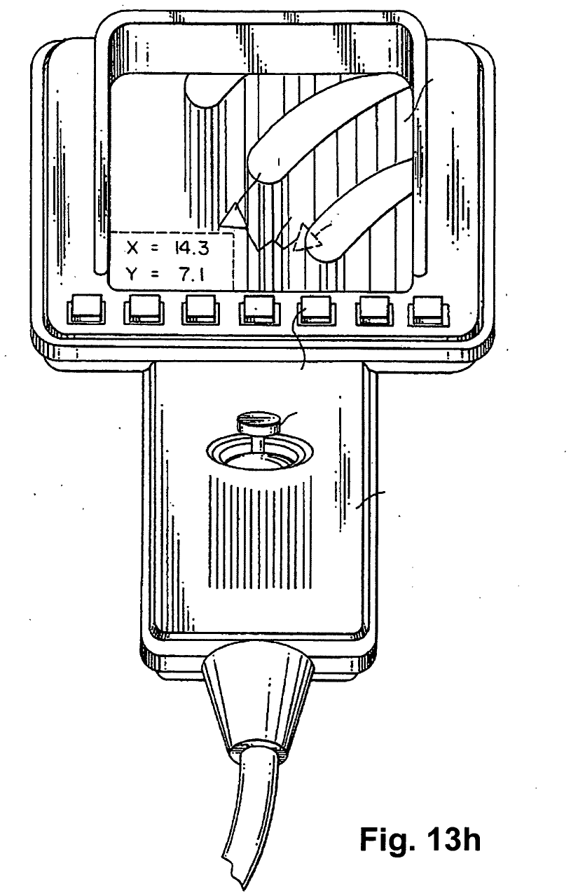

FIG. 17a is a diagram showing a zoom lens configuration that comprises a plurality of fluid lenses.

FIG. 17b is a diagram showing an illustrative ray tracing through a zoom lens system comprising a plurality of fluid lenses.

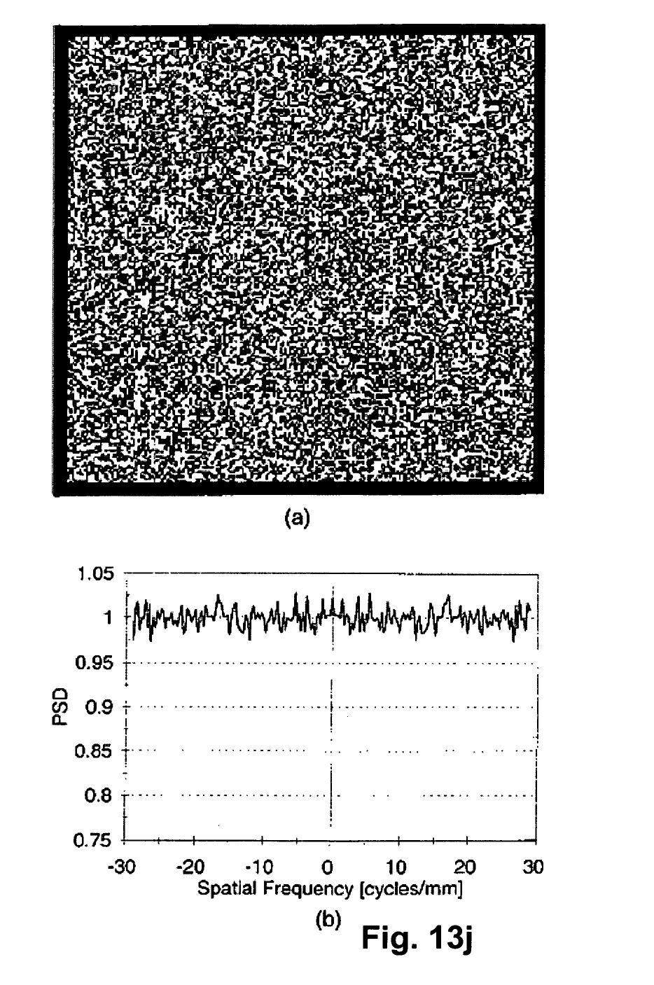

FIGS. 19a and 19b are images of objects being measured using endoscopes according to principles of the invention.

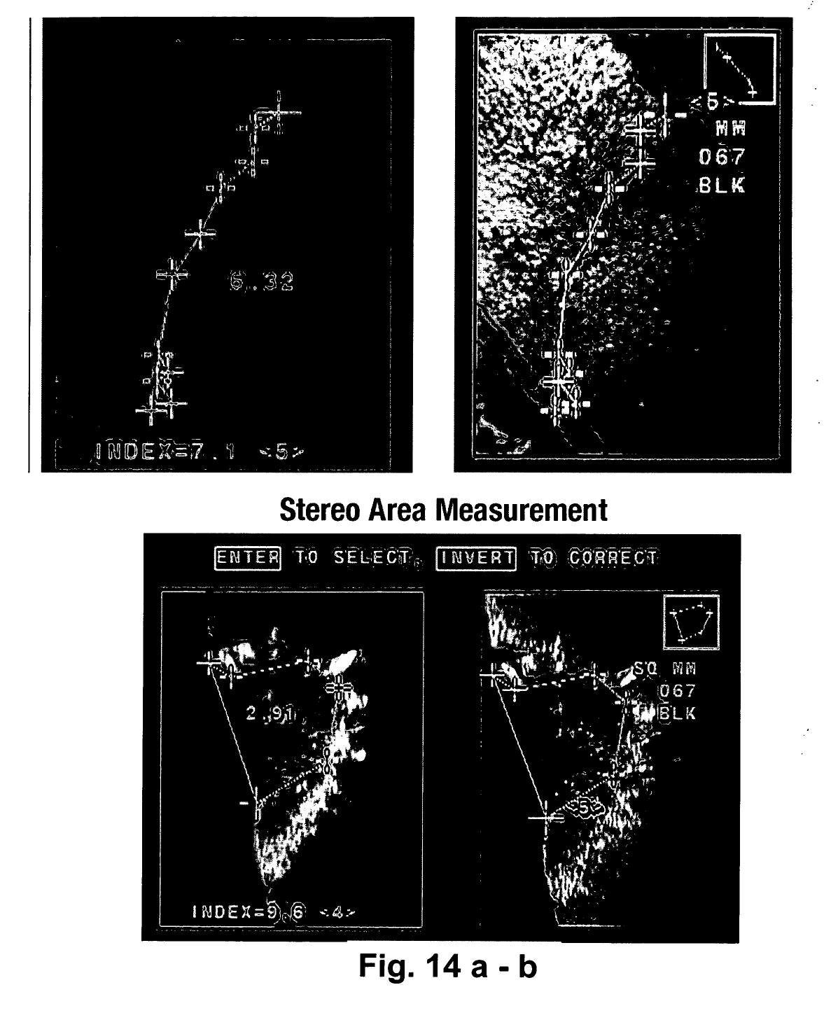

FIG. 20a is a diagram showing in schematic layout the effects of gravitational forces on a fluid lens, and methods of counteracting such forces.

FIG. 20b is a diagram showing the effects of a wedge of glass on the path traversed by a ray of light under different conditions.

FIG. 20c is a diagram showing how a wedge may be provided using three liquids in a fluid lens configuration according to principles of the invention, and how such a wedge affects a ray of light passing therethrough.

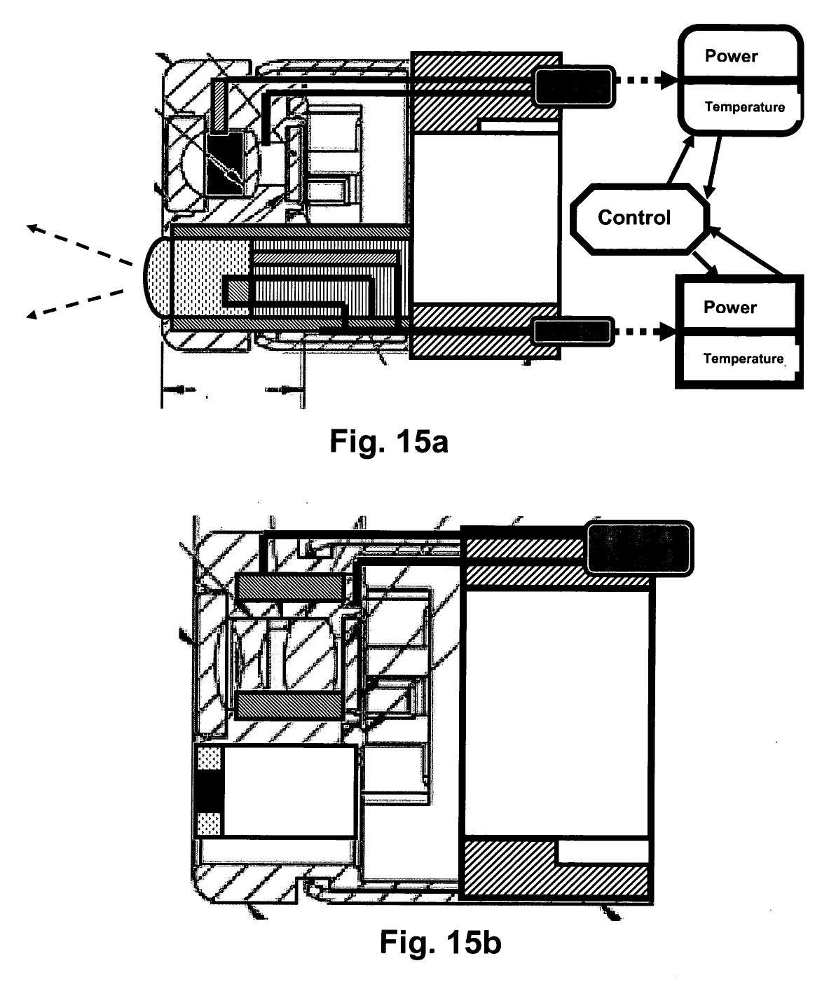

FIG. 21 is a flow chart 1100 showing a process for operating a system having an adjustable focus system comprising feedback. The process begins at step 1110, where a command to capture an image is generated, for example by a user depressing a trigger, or by an automated system issuing a capture image command in response to a specified condition, such as an object being sensed as coming into position for imaging. Once an image is captured at step 1110, the image focus is assessed, as indicated at step 1120. Focus assessment can comprise comparison of the image quality with a specified standard or condition, such as the sharpness of contrast at a perceived edge of a feature in the image, or other standards.

Another procedure for performing an autofocus operation using a flatness metric includes the following steps:

1. capturing a gray scale image (i.e., capture an image with the endoscope and digitize the image using at least two bit resolution, or at least 4 discrete values);

2. optionally sampling the gray scale image (i.e., extract from the image a line or a series of points, or alternatively, the sampled image can be the captured image if it is a windowed frame comprising image data corresponding to selectively addressed pixels);

3. creating a histogram by plotting number of occurrences of data points having a particular gray scale value, for example using the X axis to represent gray scale values and the Y axis to represent frequency of occurrence;

4. processing the histogram to provide a flatness measurement as output;

5. determining a focus level (or quality of focus) based on the flatness measurement; and

6. in the event that the quality of focus as determined from the flatness metric is less than desired, changing the focus and repeating steps 1 through 5.

The flatness of an image refers to the uniformity of the distribution of different gray scale values in the histogram. A flat distribution is one with little variation in numbers of observations at different gray scale values. In general, poorly focused images will be “flatter” than better focused images, i.e. there will be a relatively even incidence of gray scale values over the range of gray scale values. Generally, a histogram for a well focused image has many pixels with high gray scale values, many pixels with low gray scale values, and few pixels in the middle. The use of historical information for various types of images, such as bar codes, including information encoded in look up tables, or information provided using the principles of fuzzy logic, is contemplated

At step 1130, the outcome of the focus assessment is compared to an acceptable criterion, such as sharpness (or contrast change) of a specified amount over a specified number of pixels. Images that are digitized to higher digital resolutions (e.g., using a range defined by a larger number of bits) may support more precise determinations of acceptable focus. If the result of the assessment of focus is negative, the process proceeds to step 1140, where the focus of the lens is modified. After adjusting the focus, the operation of the process returns to step 1110, and a new image is captured, and is assessed. When an image is captured that is found to have suitable focus, the process moves from step 1130 to step 1150, wherein the image with suitable focal properties is processed, and a result is made available to a user or to the instrumentality that commanded the capturing of the image, and/or the result is stored in a memory. Optionally, as indicated at step 1160, the system can be commanded to obtain another image that is to loop back to the step 1110, and to repeat the process again.

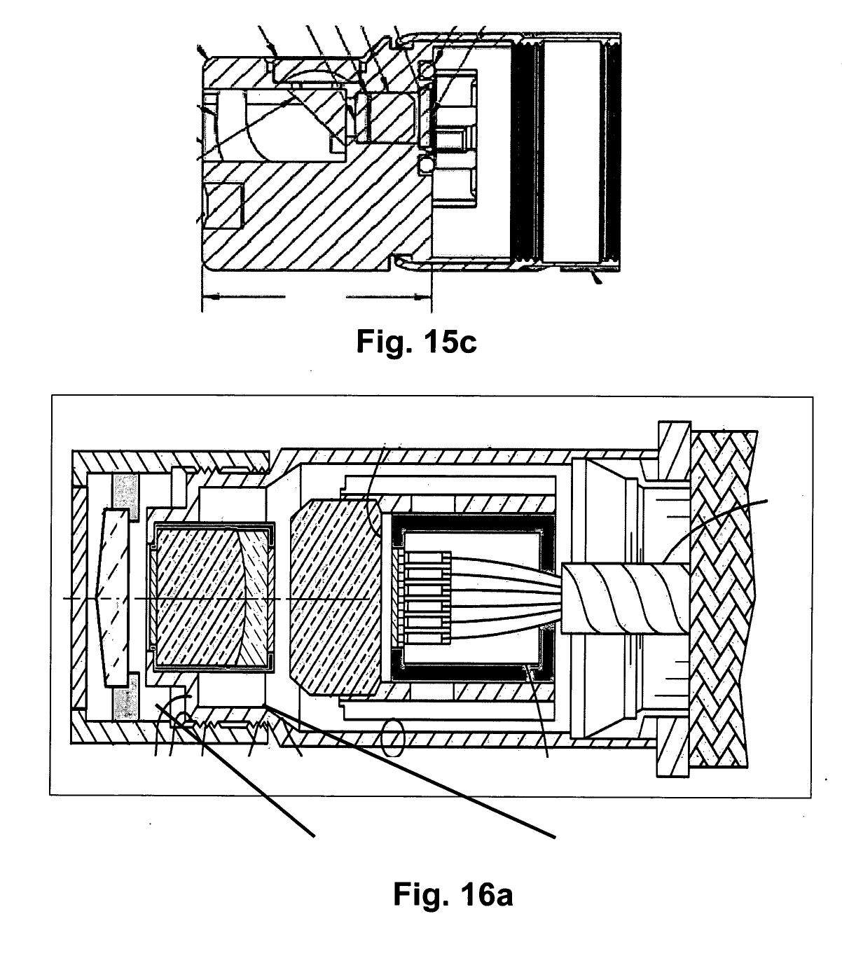

FIG. 22 is a flow chart showing a process for operating a system having an adjustable focus system that does not comprise feedback. At step 1210 a command to capture an image is generated, for example by a user depressing a trigger, or by an automated system issuing a capture image command in response to a specified condition, such as an object being sensed as coming into position for imaging. At step 1215, the lens is driven with a first fluid lens control signal corresponding to a first condition, such as a default condition, for example using a voltage applied to the lens that causes the lens to operate by approximation with focal position q₁ of 10 cm. Using this focal condition, an image is captured and processed at step 1220. At step 1225, the information retrieved from the captured image is examined to determine if a valid image has been achieved. If the decoding is valid, the information or data represented by the image is reported as indicated at step 1260, and the process stops, as indicated at step 1270. A later command to repeat the process can be given as may be necessary or advantageous.

If at step 1225 it is determined that a good image has not been achieved, the process continues to step 1230, at which time the fluid lens control signal applied to the lens is adjusted to a first alternative value, for example a voltage that causes the lens to focus by approximation at a distance q₂ of 30 cm. Using this focal condition, an image is captured and processed at step 1235. At step 1240, the information retrieved from the captured image is examined to determine if a valid image has been achieved. If the image is valid, the information or data represented by the image is reported as indicated at step 1260, and the process stops, as indicated at step 1270.

If at step 1240 it is determined that a good image has not been achieved, the process continues to step 1245, at which time the fluid lens control signal applied to the lens is adjusted to a second alternative value, for example a voltage that causes the lens to focus by approximation at a distance q₃ of 100 cm. Using this focal condition, an image is captured and processed at step 1250. At step 1255, the information retrieved from the captured image is examined to determine if a valid image has been achieved. If the image is valid, the information or data represented by the image is reported as indicated at step 1260, and the process stops, as indicated at step 1270. If a valid image is still not achieved, the process returns to step 1215, and the process is repeated to try to identify a valid image. In other embodiments, after a specified or predetermined number of iterative loops have occurred without a successful outcome, or after a specified or predetermined time elapses, the process can be aborted by a supervisory control device, which in some embodiments can operate according to a computer program. Alternately the process may stop if the trigger is released. Although the process depicted in FIG. 22 uses three discrete conditions to drive the lens in the search for a suitable focus condition, it is possible to use more or fewer than three predefined drive conditions as components of such a process. For example, one can define a process in which the focal distance changes by a predefined distance, or a predefined percentage. Alternatively, one can define a process in which the adjustment is based upon a quantity determined from the information obtained in assessing whether the captured image is in focus (as described hereinabove) or from the quality of the information. In general, the distances specified may not be attained to absolute precision (for example, a distance of 30 cm may not be measured to a precision of 30.000 cm but merely to 30 cm to within one tenth of a centimeter), but rather the test is that the lens operates adequately at the distance that is identified. In the laboratory, precise distances may be set up for experiments, but in actual use in the field, distances are measured less accurately than in the laboratory.

As discussed hereinbefore, fluid lenses may have aberrations, such as spherical aberration and/or color aberration. In an endoscope according to the invention, additional lenses, such as positive or negative lenses, can be used in conjunction with a fluid lens such as lens to correct one or more of spherical, color, or higher order aberrations. In some embodiments, the materials of construction of the additional lenses can be chosen so as to compensate for optical imperfections and aberrations introduced by the fluid lens.

It is expensive to manufacture devices that require high levels of mechanical precision, with regard to making the components of the device, assembling the components with the required precision, and testing the assemble product to assure compliance with the intended design specifications. There are cost and manufacturability advantages that accrue if one is not required to assemble a device with high precision, and can reduce or omit the testing of the assembled device. Accordingly, the incorporation of a fluid lens in the apparatus can in some embodiments permit one or more of relaxed design tolerances, relaxed assembly tolerances, and substitution of a calibration step for a testing step. In some instances, devices that would otherwise have been rejected as being outside of design specifications can be appropriately operated by the simple expedient of operating the fluid lens so as to provide an acceptable level of performance. In particular, one way to assure such a condition is to deliberately design an endoscope in which the baseline operation of the optical system of the apparatus is set for a condition of operation of the fluid lens at an operating point intermediate in the range of operation of the fluid lens. In such an instance, the fluid lens is first driven at the default (or design) condition, and upon calibration, an “adjusted operating condition” different from the default condition can be identified that causes the specific apparatus being calibrated to most closely match the design condition. This “adjusted operating condition” is then recorded as the condition that the apparatus should use as its initial operating state in general operation, and information identifying the “adjusted operating condition” can be saved for future reference, for example in a non-volatile memory. By the application of these design principles (e.g., baseline operation at an intermediate point in the range of operation of the fluid lens), and the associated calibration procedure, individual instruments that might have been rejected as failing a quality assurance test if the design criterion were tighter, and/or if the fluid lens was designed to operate at an extremum of its operating range, can be used satisfactorily by adjusting the base operating condition of the fluid lens in a required direction within the range.

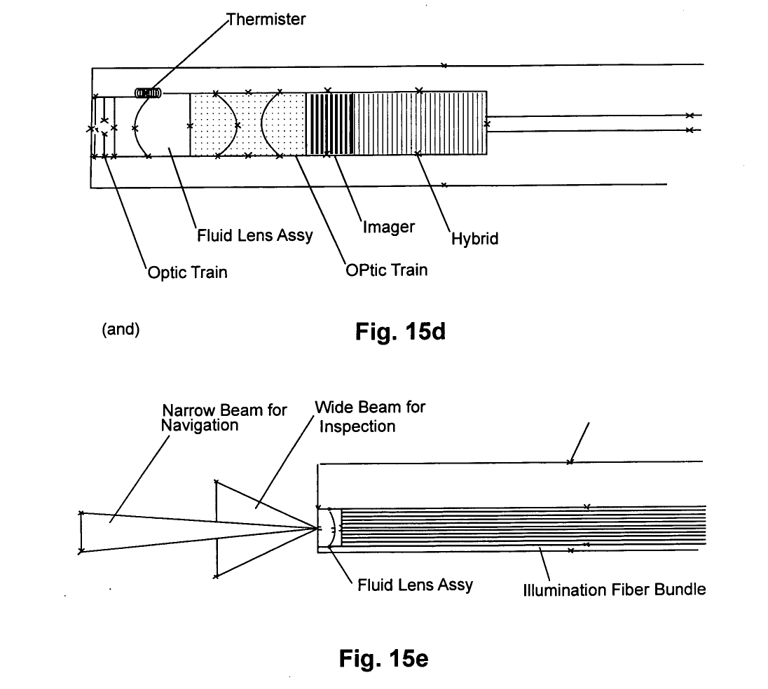

FIG. 23 is a circuit diagram 1300 showing a commutating power supply for a fluid lens system. In FIG. 23, a fluid lens 920 (represented electrically as a capacitor) is connected in a bridge configuration using four switches S1 1310, S2 1312, S3 1314, and S4 1316. The switches in some embodiments are transistors, such as FETs. The bases of the switches S1 1310, S2 1312, S3 1314, and S4 1316 are controlled by a commutator control 1320, so that any of switches S1 1310, S2 1312, S3 1314, and S4 1316 can be set to an open (non-conductive) or closed (conductive) state. A DC power supply 1330 is provided to supply power across terminals 1322 and 1324 of the bridge. A voltage control unit 1332 is provided to control the DC power supply 1330, by providing a control signal, such as a regulated input voltage, to an input terminal of the DC power supply 1330. In some embodiments, a temperature sensor 1334 is provided to sense temperature at the fluid lens 920, at the DC power supply 1330, and/or in the device generally. The temperature sensor 1334 provides a signal to the DC power supply 1330 to adjust the fluid lens control signal applied to the terminals 1322 and 1324 and thereby to the fluid lens 920 to accommodate changes in the operating parameters of the fluid lens 920 as functions of temperature. A computer 1340, which in some embodiments is a microprocessor-based general purpose computer, communicates with all of the commutator controller 1320, the DC power supply 1330, the voltage control unit 1332, and the temperature sensor 1334 by way of a bus 1350. The computer 1340 can be programmed to control all of the components that it communicates with to assure proper operation of the commutating power supply 1300.

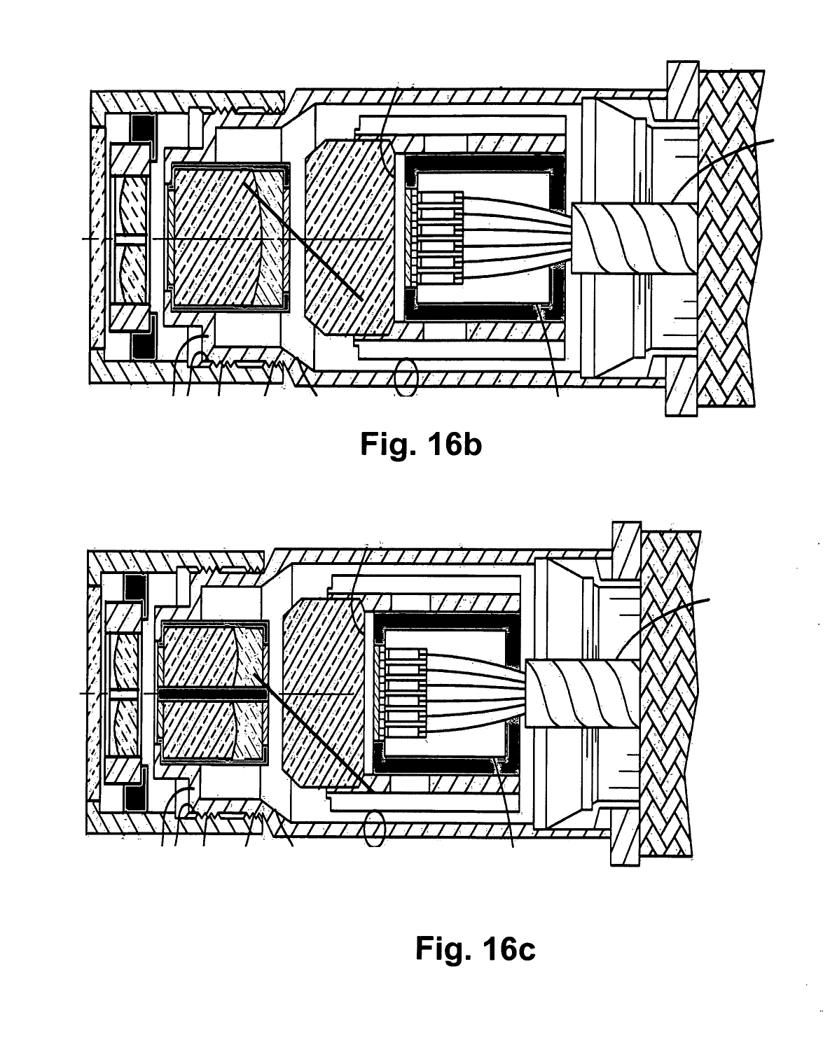

In operation, the commutator controller 1320 provides control signals to the bases of the switches S1 1310, S2 1312, S3 1314, and S4 1316 according to the two states defined in Table I hereinbelow. In state one, switches S1 and S3 are closed, and switches S2 and S4 are open. Accordingly, the positive voltage signal (or positive electric potential) applied to terminal 1322 is conducted to terminal A of the fluid lens 920, and the negative voltage signal (or negative electric potential) applied to terminal 1324 is conducted to terminal B of the fluid lens 920. In state two, switches S1 and S3 are open, and switches S2 and S4 are closed. Accordingly, the positive voltage signal (or positive electric potential) applied to terminal 1322 is conducted to terminal B of the fluid lens 920, and the negative voltage signal (or negative electric potential) applied to terminal 1324 is conducted to terminal A of the fluid lens 920. By periodically switching the signals applied to switches S1 1310, S2 1312, S3 1314, and S4 1316 between states one and two, it is possible to drive the fluid lens 920 with a substantially square wave, as shown in FIG. 24.

TABLE I

| | Switch | Switch | Switch | Switch | | |
|---|---|---|---|---|---|---|
| | S1 | S2 | S3 | S4 | Voltage A | Voltage B |
| State One | Closed | Open | Closed | Open | Positive | Negative |
| State Two | Open | Closed | Open | Closed | Negative | Positive |
| Transition | Open | Open | Open | Open | N.A. | N.A. |

FIG. 24 is a timing diagram 1400 showing a mode of operation of the commutating power supply of FIG. 23. In FIG. 24, the square waves shown can have a repetition period that is variable, and in some embodiments the square waves have a repetition period of approximately 10 milliseconds (ms). As shown in FIG. 24, a period exists between each inversion of the signal applied to the fluid lens 920, which period is termed a transition period or transition interval, and in some embodiments the transition period has a duration of approximately 10 microseconds (μs). In FIG. 24, the time intervals in which voltage A is positive and voltage B is negative correspond to state one, and the time intervals in which voltage A is negative and voltage B is positive correspond to state two. As will be recognized, by the simple expedient of assuring that all switches are open prior to closing any switches, one can avoid applying ill-defined (or undefined) fluid lens control signals to the fluid lens 900. The row of Table I labeled “transition” shows the state of all switches as open, and that the voltages A and B are N.A., which represents “none applied.” In addition, the duration of any state can be controlled to be any duration between the switching time of a switch (that is, the time it takes the switch to switch states) at the short duration limit to the time one elects to apply a particular state at the long duration limit. Also, there is no requirement that states one and two have the same duration, although that is one possibility.

By controlling the behavior of the fluid lens in the apparatus, it is possible to calibrate the operation of the fluid lens by recording the observed control signal (such as a voltage or impressed electric potential) that is required to obtain an acceptable (e.g., an image within an acceptable range of image quality), and preferably optimal, image of the target at each location or position.

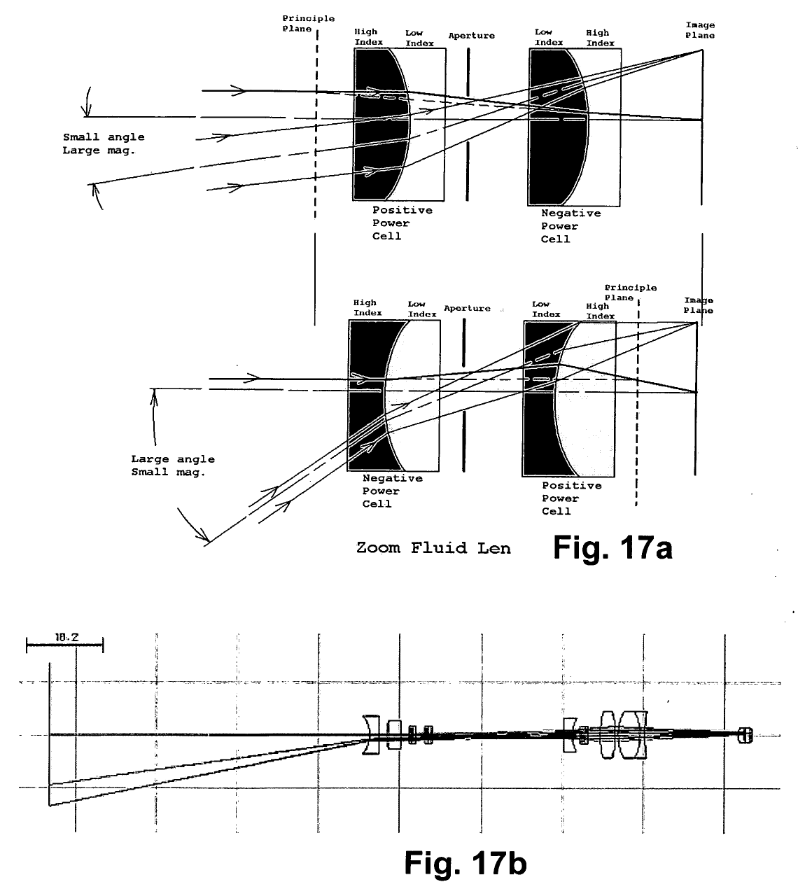

FIG. 25 is a flow chart 1700 of a calibration process useful for calibrating an apparatus. In FIG. 25, the calibration is initiated, as shown at step 1705, by initializing the system, including performing all power-on-sequence tests to assure that the system components are operating properly. At step 1710, a test target bearing a pattern or encoded symbol is positioned at a first test position. When in the first test position, the target will in general be at defined distance and orientation relative to the endoscope comprising a fluid lens. At step 1715, the fluid lens control signal (which in some embodiments is a voltage) is adjusted to obtain an acceptable, and preferably an optimal, focus condition for the target. At step 1720, the distance and orientation of the target and the fluid lens control signal parameters (for example magnitudes and signs of voltages, timing features of the signal such as pulse duration, transition time and repetition rate) are recorded for future use in a non-volatile memory, for example in a table.

One can iteratively repeat the process steps of locating the target at a new location and orientation, controlling the fluid lens control signal applied to the fluid lens to obtain a satisfactory, and preferably optimal, focus, and recording in a memory the information about the target location and orientation and the fluid lens control signal parameters, so as to provide a more complete and detailed set of calibration parameters. The number of iterations is limited only by the amount of time and effort one wishes to expend performing calibration steps, and the amount of memory available for recording the calibration parameters observed. A calibration according to the flow diagram of FIG. 25 would include performing calibration steps as described by steps 1710, 1715 and 1720 at three distinct positions for the target. The information obtained in calibration tests can be used when operating the corresponding imager (or in some instances, another imager of similar type) either by using the calibration information as an initial setting for operation in a closed loop mode as explained in connection with FIG. 21, or as fixed operating conditions for discrete points in an open loop operating mode as explained in connection with FIG. 22.

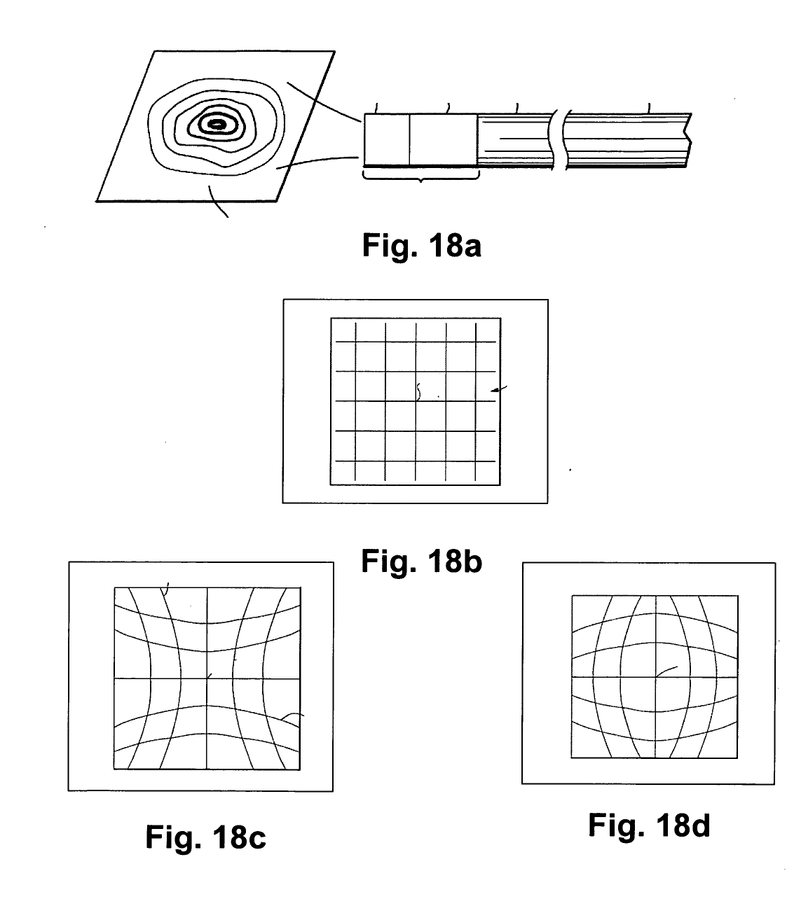

FIG. 26 is a diagram 1800 showing calibration curves for a plurality of exemplary endoscopes. In FIG. 26, the horizontal axis 1802 represents a fluid lens control signal parameter, such as voltage, and the vertical axis 1804 represents an optical property of the fluid lens, such as optical power. One can also represent other optical properties of a fluid lens that are relevant for its operation, such as focal length, f-number, and deviation from a default optical axis (which default optical axis may be considered to represent zero degrees of elevation or altitude and zero degrees of azimuth). In FIG. 26, three curves 1810, 1812, 1814 are shown, each curve representing a response (e.g., optical power) of a specific fluid lens to an applied fluid lens control signal (e.g., voltage). As seen in FIG. 26, the curve 1810, representing the behavior of a first fluid lens, reaches an optical power P 1820 at an applied voltage V₁ 1830. However, other fluid lenses may behave slightly differently, such that a second fluid lens, represented by curve 1812, attains optical power P at an somewhat larger voltage V₂ 1832, and a third fluid lens, represented by curve 1814, attains optical power P at yet a larger voltage V₃ 1834. Accordingly, one can extract from the information in FIG. 26 a relation between the fluid lens control signal that is to be applied to the first fluid lens and the second fluid lens to attain the same optical power P, for example for operating two endoscopes under substantially similar conditions, or for operating a binocular endoscope or other device that uses two fluid lenses simultaneously, for example to generate a stereoscopic view of a target. At power P, there exists a difference in drive voltage between the first lens and the second lens given by V₂−V₁, where the difference has a magnitude given by the absolute value of V₂−V₁ and a sign which is positive if V₂ exceeds V₁ in magnitude, negative if V₁ exceeds V₂ in magnitude, and zero if V₂=V₁. In operation, in order to attain optical power P in both of the first and second fluid lenses, one can provide a fluid lens control signal equal to V₁ to both the first and second fluid lenses, and a differential signal equal to the signed difference of V₂−V₁ to the second fluid lens. Alternatively, one could use two power supplies that provide signals V₁ and V₂ to the first and second fluid lenses, respectively. As the optical power required for operation of a fluid lens changes, the fluid lens control signal changes, and can be deduced or read from the appropriate curve of FIG. 26. Since one in general does not measure the parameters of a fluid lens or other device at all possible values within a range, a curve such as 1810 can also be obtained by measuring a discrete number of pairs of optical parameter and associated fluid lens control signal, and fitting a curve to the data, or interpolating values between adjacent data points, as may be most convenient to prepare a suitable calibration curve. In some instances, only a single calibration point per fluid lens module may be required. Rather than creating curves for different fluid lenses, one can measure the same fluid lens at different temperatures. Then the appropriate operating point can be determined at the various temperatures. Other operating points may be determined by either extrapolation or interpolation, by suitable curve fitting relationships, or by deducing a representation of the behavior in the form of an equation.

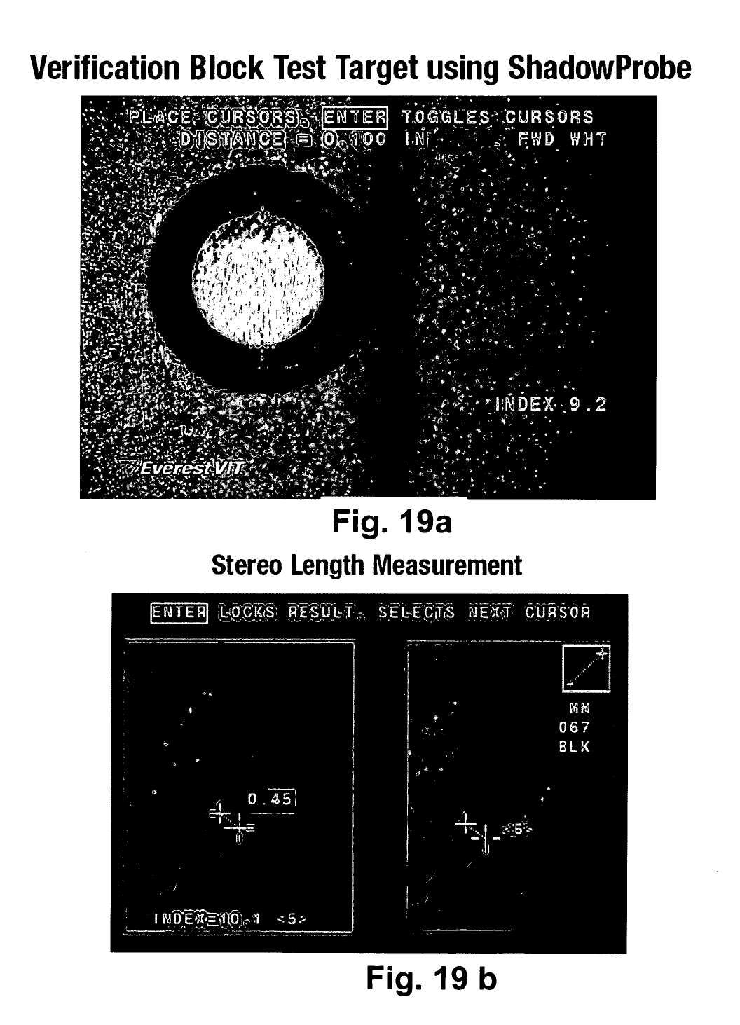

FIG. 27 is a diagram showing an embodiment of a power supply 1900 suitable for use with endoscopes. In general, the first order electrical equivalent circuit for a fluid lens is a simple capacitor. In FIG. 27, a load 1910 represents in one embodiment a capacitive load to a power supply, generally 1920. Because the load is capacitive, the net power consumed is in general small. The power supply 1920 of FIG. 27 is one possible embodiment, which is described first at a high level. The output of this power supply can be used as input to the commutator shown in FIG. 23 comprising switches 1310, 1312, 1314, and 1316. A power source, such as a 6 volt battery 1922, is adequate for operation of the supply. The voltage of the power source may be increased using a DC-to-DC converter comprising a switcher IC 1930 having a sensing terminal, a controller for a switch 1940, (such as a transistor) and an inductor 1935 (which may be provided externally to the switcher). The sense terminal in some embodiments is connected to a voltage divider 1955. A rectifier 1945 is used to provide a unipolar output, which includes noise introduced by the switching operation of the switcher. The output voltage of the first stage of the power supply can be controlled, and in general will be of the order of tens of volts, for example 60V DC. A filter 1960, such as a low pass RC filter, is provided to eliminate noise, as the capacitive elements represent a small impedance as frequency is increased, and represent a large (substantially infinite) impedance to low frequencies. A precision low noise series regulator 1970 is used to control the output voltage for example by controlling a transistor 1972, with a sense input to the series regulator providing a feedback loop through voltage divider 1975. A control 1984 is provided to permit adjustment of the voltage signal applied to the fluid lens, and thereby providing control of a focal distance or plane of focus of the fluid lens 1910. Alternative power supplies that can provide a unipolar output can be used. By using a pair of power supplies (e.g., one providing a positive voltage and one providing a negative voltage), a single power supply and a suitably biased inverter, or by using a single power supply and dual operational amplifiers, one can provide a pair of outputs that are symmetric relative to ground.

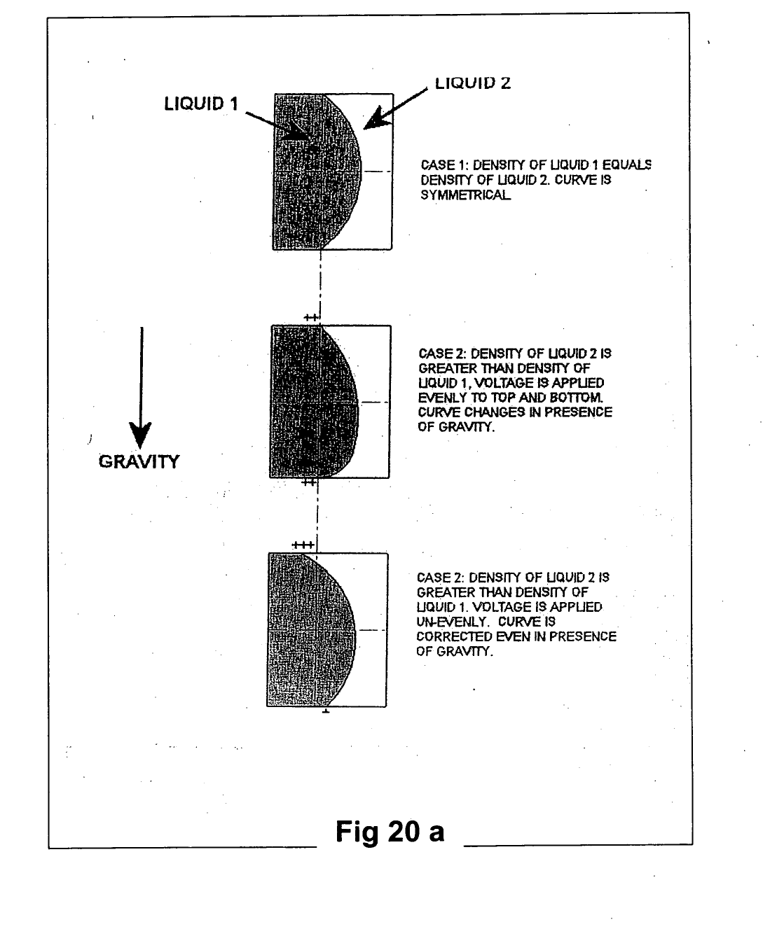

FIG. 28 is a timing diagram 2000 illustrating an exemplary mode of operation of an endoscope comprising a fluid lens. Three types of signals are shown in FIG. 28. One compound signal 2010, 2020 is similar to that already described with respect to FIG. 24 hereinabove. The components 2010, 2020 are square waves applied to the terminals of a fluid lens using a commutating connection as described in FIG. 23, in which the power supply is a unipolar power supply of FIG. 27. In FIG. 28, a driving voltage of magnitude V₁ produces a first focus location for the fluid lens, while a driving voltage of magnitude V₂ produces a second focus location for the fluid lens. An illumination signal 2030 is shown, which indicates the timing of a control signal applied to one or more illumination sources such as LEDs present in the endoscope, for illuminating a target or object of interest. The illumination signal 2030 is shown as a series of square pulses, whereby the LEDs are turned on to provide illumination for a portion of a inspection cycle, rather than having the LEDs operating at all times, which wastes power. A signal 2040 is shown that represents the integration period for the image sensor array. Signal 2040 is also a series of pulses. The pulses that operate the image sensor array begin after the illumination signal 2030 is switched “on,” and are switched “off” at least as early as the illumination signal pulses are turned off. By preventing the image sensor from operating during the delay time T_{d}, one minimizes or eliminates the likelihood of introducing optical error, or “blur” caused by a changing focus of the fluid lens while the image sensor is operating. There is illumination provided during an interval when the image sensor is operative to capture the illumination from the object, which illumination is in at least some embodiments provided by the illumination source. In order to operate at 30 frames per second (the typical video frame rate in the U.S.), the lens drive voltage signal must operate on a cycle of not longer than 33.3 ms per repetition, as shown in FIG. 28. It is advantageous to provide a brief delay period T_{d} in order to provide a decay time for any transients in the fluid lens that may be induced by a change in applied fluid lens control signal (e.g., to allow transients to wash out prior to using the fluid lens after a change in fluid lens control signal has been applied). The LEDs or other illumination sources can be activated during the delay time T_{d} so as to have the illumination available when the image sensor is made operational. In many embodiments, the image sensor operates in a brief enough time period that it does not have to be operated during the later portion of a 33.3 ms interval. The time scale of the illumination pulses and of the image sensor activation can in some embodiments be as short as 1 ms advantageously, but even shorter times are possible.

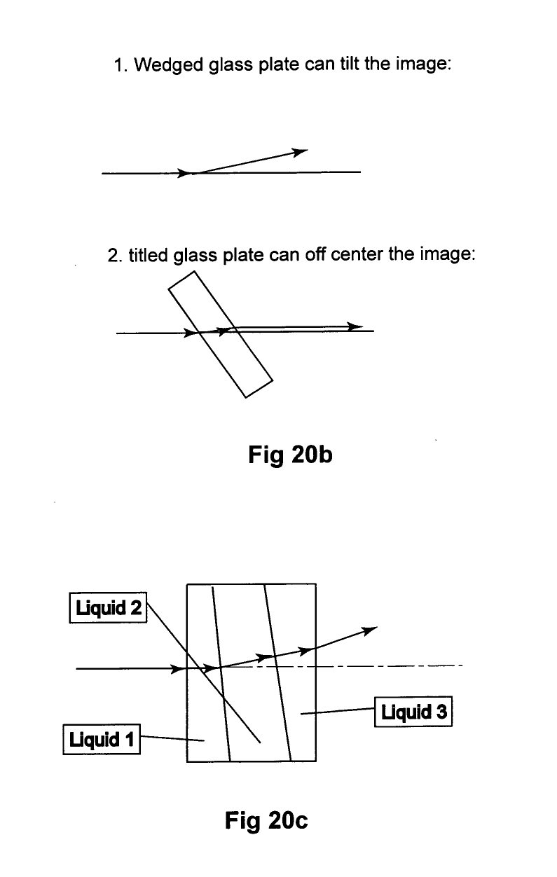

FIG. 29a through FIG. 29c are cross-sectional drawings showing an exemplary fluid lens 2100 with a mount comprising an elastomer for an endoscope. Such elastomers are made by Chomerics North America, Parker Hannifin Corp., 77 Dragon Court, Woburn, Mass. 01801. In FIG. 29a, a fluid lens 2110 is shown with a solid body 2112 in the form of a ring, and electrical contacts 2114, 2116 disposed on opposite sides thereof. In some embodiments, the fluid lens body 2112 is made of metal, and can also represent one of the contacts 2114, 2116, the other contact being insulated from the metal body 2112. In other embodiments, the body 2112 is made from, or comprises, a non-conducting substance.

In FIG. 29b, the fluid lens body 2112 is shown mounted in a holder 2120. In one embodiment, the holder 2120 is tubular and has an internally threaded surface 2130 and a partially closed end 2132 having defined therein an aperture of sufficient size not to occlude the optically active portion of the fluid lens. The fluid lens body 2112 is held in place by a threaded retainer ring 2122 that threadedly mates with the internally threaded surface 2130 of the holder 2120. The holder 2120 and retainer ring 2122 are made of an insulating material. In some embodiments, an elastomeric material 2140, 2142 is provided in the form of an “O” ring or an annular washer, so that the fluid lens is supported in a desired orientation, without being subjected to excessive compressive forces or to mechanical disturbances that can be accommodated by the elastomeric ring 2140, 2142. In some embodiments, a single elastomeric ring 2140 or 2142 is provided on one side of the fluid lens body 2120. In some embodiments, one elastomeric ring 2140 is provided on one side of the fluid lens body 2120, and a second elastomeric ring 2142 is provided on the other side of the fluid lens body. Electrical contact with the contacts 2114 and 2116 is provided by wires 2114′ and 2116′ that contact the respective contacts and which exit the holder. These wires are in intimate electrical contact with the elastomeric material 2122 and 2140. As needed, wires 2114′ and 2116′ can be insulated. FIG. 29c shows the elastomeric washer 2140, which in some embodiments can be conductive, in contact with a fluid lens body 2112 at an electrical contact 2116 thereof, which fluid lens body 2112 is supported in a holder 2120 at a partially closed end 2132 thereof. A wire 2116′ contacts the conductive elastomeric washer or ring 2140 and exits the holder 2120 by way of an aperture 2134 defined within the holder 2120. In some embodiments, the wire 2116′ contacts the electrical contact of the fluid lens body, and the elastomeric ring or washer is positioned between the wire 2116′ and the partially closed end 2132 of the holder 2120. In other embodiments, the wire 2116′ is between the elastomer 2140 and the partially closed end 2130. The holder 2120 and threaded ring 2122 can be constructed of any suitable material, and can be non-conductive or conductive as appropriate.

The present invention also deals with the deleterious effects of image smear caused by hand jittering or hand motion in a hand held imager. Image smear has been one of the major sources for image quality degradation. Image smear and similar degradation mechanisms cause a reduced decode rate in a barcode reading application or a reduced contrast and a blurry image in an image capturing application. In some instances, hand jitter or hand motion can cause image degradation that may be severe enough to prevent the image from being processed correctly.

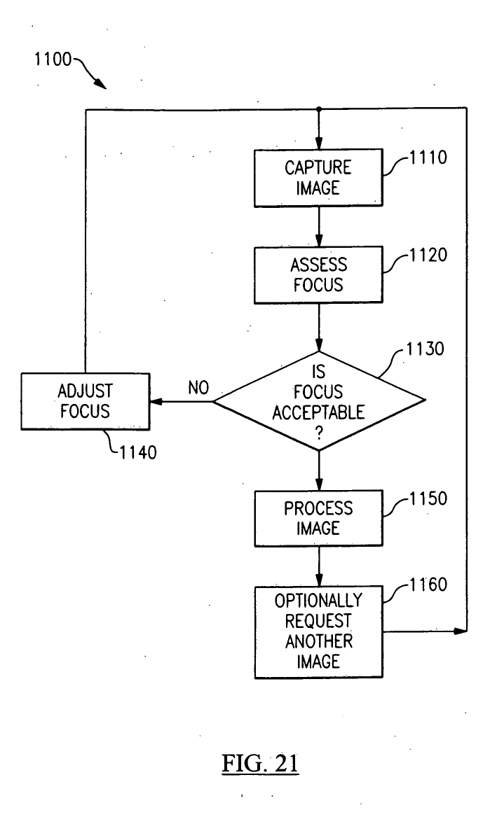

FIG. 30 is a diagram illustrating a prior art variable angle prism as disclosed in U.S. Pat. No. 6,734,903 to Takeda, et. al. (hereinafter “the '903 patent”). The apparatus disclosed employs two angular velocity sensors, two angular sensors, two actuators and a variable angle prism with a lens system to form an anti-shaking optical system. This type of optical system is widely used in hand held video camcorders to correct the hand jittering effect. However, such systems suffer from a variety of drawbacks, including: 1. higher cost due to many parts; 2. slow response time due to the use of mechanical actuators; 3. lower reliability due to moving parts; 4. the use of a separate auto-focusing electromechanical subsystem that further increases the cost and system complexity; and 5. the use of mechanical components that increases the complexity and difficulty of assembly.

The '903 patent describes the operation of the variable angle prism as is expressed in the following 11 paragraphs.

A camera shake is a phenomenon in which photographed images move vertically or horizontally while a user is performing photographing by holding a video camera in his or her hands, since the hands or the body of the user slightly moves independently of the user's intention. Images thus photographed can give a viewer considerable discomfort when reproduced on a television monitor or the like.

To avoid this camera shake phenomenon, conventional video cameras make use of, e.g., a variable angle prism (to be referred to as a “VAP” hereinafter).

A practical example of an arrangement of a conventional image sensing apparatus including a VAP for camera shake correction will be described below with reference to FIG. 30.

In FIG. 30, a VAP 2204 is constituted by coupling two glass plates 2204a and 2204b via a bellows-like spring member 2204c and sealing an optically transparent liquid 2204d in the space surrounded by the two glass plates 2204a and 2204b and the spring member 2204c. Shafts 2204e and 2204f provided in the glass plates 2204a and 2204b are connected to an actuator 2203 for horizontal driving and an actuator 2208 for vertical driving, respectively. Therefore, the glass plate 2204a is rotated horizontally, and the glass plate 2204b is rotated vertically.

Note that the VAP 2204 is described in Japanese Patent Laid-Open No. 2-12518 and so a detailed description thereof will be omitted.

A horizontal angular velocity sensor 2201 detects an angular velocity caused by a horizontal motion of the image sensing apparatus resulting from a camera shake or the like. A control unit 2202 performs an arithmetic operation for the detection signal from the angular velocity sensor 2201 such that this horizontal motion of the image sensing apparatus is corrected, and detects and supplies an acceleration component to the actuator 2203. This actuator 2203 drives the glass plate 2204a of the VAP 2204 horizontally.

The rotational angle of the glass plate 2204a which can be horizontally rotated by the actuator 2203 is detected by an angle sensor 2205. The control unit 2202 performs an arithmetic operation for this detected rotational angle and supplies the result to the actuator 2203.

A vertical angular velocity sensor 2206 detects an angular velocity caused by a vertical motion of the image sensing apparatus resulting from a camera shake or the like. A control unit 2207 performs an arithmetic operation for the detection signal from the angular velocity sensor 2206 such that this vertical motion of the image sensing apparatus is corrected, and detects and supplies an acceleration component to the actuator 2208. This actuator 2208 drives the glass plate 2204b of the VAP 2204 vertically.

The rotational angle of the glass plate 2204b which can be vertically rotated by the actuator 2208 is detected by an angle sensor 2209. The control unit 2207 performs an arithmetic operation for this detected rotational angle and supplies the result to the actuator 2208.

An image sensing optical system 2210 forms an image of an object to be photographed on an image sensor 2211. This image sensor 2211 is constituted by, e.g., a CCD. A two dimensional solid state CCD is used in conventional image sensing apparatuses such as video cameras. An output from the image sensor 2211 is output to a recording apparatus or a television monitor through a signal processing circuit (not shown).

In the conventional image sensing apparatus with the above arrangement, the horizontal and vertical angular velocities caused by a camera shake are detected. On the basis of the angular velocities detected, the actuators move the VAP horizontally and vertically to refract incident light, thereby performing control such that the image of an object to be photographed does not move on the image sensing plane of the image sensor. Consequently, the camera shake is corrected.

In the current invention, a fluid lens provided with additional components to counteract involuntary motions (“an anti-hand-jittering fluid lens”) combines the auto-focusing and variable angle prism functionality into a single low cost component that has no moving parts, and that provides fast response time.

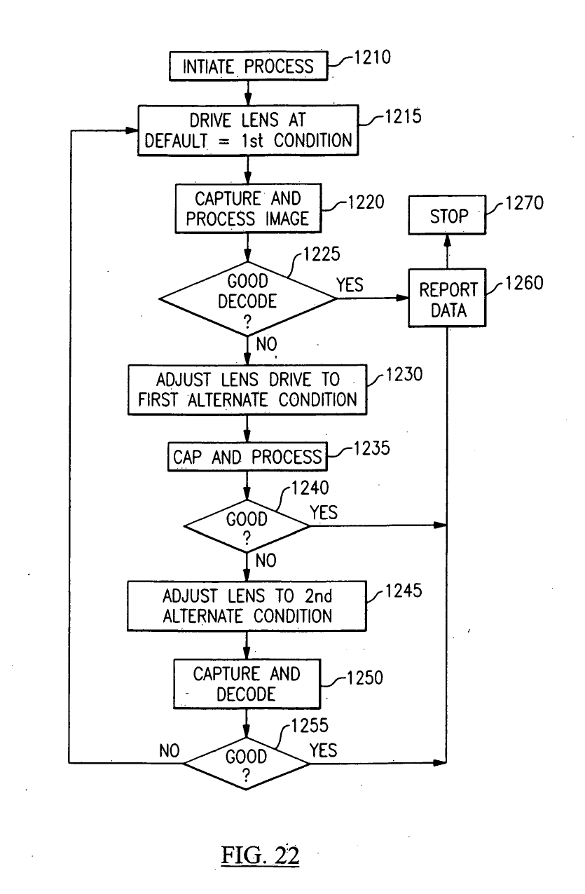

FIG. 31 is a cross-sectional diagram 2300 of a prior art fluid lens that is described as operating using an electrowetting phenomenon. The fluid lens 2300 is a substantially circular structure. The fluid lens comprises transparent windows 2302, 2304 on opposite sides thereof. In FIG. 31, a drop of conductive fluid 2360 (such as water), possibly including dissolved electrolytes to increase conductivity, or to adjust the density of the conductive fluid to match the density of another fluid 2370 that is immiscible with the conductive fluid (such as oil), is deposited on a surface, such as a window. A ring 2310 made of metal, covered by a thin insulating layer 2312 is adjacent the water drop. A voltage difference is applied between an electrode 2320 (that can also be a ring) and the insulated electrode 2310, as illustrated by the battery 2330. In some embodiments, an insulating spacer 2335 (not shown) is located between the rings 2310 and 2320. The voltage difference modifies the contact angle of the liquid drop. The fluid lens uses two isodensity immiscible fluids; one is an insulator (for example oil) while the other is a conductor (for example water, possibly with a salt dissolved therein), which fluids touch each other at an interface 2340. The variation of voltage leads to a change of curvature of the fluid-fluid interface 2340, which in turn leads to a change of the focal length or power of the lens as a result of the refraction of light as it passes from one medium having a first optical index to a second medium having a second, different, optical index. In the embodiment shown, an optical axis 2350 is indicated by a dotted line lying substantially along an axis of rotation of the fluid lens 2300. Although the power of the fluid lens, or its focal length, can change by application of suitable signals to the rings 2310 and 2320, which signals cause the curvature of the interface 2340, in the embodiment shown in FIG. 31 there is no convenient way to cause the optical axis to deviate away from the axis of rotation of the fluid lens in a deliberate manner or by a desired angle.

The current invention uses the principle of altering the interface shape between two fluids and provides another voltage (or other suitable fluid lens control signal) to control an optical tilt of the fluid interface to adjust an exit optical axis angle or direction relative to the fluid lens. One application of such adjustment of the exit optical axis angle is to provide a mechanism and method to compensate the angular movement caused by hand-jittering or hand motion.

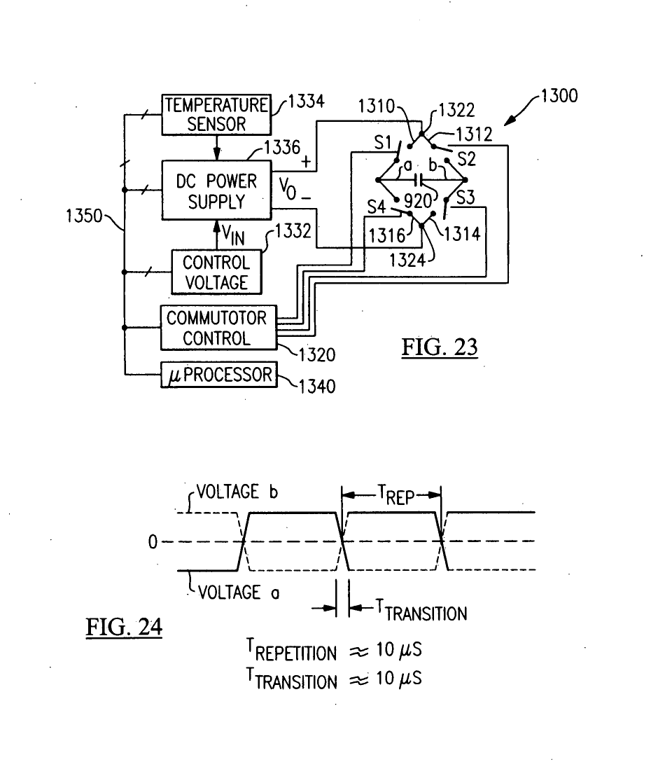

FIG. 32a is a cross sectional diagram 2400 showing an embodiment of a fluid lens configured to allow adjustment of an optical axis, and FIG. 32b is a plan schematic view of the same fluid lens. FIG. 32b indicates that the two metal ring electrodes 2310, 2320 of the prior art fluid lens shown in FIG. 31 have been divided into a plurality of segments, for example four arc pairs (2410a, 2420a), (2410b, 2420b), (2410c, 2420c) and (2410d, 2420d). A plurality of controllable signal sources, such as voltage sources V1, V2, V3, and V4, are provided, such that each controllable signal source can impress a signal on a selected pair of electrodes independent of the signal applied to any other electrode pair. In order to generate a desired curvature of the fluid interface 2440 in the fluid lens 2400, one can control all four voltage controls V1, V2, V3, and V4 to apply a uniform focusing voltage Vf. In this mode of operation, the fluid lens 2400 functions in exactly the same manner as the prior art fluid lens shown in FIG. 31. However, to generate an optical tilt (or to adjust an optical axis of the fluid lens 2400) using the fluid lens of the current invention, in one embodiment, a horizontal tilt voltage dh and a vertical tilt voltage dv are applied on each of the voltage controls by superimposing the tilt voltages on top of the focusing voltage Vf according to the following equations:
V1=Vf+dv
V2=Vf+dh
V3=Vf+dv
V4=Vf+dh
Application of these new signals V1, V2, V3 and V4 creates a two-dimensional tilted fluid lens, in which horizontal and vertical tilt angles are determined according to the magnitudes and signs of the control voltages dh and dv. One can generate such signals involving superposition of a signal Vf and an adjusting signal using well known circuits that are referred to as “summing circuits” in analog circuit design, and by using a digital controller such as a microprocessor-based controller and a digital-to-analog converter to generate suitable fluid lens control signals using digital design principles. In FIG. 32A, fluid lens surface 2445 is shown with a tilt in the vertical dimension caused by application of a signal dv as indicated for V1 and V3. The optical axis 2450 of the undeviated fluid lens is shown substantially along the axis of rotation of the fluid lens, and the deviated or adjusted optical axis is shown by dotted line 2455, which is asymmetric with regard to the axis of rotation. Notice that surface 2445 not only provides focusing curvature to provide a desired optical power of focal length, but also pervades a mechanism to adjust the optical axis to correct for the hand jittering or hand motion. In other embodiments, other applications can be contemplated. As an example, one can set the focal length of the lens to a small value (e.g., operate the lens as a “fisheye” lens that has a wide field of view and great depth of field) and use the adjustment of the optical axis to tip the field of view to bring some feature of interest within the field of view closer to the center of the field of view. In a fisheye lens, features in the center of the field as observed with minimized optical distortions relative to the edge of the field of view, so the object of interest can be observed with reduced distortion. Additionally, a fisheye lens typically spreads out objects at the edge of the field of view, so such operation can increase the number of pixels that the object of interest occupies on a planar image sensor, thereby increasing the detail that may be resolved.

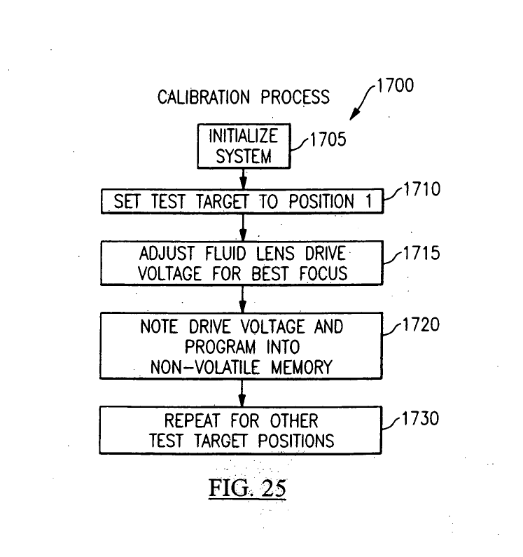

FIG. 33 is a schematic diagram 2500 showing the relationships between a fluid lens and various components that allow adjustment of the optical axis direction. The optical axis control system comprises a horizontal angular velocity sensor 2510, a control module 2512 to generate horizontal tilt voltage dh, a vertical angular velocity sensor 1520, a control module 2522 to generate vertical tilt voltage dv, an auto-focusing control module 2530 to generate a focusing voltage Vf, a distributor module 2540 to synthesize the control voltages to control the fluid lens module 2400 to accommodate or to correct for hand jittering. Alternately when the axis of the optical system changes orientation, the image on the image sensor will move. The processor can extract the magnitude and direction of motion of the object that was not expected to move. This can be used as input to the correction circuit.

In some embodiments, the angular velocity sensors 2510 and 2520 are commercially available low cost solid-state gyro-on-a-chip products, such as GyroChips manufactured by BEI Technologies, Inc., One Post Street, Suite 2500 San Francisco, Calif. 94104. The GyroChip comprises a one piece, quartz micromachined inertial sensing element to measure angular rotational velocity. U.S. Pat. No. 5,396,144 describes a rotation rate sensor comprising a double ended tuning fork made from a piezoelectric material such as quartz. These sensors produce a signal output proportional to the rate of rotation sensed. The quartz inertial sensors are micromachined using photolithographic processes, and are at the forefront of MEMS (Micro Electro-Mechanical Systems) technology. These processes are similar to those used to produce millions of digital quartz wristwatches each year. The use of piezoelectric quartz material simplifies the sensing element, resulting in exceptional stability over temperature and time, and increased reliability and durability.

In other embodiments, it is possible to divide the two metal rings 2410 and 2420 of FIG. 32B into more than four symmetric arc pairs to create more smooth tilt fluid lens. For example, one of the embodiments can have 12 symmetric arc pairs layout in a clock numeric topology. All the system components shown in FIG. 33 will be the same except that the output of distributor 2540 will have 12 voltage control outputs to drive the 12 arc pairs of the fluid lens module. The voltage synthesis algorithm in distributor 2540 is based on the gradient of a (dh, dv) vector. For example, viewing the fluid lens as if it were a clock, (dh, dv)=(2.5, 0) will have a highest voltage output at a pair of electrodes situated at the 3-o'clock position and the lowest voltage output at a pair of electrodes situated at the 9-o'clock position, and no superimposed voltage would be applied to the electrode pairs nearest the 12-o'clock and 6-o'clock positions. It is possible to interpolate the gradient across any intermediate pairs of electrodes around the circle so as to apply a smoothly varying fluid lens control signal. In principle, one could build a fluid lens with as many electrode pairs as may conveniently be provided. In some embodiments, one of the two ring electrodes can be a continuous ring to provide a common reference voltage for all of the pairs, one element of each pair being the continuous ring, which for example might be held at substantially ground potential, for ease of mounting and assembly, if for no other reason.

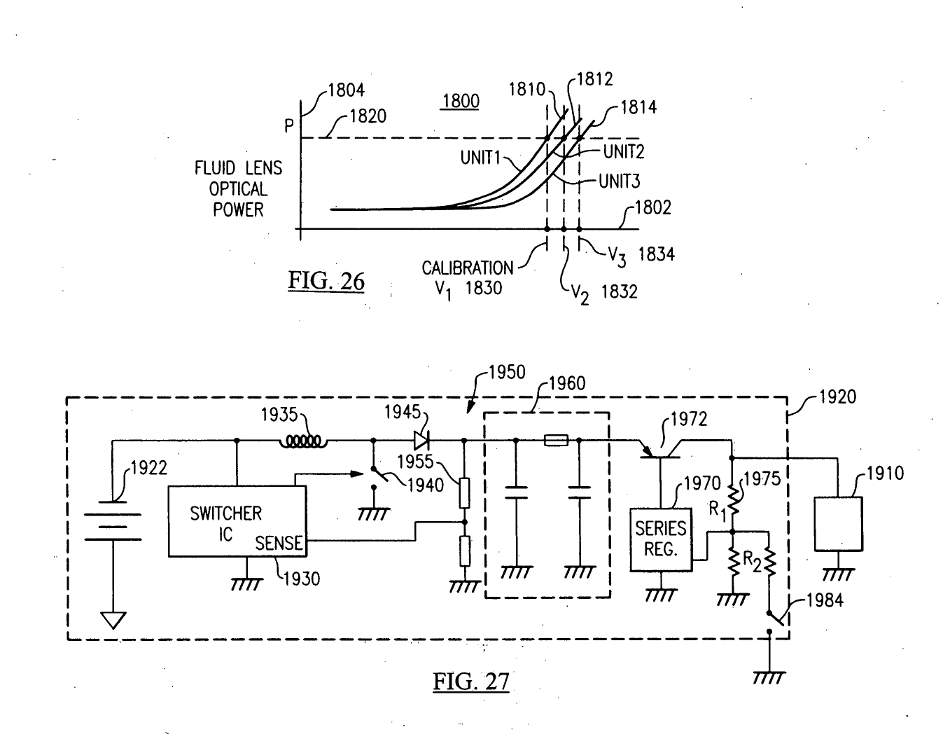

FIG. 34A is a schematic diagram of an alternative embodiment of a fluid lens 2600, and FIG. 34B is a schematic diagram of an alternative embodiment of a distributor module 2640. In FIG. 34A, there are shown a designed number of symmetric connect points on ring 2610, coupled with a continuous ring 2620. In use, a distributor module 2640 will select a pair of connect points, for example 2612c and 2612i, according to the vector (dh, dv) to apply a tilt voltage tv to the pair of connect points 2612c and 2612i that are disposed symmetrically about a center 2630 of the fluid lens. The voltage signals that will be applied are (Vf+tv, Vf−tv). The tilt voltage tv is a function of (dh, dv) and can be predetermined by a mathematical formula or a lookup table. By selecting a material having suitable conductivity (or resistivity) for the ring 2610, the voltage can be made to drop uniformly from point 2612c to point 2612i along the ring 2610 such that a voltage gradient is created to control a fluid lens having a continuously tilt along the direction of (dh, dv). In principle, the resistivity of the material should be high, so that there is not an appreciable current flowing in the ring 2610, to minimize heating and to permit a low power power supply or battery to be used. The ring could be produced by applying a thin layer of conductive material on a nonconductive substrate that is prepared with a desired cross sectional shape. For example, one could build a plastic ring 2610 having an inner diameter, and as appropriate, a taper or other shaped surface to match a design criterion, and then coat the surface intended to lie adjacent the fluid with a thin layer of a highly resistive conductor, such as carbon or tantalum, which are commonly used as thin film resistors. Since there is an insulating layer disposed between the conductor and the fluid in any event, the insulating layer could additionally provide mechanical protection for the thin conductive layer.

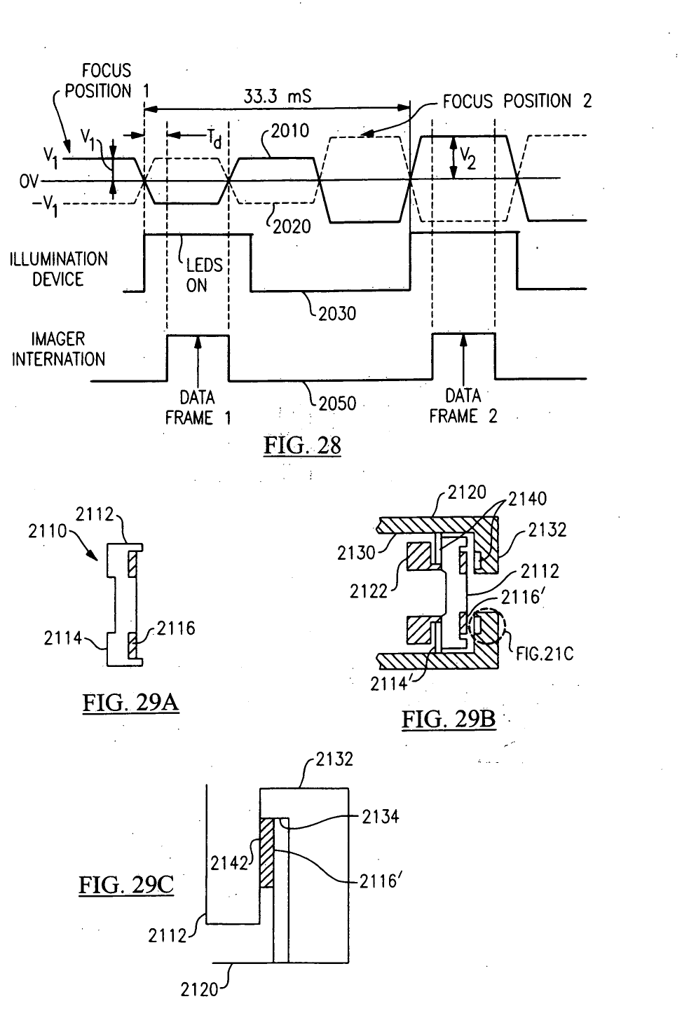

FIG. 35 is a schematic diagram showing the relationship between a fluid lens 2700 and a pair of angular velocity sensors. In a preferred embodiment, two of the angular velocity sensors 2710, 2720 can be integrated with the fluid lens 2700 to form an integrated module 2730. The angular velocity sensors 2710 and 2720 are arranged in an orthogonal relationship to detect two orthogonal angular velocities. In some embodiments, the entire control circuitry as shown in FIG. 33 can also be integrated into the module 2730. An advantage of this embodiment is ease of mounting the module 2730. No vertical or horizontal alignments are required. The module will automatically adjust the lens tilt angle according to the output voltages dh and dv provided by the angular velocity sensors 2710 and 2720.

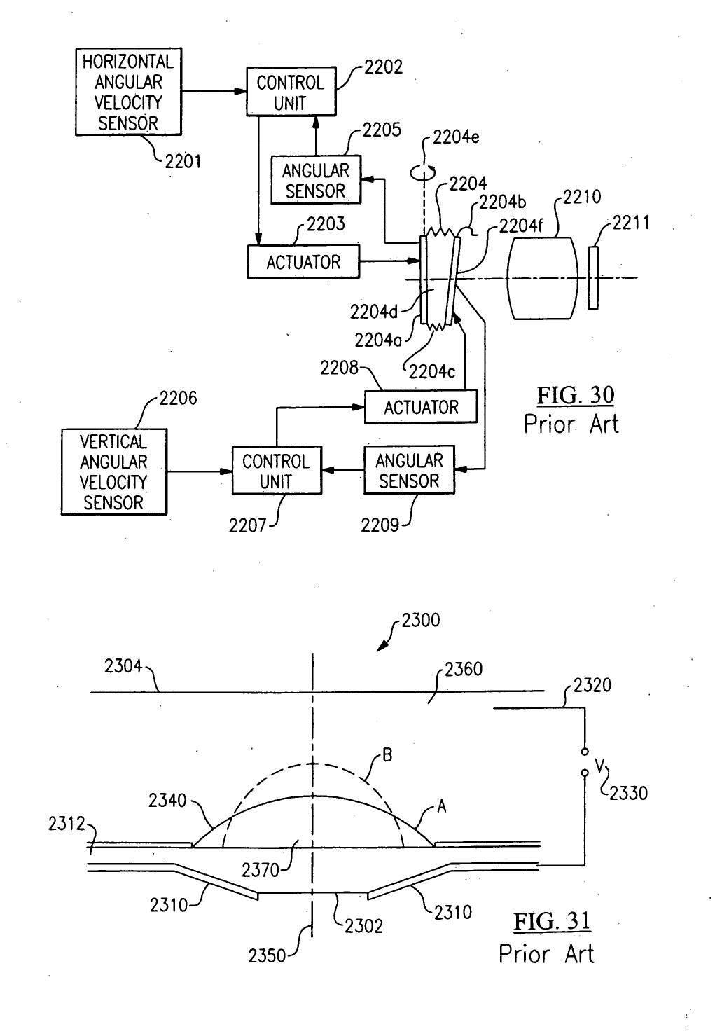

FIG. 36A through FIG. 36E are cross-sectional diagrams of another prior art fluid lens that can be adapted for use in an endoscope. FIG. 36A is a cross-sectional view of a prior art fluid lens having no control signal applied thereto and exhibiting divergence of transmitted light. FIG. 36B is a cross-sectional view of a prior art fluid lens having a control signal applied thereto and exhibiting convergence of transmitted light. FIG. 36C, FIG. 36D, and FIG. 36E are cross-sectional images of fluid lenses having convex, flat and concave interface surfaces as viewed from a position above each lens, respectively.

Descriptions of the Six Applications

Fluid lens systems that operate using voltage signals as the control signal typically involve a first insulating fluid and a second conductor fluid that are in contact at a contact region and are situated within a dielectric chamber. In one embodiment, the insulating fluid and the conductor fluid are both transparent, not miscible, have different optical indexes and have substantially the same density. In some embodiments, the dielectric chamber naturally has a low wetting with respect to the conductor fluid. In such instances, the location of one or both fluids under conditions of no applied voltage can be controlled using a variety of methods, such as applying a surface treatment, or shaping the walls of the chamber. A surface treatment that increases the wetting of the wall of the dielectric chamber with respect to one of the conductor fluid or the insulating fluid and the wall of chamber can serve to define a relative position of an interface between the two fluids.

In another system, according to Berge, the surface treatment is applied to a flat surface comprising the bottom of a container holding the two fluids, and maintains the positioning of a drop of insulating fluid relative to a larger quantity of conducting fluid, preventing the insulating fluid from spreading beyond the desired contact surface. When the system is at rest, the insulating fluid naturally takes a first shape. An optical axis is perpendicular to the contact region between the first and second fluids and passes through the center of the contact region. At rest, the insulating fluid is centered about the optical axis of the device. The elements of the device which are adjacent to the optical axis are transparent. In one embodiment, a transparent first electrode, that transmits light in the vicinity of the optical axis, is placed on the external surface of the wall of the dielectric chamber, on which is situated the insulating fluid. A second electrode contacts the conductor fluid. The second electrode may be immersed in the conducting fluid, or be a conductor deposited on an internal wall of the dielectric chamber. When a voltage V is established between the first and second electrodes, an electrical field is created which, according to the electrowetting principle, changes the wetting properties of the conductive fluid on the bottom surface of the container relative to the nonconductive fluid, so that the conductor fluid moves and deforms the insulating fluid. Because the shape of the interface between the two fluids is changed, a variation of the focal length or point of focus of the lens is obtained.

In alternative embodiments, the two fluids can be present in similar volumes, the interface between one fluid and the other fluid defining a closed curve on the inside wall of a chamber or tube in which the fluids are situated, for example with the inner surface of the cylinder treated, for example by dip-coating, with a suitable surface layer. In alternative embodiments, a first plurality of electrodes can be substituted for the first electrode, and/or a second plurality of electrodes can be substituted for the second electrode, so that a field intensity and a direction of an applied electric signal can be controlled by applying different voltages to two or more of the first plurality of electrodes and/or to two or more of the second plurality of electrodes. In some embodiments, the electrodes can be provided in different shapes, so as to allow different field intensities and directions to be attained by applying a fixed voltage to different ones of the first plurality of electrodes and to different ones of the second plurality of electrodes. In some embodiments, the second electrode, whether or not transparent, is annular in shape, having an open region adjacent an optical axis, so as not to interfere with light passing along the optical axis.

In one embodiment, using a device comprising a fluid lens, an image sensor, and a suitable memory, it is possible to record a plurality of frames that are observed using the fluid lens under one or more operating conditions. The device can further comprise a computation engine, such as a CPU and an associated memory adapted to record instructions and data, for example for processing data in one or more frames. The device can additionally comprise one or more control circuits or control units, for example for controlling the operation of the fluid lens, for operating the image sensor, and for controlling sources of illumination. In some embodiments, there is a DMA channel for communicating data among the image sensor, the CPU, and one or more memories. The data to be communicated can be in raw or processed form. In some embodiments, the device further comprises one or more communication ports adapted to one or more of hard-wired communication, wireless communication, communication using visible or infra-red radiation, and communication employing networks, such as the commercial telephone system, the Internet, a LAN, or a WAN.

In this embodiment, by applying suitable selection criteria, one can use or display only a good frame or alternatively a most suitable frame of the plurality for further data manipulation, image processing, or for display. A device can obtain a plurality of frames of data, a frame being an amount of data contained within the signals that can be extracted from the imager in a single exposure cycle. The device can assess the quality of each of the frames against a selection criterion, which can be a relative criterion or an absolute criterion. Examples of selection criteria are an average exposure level, an extremum exposure level, a contrast level, a color or chroma level, a sharpness level, a decodability level of a symbol within a frame, and a level of compliance of an image or a portion thereof with a standard. Based on the selection criterion, the device can be programmed to select a best or a closest to optimal frame from the plurality of frames, and to make that frame available for display, for image processing, and/or for data manipulation. In addition, the operating conditions for the device can be monitored by the control circuit, so that the conditions under which the optimal frame was observed can be used again for additional frame or image acquisition.

In alternative embodiments, it is possible to use the plurality of frames as a range finding system by identifying which frame is closest to being in focus, and observing the corresponding focal length of the fluid lens. In such an embodiment, the fluid lens can be operated so as to change its focal length over a range of focal lengths, from infinity to a shortest focal length. The device can obtain one or more frames of data for each focal length that is selected, with the information relating to each focal length being recorded, or being computable from a defined algorithm or relationship, so that the focal length used for each image can be determined. Upon a determination of an object of interest within a frame (or of an entire frame) that is deemed to be in best focus from the plurality of frames, the distance from the device to the object of interest in the frame can be determined from the information about the focal length setting of the fluid lens corresponding to that frame. In some instances, if two adjacent frames are deemed to be in suitable focus, the distance may be taken as the average of the two focal lengths corresponding to the two frames, or alternatively, additional frames can be observed using focal lengths selected to lie between the two adjacent frames, so as to improve the accuracy of the measurement of distance.

In another embodiment, apparatus and methods are provided to counteract changes in the environment that surrounds an apparatus comprising a fluid lens. In one embodiment, the apparatus additionally comprises a temperature sensor with a feed back (or feed forward) control circuit, to provide correction to the fluid lens operating signal as the temperature of the fluid lens (or of its environment) is observed to change.

Feedback systems rely on the principle of providing a reference signal (such as a set point) or a plurality of signals (such as a minimum value and a maximum value for a temperature range) that define a suitable or a desired operating parameter (such as a temperature or a pressure), and comparing a measured value of the parameter to the desired value. When a deviation between the observed (or actual) parameter value and the desired parameter value is measured, corrective action is taken to bring the observed or actual value into agreement with the desired parameter value. In the example of temperature, a heater (such as a resistance heater) or a cooling device (such as a cooling coil carrying a coolant such as water) can be operated to adjust an actual temperature. Using a feedback loop, the apparatus is made to operate at the desired set point, or within the desired range. Feedback loops can be provided using either or both of digital and analog signal processing, and using one or more of derivative, integral and proportional (“D-I-P”) controllers.

In some embodiments, a feed-forward system can be used, in which a change (or a rate of change) of a parameter such as actual or observed temperature is measured. Corrective action is taken when it is perceived that a condition outside of acceptable operating conditions likely would be attained if no corrective action were to be applied and the observed change (or rate of change) of the parameter were allowed to continue unabated for a further amount of time. Feed-forward systems can be implemented using either or both of digital and analog signal processing. In some systems, combinations of feedback and feed-forward systems can be applied. In some embodiments, multiple feedback and feed-forward controls can be implemented.

In the embodiment contemplated, the operating parameter, such as temperature, of the apparatus comprising a fluid lens, or of the environment in which it is situated, is monitored, and the observed parameter is compared to one or more pre-defined values. The one or more predefined values may be fixed (such as a maximum tolerable temperature above which a substance begins to degrade at one atmosphere of pressure) or the one or more predefined values may depend on more than one parameter, such as the combination of pressure and temperature, for example using relationships in a pressure-temperature-composition phase diagram (for example, that a substance or chemical composition in the fluid lens apparatus undergoes a phase change if the pressure and temperature vary such that a phase boundary is crossed, or undergoes a change from covalent to ionic character, or the reverse).

In yet another embodiment, a system comprising a fluid lens additionally comprises a non-adjustable lens component configured to correct one or more specific limitations or imperfections of the fluid lens, such as correcting for color, spherical, coma, or other aberrations of the fluid lens itself or of the fluid lens in conjunction with one or more other optical components. By way of example, a fluid lens may exhibit dispersive behavior or color error. In one embodiment, a second optical element is added that provides dispersion of the sign opposite to that exhibited by the fluid lens, so as to correct the dispersive error introduced by the fluid lens. In one embodiment, the dispersive element is a diffraction element, such as an embossed grating or an embossed diffractive element. As will be understood, different optical materials have different dispersive characteristics, for example, two glass compositions can have different dispersion, or a composition of glass and a plastic material can have different dispersion. In the present invention, a material having a suitable dispersive characteristic, or one made to have suitable dispersive characteristics by controlling the geometry of the material, such as in a grating or other diffractive element, can be used to correct the errors attributable to the fluid lens and/or the other components in an optical train.

The aberrations that are possible in a fluid lens can in principle be of any order, much as the aberrations that are possible in the lens or the cornea of a human eye. Both a human eye and a fluid lens operate using interfaces between two or more dissimilar fluids. In the human eye, there are membranes that are used to apply forces to the fluids adjacent the membranes, by application of muscle power controlled by signals created by the nervous system. In a fluid lens, there are forces that are applied, in some instances to the fluid or fluids directly by electromagnetic signals, and in some instances by forces applied to transparent membranes that are adjacent the fluids. Both kinds of systems can be affected by external forces, such as the force of gravity and other accelerative forces, changes in ambient or applied pressure, and changes in ambient or applied temperature.

In still another embodiment, there is provided a calibration tool, process, or method for calibrating a fluid lens. As one example, a system comprising a fluid lens is operated at one or more known conditions, such as one or more magnifications or one or more focal lengths. For each known operating condition, an operating parameter, such as a value of the driving voltage, is observed or measured. The observed or measured data is stored in a memory. The data in memory is then used to provide calibration data for application to the operation of the fluid lens.

Even if two or more nominally identical fluid lenses are provided, there can be differences that exist in the two fluid lenses themselves, as has been explained hereinbefore. When intrinsic differences between two nominally identical fluid lenses exist, application of a substantially identical fluid lens control signal to the two lenses can result in different operative behavior for each lens. A default calibration can be provided, for example based on a calibration performed under controlled or defined conditions. The default calibration data can be recorded and used at a later time to operate the fluid lens for which the calibration was obtained. Using such calibrations is an effective and efficient way to operate a given fluid lens over a defined operating range. For many purposes, such information is well worth having and helps to provide a fluid lens that is conveniently operated in a predictable manner. Between calibration points, interpolation can be used to achieve an improved resolution. Similarly extrapolation may be used to estimate the attributes of a feature beyond the range of measured calibration data.

In addition, as has been indicated, differences may be externally imposed, such as applied voltage, ambient or applied pressure, ambient or applied temperature, and accelerative forces. These forces may, individually and in combination, cause one fluid lens to operate somewhat differently than a nominally identical fluid lens. When such differences in operating conditions exist, application of a substantially identical fluid lens control signal to the two lenses can result in different operative behavior for each lens. Accordingly, it can be helpful to provide a simple and readily applied calibration method for a fluid lens, so that each lens can be calibrated and provided with suitable fluid lens control signals to operate in a desired fashion under the particular conditions pertaining to that fluid lens.

Yet another reason for providing calibration capabilities relates to changes in operation of a given fluid lens over time. The operation of an individual fluid lens relies on one or more of the chemical, mechanical, and electrical properties of the components of the fluid lens, which properties may change with time and with use. For example, as indicated hereinabove, a fluid lens operating in response to electrical signals may undergo electrochemically driven reactions in one or more fluids. In addition, a fluid may change properties over time as a result thermal history, such as of repeated heating and cooling cycles or exposure to extremes of temperature. As will be understood, as a property of one or more components of a fluid lens changes with time, it may be advantageous to calibrate the operating conditions of interest.

In still a further embodiment, an inertial device such as an accelerometer is provided to determine an orientation of a fluid lens, which orientation information is used to self-calibrate the fluid lens. Gravitational and other accelerative forces can cause fluids to move and change shape at a free boundary, or a boundary where two fluids come into mutual contact. By way of example, consider a fluid lens that comprises two fluids having slightly different densities. Different density implies that equal volumes of the two fluids will have proportionately different masses, because density=mass/volume. Therefore, since Force (F)=mass×acceleration, the equal volumes of the two fluids will experience slightly different forces under equal acceleration, such as the acceleration of gravity, or of an external accelerative force applied to a container holding the two fluids. One consequence of such an applied acceleration can be a change in the relative locations of the fluids, and as a result, a change in the shape of the interface defined by the surface of contact between the two fluids. In addition, the direction of application of the acceleration will also have a bearing on the response of the fluids. For example, an acceleration applied normal to a flat interface between the two fluids may have much less of an effect than an acceleration parallel to, or tangent to, a surface component of the interface between the two fluids. Since the accelerative force in general can be applied at any angle with regard to an interface between the two fluids, there will in general be differences in response depending on the precise orientation of the applied accelerative force. Inertial sensors such as accelerometers and gyroscopes can be useful in determining and in tracking the position of an object over time. Through the use of such inertial sensors, it is possible to discern an orientation of an object, and to measure the magnitudes and directions of applied accelerative forces. It is possible to calculate or to model how the fluids present in the lens will respond to the forces operating on the lens with knowledge of the orientation of a fluid lens and of the external forces, including that of gravity. While the description presented hereinabove may be understood to describe linear accelerative forces such as gravity, it is also possible to perform both the tracking and the calculation of the responses of fluids to forces having non-linear components, forces having rotational components, or time-varying forces. In some embodiments, using appropriate sensors for various forces, one can determine the relative orientation of the applied force and the interface between two fluids, and compute what response would be expected. As a result of the computation, information is provided for the timely application of restorative forces. For example, by modifying the magnitude and/or the field direction of an electrical signal, if necessary as a function of time, the expected distortion of the fluid interface can be counteracted. In one embodiment, solid state accelerometer sensors are provided that operate at sufficiently high rates as to determine the magnitude and orientation of an external force. Accelerometers having response rates of at least 10,000 Hz are available from Crossbow Technology, Inc. located at 4145 N. First Street, San Jose, Calif. 95134.

In yet an additional embodiment, in an apparatus comprising a fluid lens, the fluid lens is operated to provide corrective properties with regard to such distortions as may be caused by vibration, location or orientation of the lens, chromatic aberration, distortions caused by higher order optical imperfections, and aberrations induced by environmental factors, such as changes in pressure. As has been explained hereinbefore, using accelerative forces as an example, the fluid lens may in some instances be subjected to various distorting forces or to forces that cause degradation of the operation of the fluid lens from that which is desired. In other instances, the fluid lens may have inherent imperfections, such as chromatic aberration or higher order optical imperfections. It is possible to analyze such optical imperfections in various ways, such as the use of a calibrated imaging system comprising a source, at least one image sensor, and hardware and/or software configured to analyze optical information to assess whether errors or imperfections exist in an optical component under test. The calibrated imaging system in some instances can be a laboratory setting in which highly sophisticated equipment is employed to perform tests. In other instances, the calibrated test system can comprise a source that provides a known optical signal that is passed through an optical component under test, and the analysis of the resulting signal that emerges from the optical component under test. The calibrated test system in some embodiments is a system or device suitable for use in the field, so that periodic calibration can be performed in a convenient and efficient manner, if necessary by personnel who are not familiar with all of the sophistications of optical testing in a laboratory setting.

In one embodiment, the optical component can be modeled in the frequency domain as a transfer function, wherein a known applied input signal I(s) is provided and an observed output signal O(s) is measured. An observed transfer function H_{obs}(s)=O(s)/I(s) is determined. H_{obs}(s) can then be compared to a desired transfer function H(s), to determine a corrective factor or relation C(s) that should be applied to the system under test to cause it to perform as desired, where C(s)H_{obs}(s)=H(s), or C(s)=H(s)/H_{obs}(s). Once the corrective factor or relation C(s) has been determined, it (or its time domain equivalent) can be applied to drive the fluid lens so as to reduce the observed imperfection or imperfections. Transfer function concepts, discrete time mathematical procedures, digital filters and filtering methods, and circuitry (including hardware and software) that can handle the required detection, analysis and computation, and can be used to apply corrective action are described in many texts on real time digital signal processing. Hardware such as digital signal processors are commercially available from multiple vendors.

Applications for fluid lenses include their use in one or more types of camera, such as cameras in cell phones, use in higher quality digital cameras such as those having a high powered zoom lens, and use in cameras that can provide autofocus, and pan, tilt, and zoom (“PTZ”). Panning is moving a camera in a sweeping movement, typically horizontally from side to side. Tilting is a vertical camera movement, e.g. in a direction orthogonal to panning. Commercially available PTZ video and digital cameras that use mechanical redirection of the camera and refocusing of its lens are well known, and are often used in surveillance. In order to accomplish such features as tilt or pan, one needs to reorient the interface between two optically dissimilar fluids so that the optical axis is relocated from its original direction horizontally (pan) or is relocated from its original direction vertically (tilt). With a fluid lens, both relocations can be accomplished in a single redirection of the optical axis at an angle to both the horizontal and vertical directions simultaneously. Such redirections are readily computed using spherical geometry coordinates, but can also be computed in any coordinate system, including using projection from three dimensions to two dimensions, for example as is commonly done in x-ray crystallography as an example. One method to accomplish all of autofocus, pan, tilt, and zoom is to apply several features in a single device. Autofocus and zoom have been addressed hereinbefore. Pan and tilt, or more generally, redirection of the optical axis to a new orientation that is non-collinear with the original optical axis, can be accomplished by providing an electrode pair comprising a first plurality of first electrodes and at least one second electrode, and applying voltages to at least one electrode of the first plurality and the at least one second electrode so that the surface shape of the interface between the two fluids in the fluid lens is caused to change a measure of asymmetry as measured with respect to the optical axis of the fluid lens prior to the application of the voltages. In general, to accomplish the provision of an asymmetry, either the applied voltages will include an asymmetric component, or the electrodes to which the voltages are applied will be positioned in an asymmetric geometrical relationship, or both. By applying a voltage field having an asymmetry to the fluids in the fluid lens, the fluids will respond in a manner to adjust the voltage gradients across the interface to be as uniform as possible, thereby causing the fluids to take up an interface shape that comprises an asymmetric component, and thereby directing light along a new optical axis that is non-collinear with the optical axis that existed prior to the application of the voltage.

We will now briefly describe examples of power supplies that are useful for powering a fluid lens. In one embodiment, a suitable power supply for driving the fluid lens is a square wave power supply that is biased to operate in the range 0 to V volts, where V is either a positive or a negative voltage, which may be thought of as a unipolar supply. One embodiment is to use a bipolar power supply that is capable of providing voltages between +V₁/2 and −V₁/2 volts, with an added bias voltage of +V₁/2 volts (causing the range to extend from 0 volts (=+V₁/2 volts bias +[−V₁/2 volts] supply) to +V₁ volts (=+V₁/2 volts bias+V₁/2 volts supply), or alternatively using an added bias voltage of −V₁/2 volts (causing the range to extend from −V₁ volts (=−V₁/2 volts bias +[−V₁/2 volts] supply) to 0 volts (=−V₁/2 volts bias+V₁/2 volts supply). The summation of two voltages is easily accomplished with a summing circuit, many variations of which are known. In one embodiment, the bias voltage supply operates at a fixed voltage. In other embodiments, the bias voltage supply is configured to provide a plurality of defined voltages, based on a command, which may be provided by setting a switch, or under the control of a microprocessor. In some embodiments, voltage supplies are used that can be controlled by the provision of a digital signal, such as a digital-to-analog converter controlled by a digital code to define an output signal value. In another embodiment, voltage supplies that are controlled using a frequency-to-voltage converter, such as the National Semiconductor LM2907 or LM 2917 frequency-to-voltage converter, can be employed using a pulse train having a controllable frequency as a control signal. It is believed that electrochemical effects within the fluid lens are operative under sufficiently high applied voltages, thereby making the use of a unipolar supply advantageous in some instances.

In other embodiments, power supplies that provide voltage signals having both positive and negative peak voltages of the order of one volt to hundreds of volts are provided. In some embodiments, the output voltages are provided as square waves that are generated by a driver integrated circuit such as is commonly used to operate electroluminescent lamps, such as are found in cellular telephones.

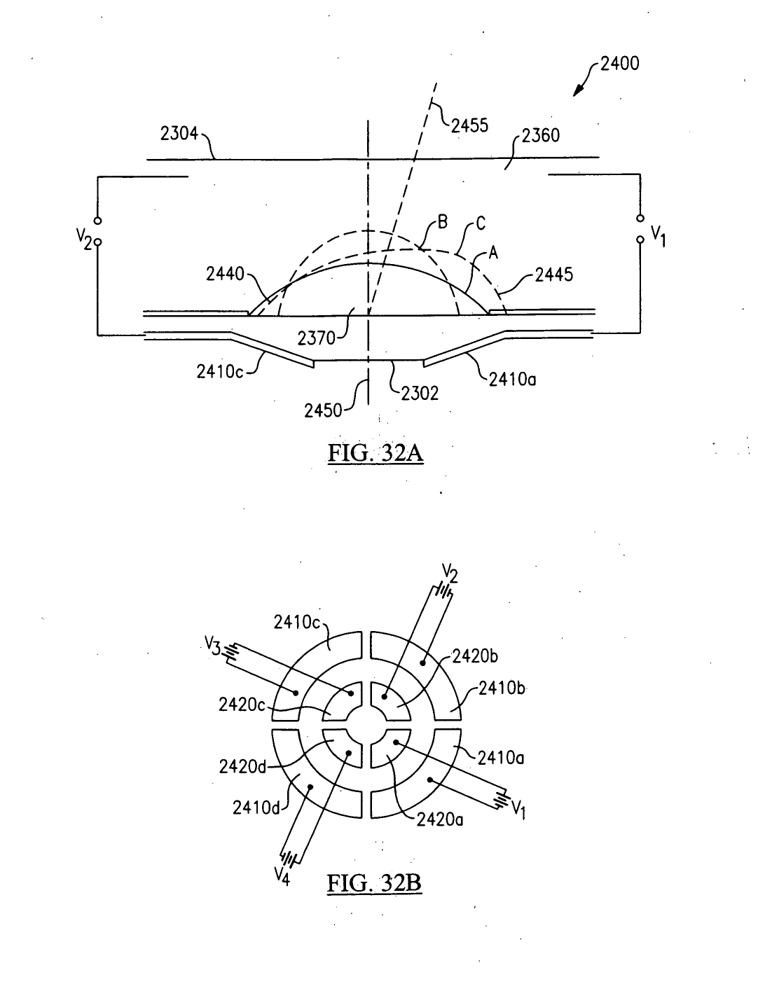

FIG. 37 is a schematic block diagram showing an exemplary fluid lens driver circuit 2900. The circuit is powered by a battery supply 2910, typically operating in the range of 3 to 4.5 volts, although circuits operating with batteries of other voltages and also operating from fixed wall mount power supplies can be designed. A voltage reference 2920 is provided which may have associated with it a low drop out voltage regulator. Input signals in the form of a clock signal (a frequency or a pulse train) and digital data line are provided to an I²C serial interface 2930 for control of this driver circuit by an external device, such as the microprocessor 1040 of FIG. 10. The serial interface 2930 is in communication with a controller 2940 (such as a commercially available microcontroller) for coordinating the activities of the fluid lens driver circuit 2900, the oscillator 2960, to set the output frequency, and a digital-to-analog (DAC) converter 2950, to set the output voltage. The DAC is provided with a reference voltage by the voltage reference 2920. In some embodiments the DAC is a 10 bit DAC.

The controller 2940 is in communication with an oscillator 2960 that provides a timing signal. This oscillator 2960 can be signaled to enter a power down state by a suitable signal communicated from an external source at 2962, which in some embodiments can be a user or can be another controller. The controllers contemplated herein are in general any microprocessor-based controller including a microcontroller, a microprocessor with associated memory and programmed instructions, or a general purpose digital computer. The controller 2940 is also in communication with a wave form generator 2945 that creates the square wave waveform for the bridge driver output stage 2980. The waveform generator 2945 also synchronizes the DAC transitions with the output waveform through the controller 2940.

The output of the DAC 2950 sets the output voltage level of the high voltage generator 2970 such that the output voltage is proportional to the output of the DAC 2920, and thereby is configured to be controlled with high precision by a digital source such as a computer. In some embodiments, appropriate feedback circuitry is contained in this portion of the circuit to keep the output voltage constant over a range of input voltage, load and environmental conditions. The high voltage created by the high voltage generator 2970 is an input to the bridge driver 2980. The high voltage generator has a stable output ranging from 0 Volts to approximately 40 Volts for the Varioptic ASM-1000 fluid lens. This generator may utilize an inductor 2972 and or capacitors to create the higher voltage. However other circuit configurations might also be used, for example capacitive voltage multipliers. The bridge driver 2980 creates the high voltage switching signals OUTP and OUTM which drive the fluid lens 2995. In some embodiments, the output can be applied to a load such as fluid lens 2995 using the commutating circuit of FIG. 23.

The output to the fluid lens is a voltage signal that is waveshaped by the bridge driver using a wave form signal from the wave form generator. The term “bridge driver” should be understood as follows. The load is connected between two amplifier outputs (e.g., it “bridges” the two output terminals). This topology can double the voltage swing at the load, compared to a load that is connected to ground. The ground-tied load can have a swing from zero to the amplifier's supply voltage. A bridge-driven load can see twice this swing because the amplifier can drive either the +terminal of the load or the—terminal, effectively doubling the voltage swing. Since twice the voltage means four times the power, this is a significant improvement, especially in applications where battery size dictates a lower supply voltage, such as in automotive or handheld applications.

As already indicated, one can also sum the output of the circuit described with a reference signal of suitable magnitude and polarity so that the voltage swing experienced by the load is unipolar, but of twice the magnitude of either the positive or negative voltage signal relative to ground. The power advantage just referred to is also present in such an instance, because power P is given by the relationship V²/R or V²/Z, where V is voltage, R is resistance, and Z is impedance. Since the voltage swing in both embodiments is the same v volts (e.g., from −v/2 to +v/2, from 0 to +v, or from −v to 0), the power available is unchanged. Stated in terms that will be familiar to those acquainted with the principles of electrical engineering, since the reference voltage of an electrical system (for example ground potential) may be selected in an arbitrary manner, merely shifting the voltages applied to the fluid lens from one reference to a different reference should not change the net power delivered to the fluid lens. However, when considered from the perspectives of electrochemical principles, it is recognized that different electrochemical reactions can be made to occur (or can be suppressed) depending on whether an applied electrical signal is a positive-going, or a negative-going, voltage relative to the reference voltage (e.g., polarity may be an important feature in a particular chemical system).

Use of Fluid Lens in Illumination Systems.

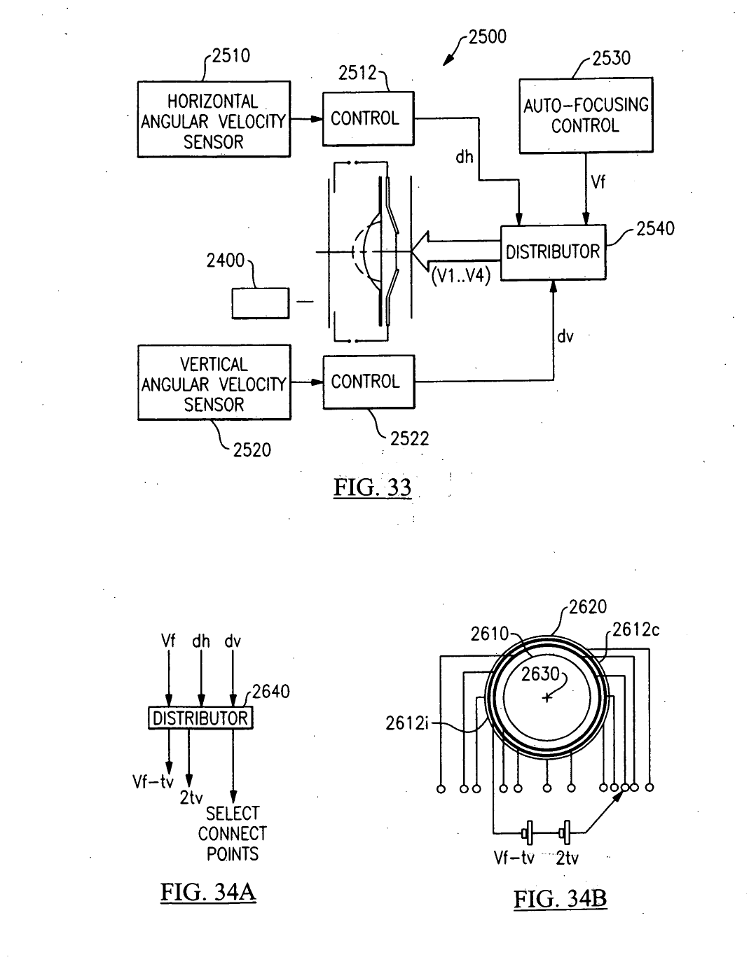

FIG. 38A and FIG. 38 B are diagrams that show an LED die 3010 emitting energy in a forward direction through a fluid lens 3020. The divergence of the emitted light is modified with the fluid lens. In FIG. 38A the divergence of the emitted light is modified because of the optical power of the fluid lens. In the example shown the light exiting the fluid lens could be considered to approximate collimated light even though the light exiting the LED is diverging. In a situation where the curvature of the fluid lens is more extreme than is shown in FIG. 38A, the light may be focused on a smaller region. In FIG. 38B the power of the fluid lens has been reduced to approximately zero so that the divergence of the light emitted by the LED is substantially unchanged. The comparison of the light patterns in FIG. 38A and FIG. 38 B indicates that such systems can be used to control the coverage (in area) at a target of interest, for example an object that one is interested in observing with an endoscope or imager. In some embodiments, one or more windows on an endoscope or scanner may also be used to protect the optical system including the fluid lens from adverse environmental conditions.

It should be appreciated that although the details may change, this concept also applies to encapsulated LEDs, as well as to fluid lens assemblies that may contain additional optical elements such as spherical, aspherical and cylindrical lens elements.

In one embodiment, such a system is expected to more efficiently utilize a higher fraction of light emitted by the LEDs. For example when viewing objects near the imager, a more diverging illumination pattern is desirable in order to be assured that larger features are illuminated over their entire extent and when viewing objects at a larger distance from the imager, a more converging illumination pattern is desirable so that illumination is not wasted by falling outside the optical field of interest.

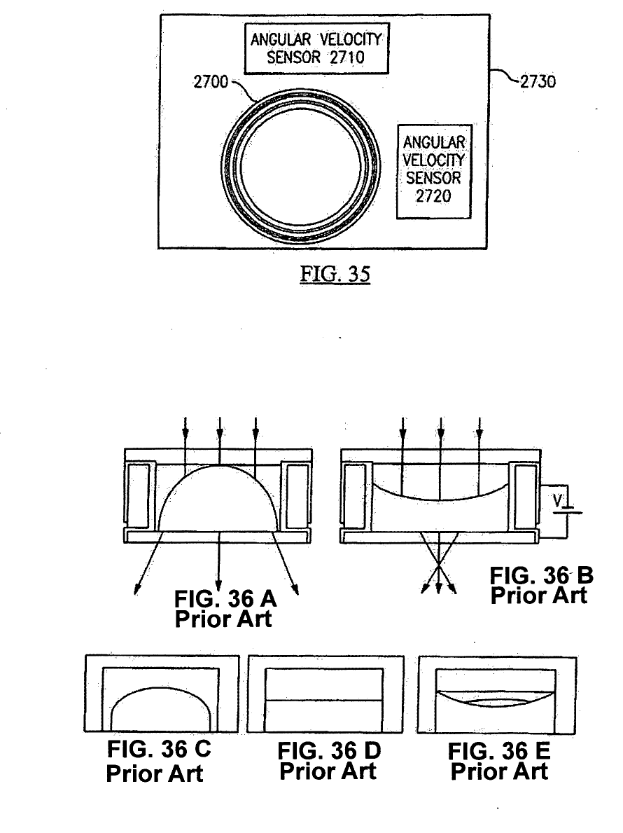

FIG. 39A, FIG. 39B and FIG. 39C show diagrams of a laser scanner comprising a laser 3110, a collimating lens 3120, and a fluid lens 3130 in various configurations. In FIG. 39A the fluid lens is configured to have a first optical power, a first focal length and a first principal beam direction. The light beam emanating from the fluid lens 3130 is focused to have a narrowest beam width at a plane 3140 situated at a first distance D1 from the fluid lens 3130. In FIG. 39B the fluid lens is configured to have a second optical power, a second focal length and a first principal beam direction. In FIG. 39B, the light beam emanating from the fluid lens 3130 is focused to have a narrowest beam width at a plane 3141 situated at a second distance D2 from the fluid lens 3130, such that D2 is greater than D1, and the first principal beam direction is not changed when the focal length of the fluid lens 3130 is changed. In FIG. 39C the fluid lens is configured to have a first optical power, a first focal length and a second principal beam direction. In FIG. 39C, the light beam emanating from the fluid lens 3130 is focused to have a narrowest beam width at a plane 3140 situated at a first distance corresponding to a distance D1 from the fluid lens 3130 measured along the second principal beam direction of FIG. 39A, but because the beam in FIG. 39C is emanating at an angle (e.g., the third principal beam direction is not the same as the first principle beam direction), the lateral distance that the beam is “off-axis” is LI. Other optical powers, focal lengths and principle beam directions can be achieved by properly configuring and energizing the fluid lens 3130.

The present inventions are intended to take advantage of fluid lens zoom optical systems. Fluid Zoom lens configurations can be used in endoscopes to enable imaging of different objects at various distances from the endoscope. In endoscopes manufactured today, often a large working distance is achieved by stopping down the lens aperture to increase the optical depth of field. However this has two disadvantages: First, when the lens stop is smaller, the optical system point spread function increases thereby making it more difficult to scan objects with narrow features. Second, when the lens stop is smaller, less light enters the lens thereby reducing the signal-to-noise ratio of the system. The lower SNR requires the operator to hold the endoscope still for longer period of time. The effect is that the apparatus has an increased sensitivity to hand motion. In addition, because longer periods of time are required, the user is more likely to become fatigued.

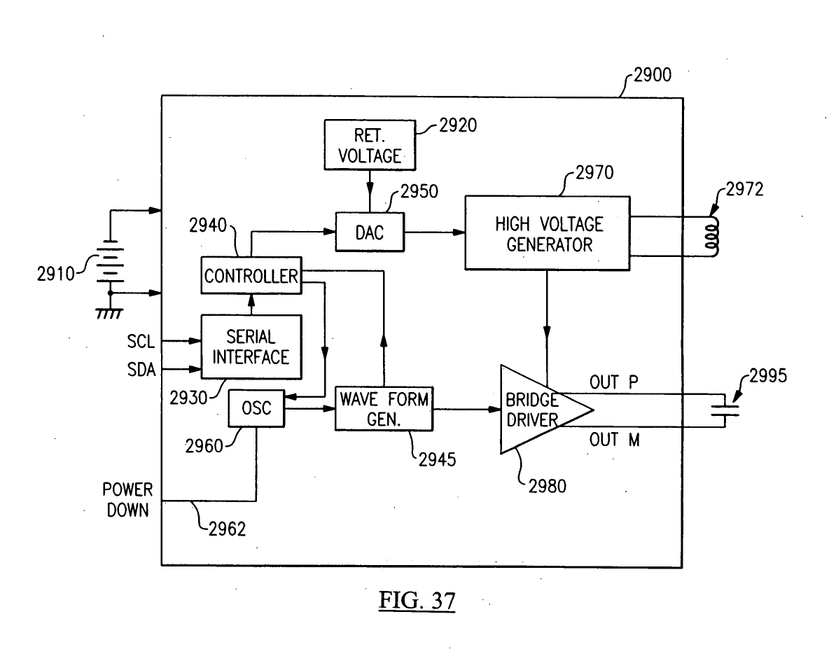

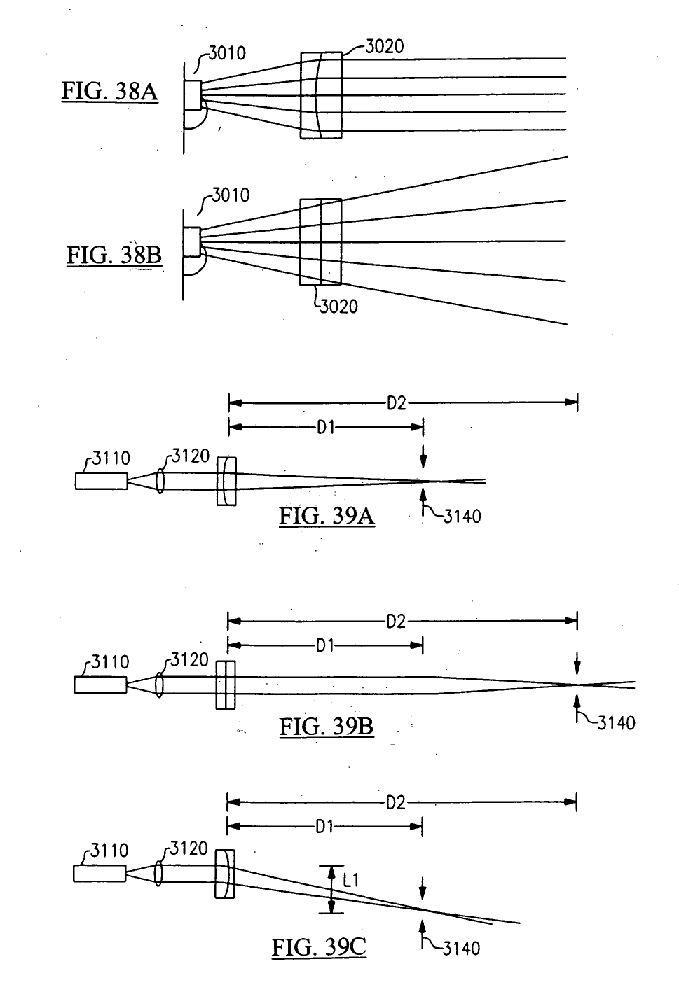

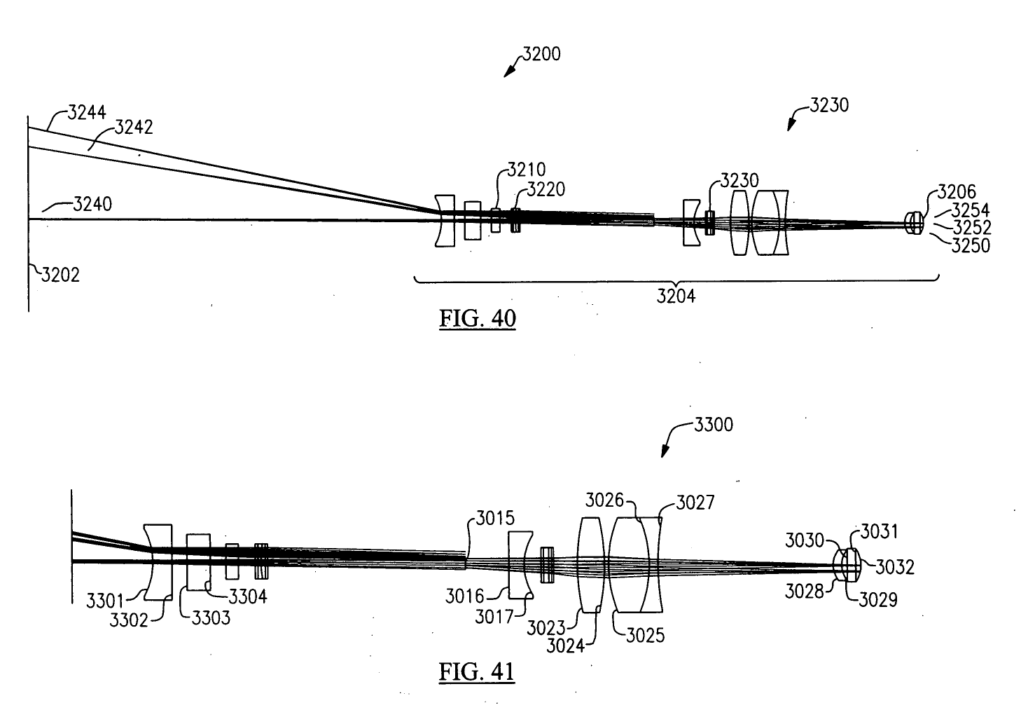

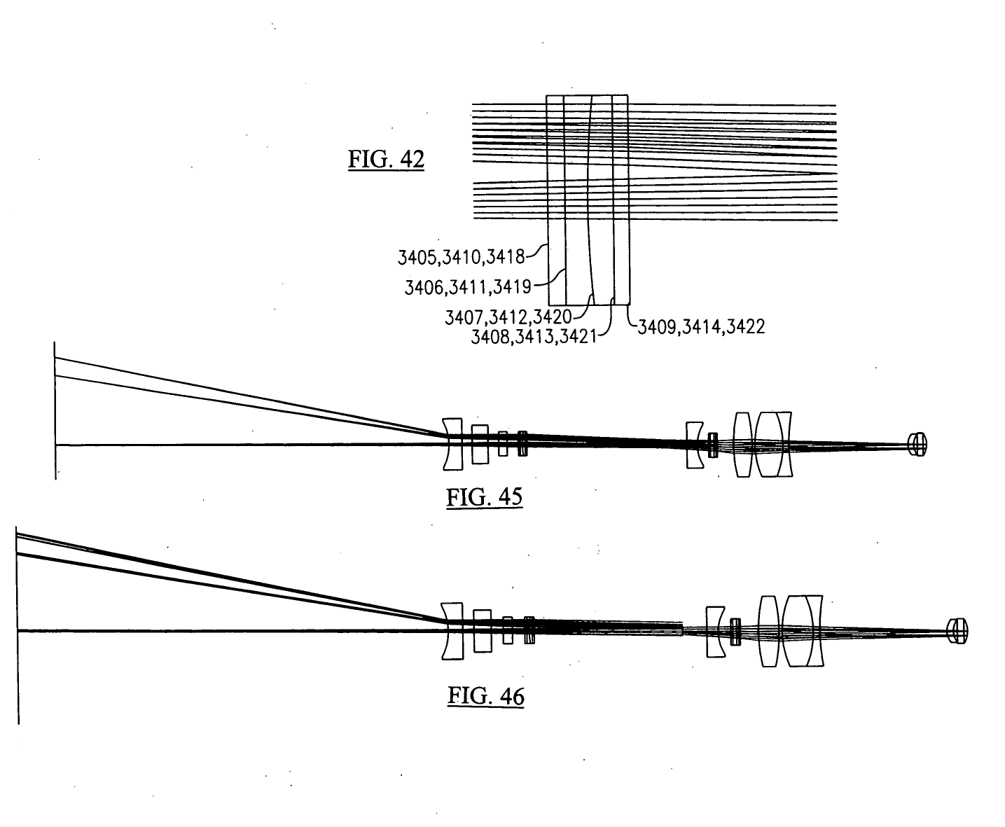

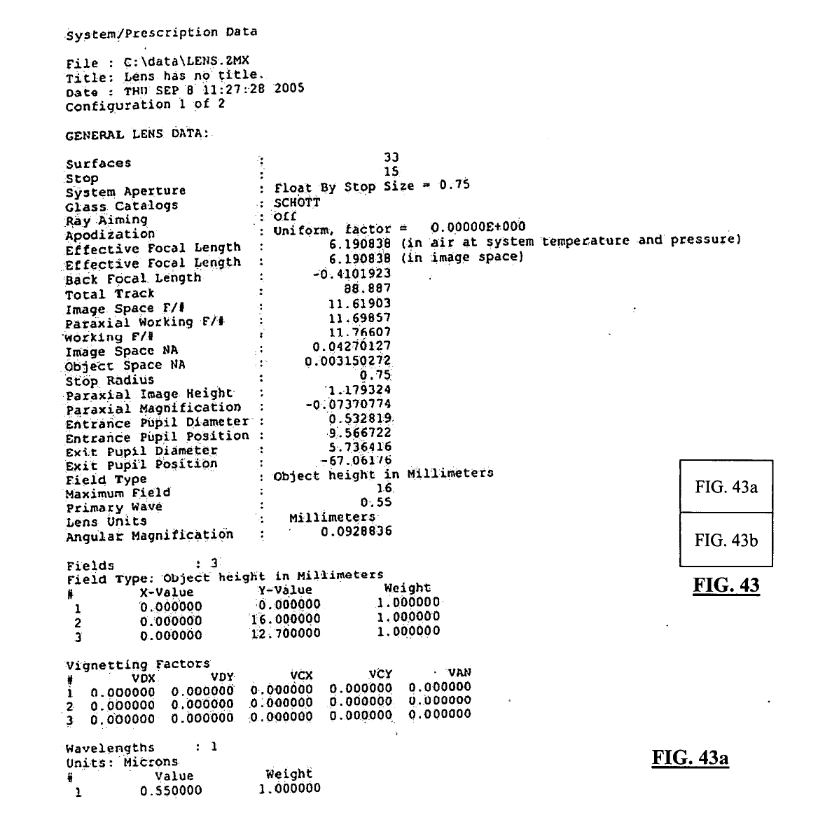

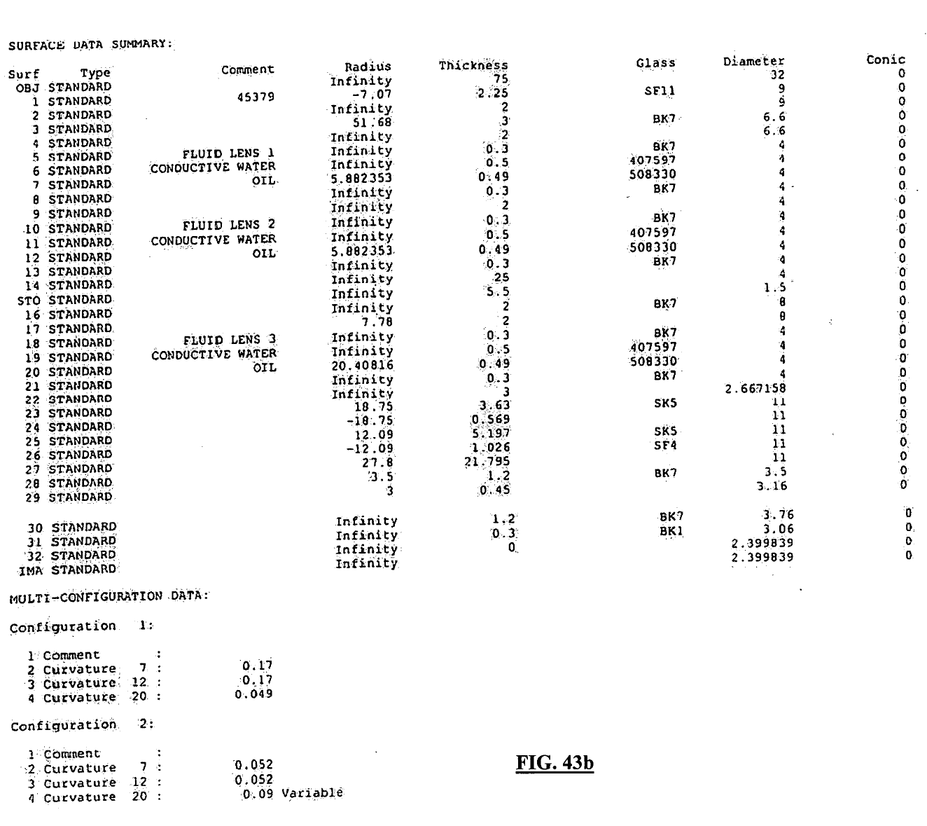

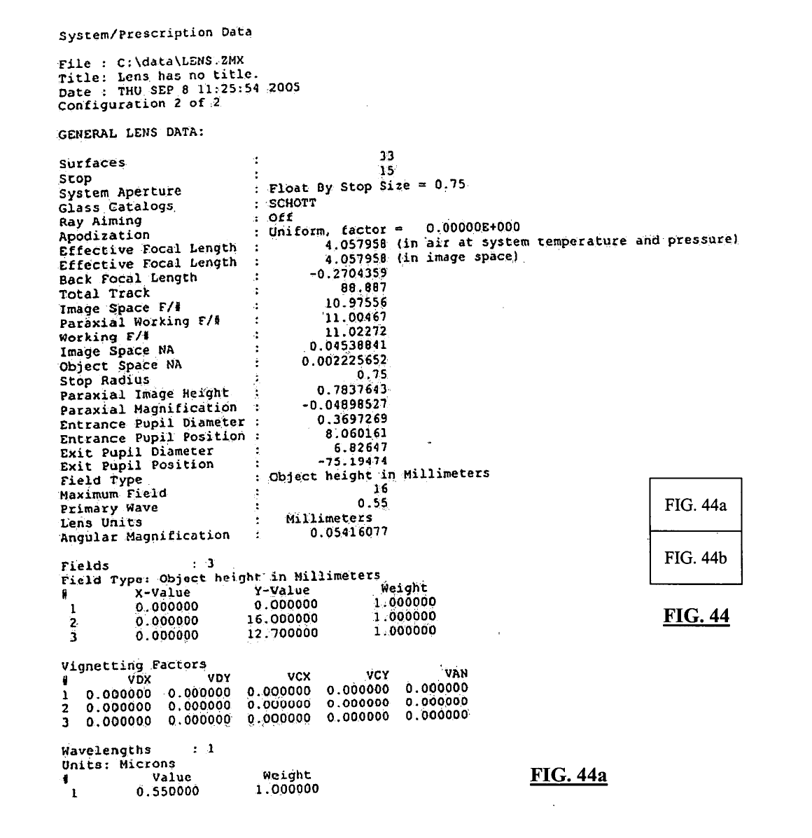

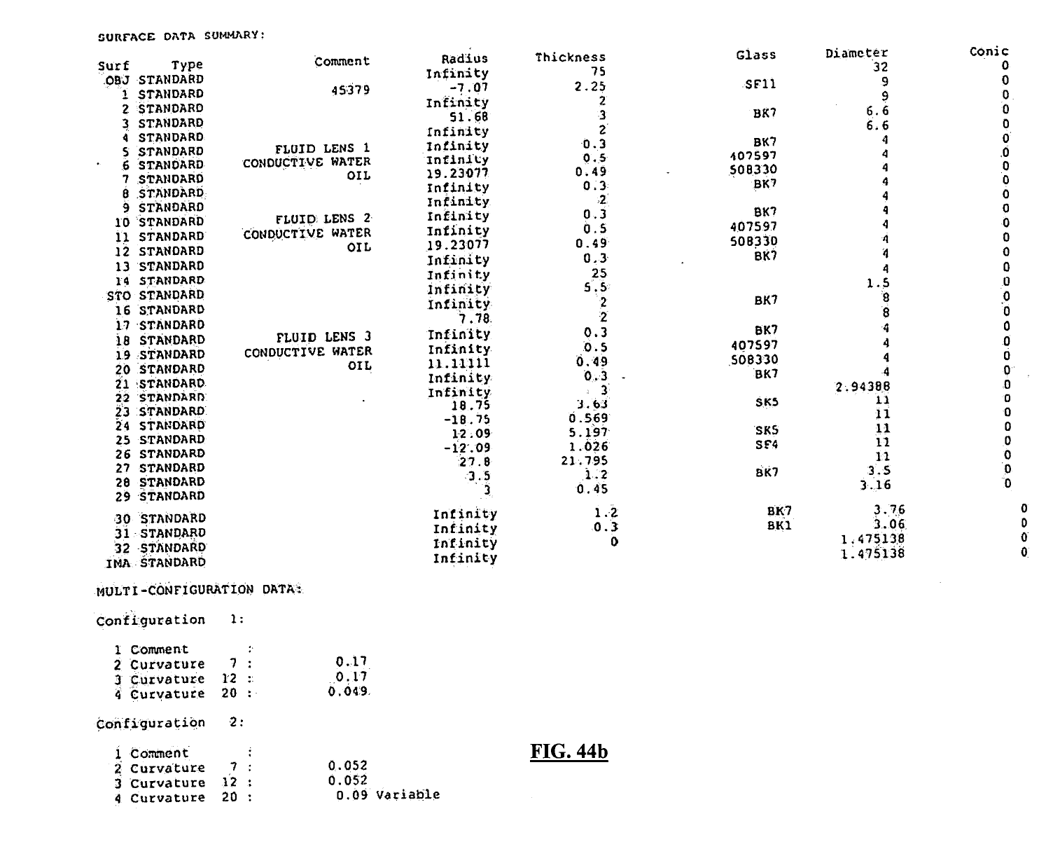

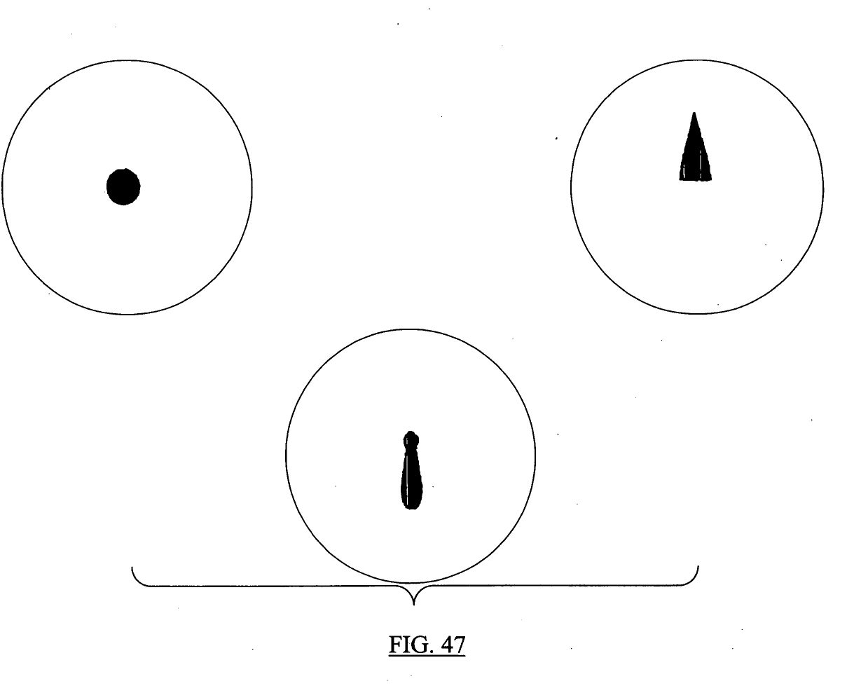

According to one embodiment, a sketch of zoom lens configuration 3200 is shown in FIG. 40. The object 3202 is imaged with lens assembly 3204 onto the image plane 3206. This zoom lens makes use of 3 fluid lenses 3210, 3220 and 3230. The lens system 3200 images three object points 3240, 3242 and 3244 onto the image plane 3206 at the respective points 3254, 3252 and 3250 respectively. Observe that because the image locations are not resolved in this figure, the individual image points cannot be seen. The details of zoom lens 3204 are shown in more detail in FIG. 41 and this figure show each of the lens surfaces called out for all elements except the fluid lens elements that are shown in the detail of FIG. 42. The table below defines the individual optical elements of the zoom lens system 3300 shown in FIG. 41. Note that all 3 zoom lenses are structurally identical in construction and the details of a single fluid lens are shown in FIG. 42 with notation for all 3 fluid lenses. This particular implementation of a zoom lens was modeled at the two end zoom configurations. Other intermediate points could also have been modeled. The optical surface details of the two zoom configurations are shown in the multi-configuration table shown below. The detailed ZEMAX prescriptions for the two configurations are shown in FIG. 43 and FIG. 44 for configurations 1 and 2 respectively. FIG. 45 and FIG. 46 show the complete ray traces for the configurations 1 and 2 respectively and FIG. 47 and FIG. 48 show the image spot sizes for configurations 1 and 2 respectively.

The zoom lens optical configuration shown was made using available materials in an effort to demonstrate feasibility. Two fluid lenses adjacent to each other were used in order to obtain the desired optical power. Other optical zoom lens configurations are also anticipated by this design, including systems using only 2 fluid lens, or more fluid lenses.

All dimensions are given in millimeters unless otherwise specified.

The three object fields are defined below

| Field | Y-Value |
|---|---|
| 1 | 0.000000 |
| 2 | 16.000000 |
| 3 | 12.700000 |

The lens surfaces used are defined in the prescription table shown below. The table is shown for zoom condition 2.

| Surface | Type | Comment | Radius | Thickness | Glass | Diameter |
|---|---|---|---|---|---|---|
| 0 | Object | Object distance | Infinity | 75 | | |
| 1 | Lens | Edmund Scientific | −7.07 | 2.25 | SF11 | 9 |
| | | Lens 45379 | | | | |
| 2 | Air gap | | Infinity | 2 | 9 | |
| 3 | Lens | Lens | 51.68 | 3 | BK7 | 6.6 |
| 4 | Air gap | | Infinity | 2 | | 6.6 |
| 5 | Window | Fluid lens 1 | Infinity | 0.3 | BK7 | 4 |
| 6 | | Conductive water | Infinity | 0.5 | 407597 | 4 |
| 7 | | Oil | 19.23077 | 0.49 | 508330 | 4 |
| 8 | Window | | Infinity | 0.3 | BK7 | 4 |
| 9 | Air gap | | Infinity | 2 | | 4 |
| 10 | Window | Fluid lens 2 | Infinity | 0.3 | BK7 | 4 |
| 11 | | Conductive water | Infinity | 0.5 | 407597 | 4 |
| 12 | | Oil | 19.23077 | 0.49 | 508330 | 4 |
| 13 | Window | | Infinity | 0.3 | BK7 | 4 |
| 14 | Air gap | | Infinity | 25 | | 4 |
| STO | Aperture stop | | Infinity | 5.5 | | 1.5 |
| 16 | Lens | | Infinity | 2 | BK7 | 8 |
| 17 | Air gap | | 7.78 | 2 | | 8 |
| 18 | Window | Fluid lens 3 | Infinity | 0.3 | BK7 | 4 |
| 19 | | Conductive water | Infinity | 0.5 | 407597 | 4 |
| 20 | | Oil | 11.11111 | 0.49 | 508330 | 4 |
| 21 | Window | | Infinity | 0.3 | BK7 | 4 |
| 22 | Air gap | | Infinity | 3 | | 2.94388 |
| 23 | Lens | | 18.75 | 3.63 | SK5 | 11 |
| 24 | Air gap | | −18.75 | 0.569 | | 11 |
| 25 | Dublet | | 12.09 | 5.197 | SK5 | 11 |
| 26 | | | −12.09 | 1.026 | SF4 | 11 |
| 27 | Air gap | | 27.8 | 21.795 | | 11 |
| 28 | Lens | | 3.5 | 1.2 | BK7 | 3.5 |
| 29 | Air gap | | 3 | 0.45 | | 3.16 |
| 30 | Window | | Infinity | 1.2 | BK7 | 3.76 |
| 31 | Window | | Infinity | 0.3 | BK1 | 3.06 |
| 32 | | | Infinity | 0 | | 1.475138 |
| | Image | | Infinity | 0 | | 1.475138 |

The details for the two end zoom positions are shown in the multi-configuration table below. Configuration 1:

| | |
|---|---|
| Effective focal length | 6.19 |
| Paraxial magnification | −.0737 |

| | Curvature | Radius |
|---|---|---|
| Lens surface 7: | 0.17 | 5.882 |
| Lens surface 12 | 0.17 | 5.882 |
| Lens surface 20 | 0.049 | 20.41 |

Configuration 2:

| | |
|---|---|
| Effective focal length | 4.05 |
| Paraxial magnification | −.04899 |

| | Curvature | Radius |
|---|---|---|
| Lens surface 7: | 0.052 | 19.23 |
| Lens surface 12 | 0.052 | 19.23 |
| Lens surface 20 | 0.09 | 11.11 |

These disadvantages can be significantly reduced using a zoom lens to change both the optical power of the lens system and also the plane of optimum focus. This additional control of the operating parameters of the endoscope or imager would allow the use of a lens system with a larger numerical aperture.

Objet distance measurements can be made if the range of, or the distance to, the object is known. A fluid lens system can be used to implement a range finding system. In one embodiment, the fluid lens would be focused at a number of focus positions and the position with the best focus, as determined by any of a number of metrics, would be associated with that fluid lens position. By knowing the fluid lens drive voltage that caused the fluid lens to have an optimally focused image, and using a look-up table, the associated distance from the system for that specific fluid lens operating voltage can be determined. By knowing the range, the magnification can be calculated and thus the object width associated with a given number of pixels at the imager is known or can be deduced. In this way a system such as an endoscope or imager can calculate the width of specific object features, such the dimensions of an object.

A fluid lens variable aperture can be added to an endoscopic system. In some embodiments, the aperture would be used in the portion of the optical system that receives light and would allow the system to optimally trade light efficiency against point spread function width and depth of field. When a small aperture is used, the optical system will have a larger depth of field, but adversely the optical throughput of the system is reduced (i.e., less light gets through the system) and the point spread function (proportional to the minimal element size that can be resolved) is also reduced. In some embodiments, an endoscopic system is expected to be configured to initially have the optical system set for an optimum light throughput, and if a good image is not achieved then the aperture size could be reduced in order to extend the depth of field in an effort to observe an object that may be within the field of view.

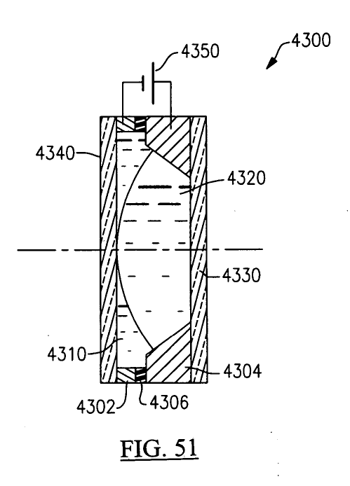

In one embodiment, a fluid lens is used as a variable aperture. FIG. 51 is a diagram 4300 showing an illustrative variable aperture comprising a fluid lens. One implementation of this use of a fluid lens involves adding a colorant to at least one of the fluids to make that fluid opaque in at least a region of an electromagnetic spectral range of interest, such as being opaque at a specified range in the visible spectrum. Voltage is applied to the lens from a power supply 4350 such that the fluid lacking the colorant that absorbs in the specified region “bottoms” against the opposite window, thereby forming a clear aperture in that spectral range of interest. An example is shown in FIG. 51, where the colorant has been added to the water component 4310 of an oil 4320/water 4310 fluid lens.

If the left window 4340 in FIG. 51 is curved such that it is effectively parallel to the curve of the water-oil interface, the liquid lens can in some instances be configured to perform as a variable filter. In such an embodiment, the oil would not bottom against the opposite window, but would produce a thickness of the water that is essentially constant as a function of radius across a portion of the window. This thickness would be varied by varying the applied voltage. The voltage-controlled thickness of the light-absorbing water would thereby determine the amount of light passing through the fluid filter. If the colorant has light absorbing characteristics in specific wavelengths, then the amplitude of the light in these wavelengths passing through the fluid filter would be varied by varying the applied voltage. The fluid lens 4300 comprises metal electrodes 4302, 4304 separated by an insulator 4306, and having a window 4330 opposite the window 4340 to allow light to pass through the fluid lens 4300.

By having more than one lens element configured as a fluid lens, for example a lens triplet, the optical aberrations present in a single element can be reduced for the assemblage of lenses and this would result in a higher quality optical image. The techniques for optimizing a triplet are well known in the lens design art. However, it is typically the case that any given lens is optimized for a given focal length system. Typically, if a lens is optimized for one combination of optical elements, it is not optimally configured when one of the lens surfaces is changed as would happen when a single fluid element is operated to change an optical parameter, such as a focal length. By adding a second fluid lens, the combination of the first lens and the second lens can be optimized to minimize total system aberrations. For different settings of the first lens, corresponding changes in the settings of the second lens can be made to obtain an optimal combination. These optimized relationships between the two fluid lens surfaces curvatures, i.e. surface optical power, and thus also the control voltages, can be contained for example in a table that is recorded in a machine readable memory. Thus for any given setting of desired system optical power, the appropriate drive voltages for the two fluid lenses can be developed, and applied in accordance with the recorded values. Where desirable or advantageous, the fineness of the table resolution may be increased through use of linear or higher order interpolation and extrapolation.

Other prior art fluid lens systems that operate using mechanical forces to control the shape and properties of a fluid lens are described in U.S. Pat. No. 4,514,048 to Rogers, which has already been incorporated herein by reference in its entirety. Additional disclosure relevant to variable focus lenses is presented in the following U.S. Pat. Nos. 2,300,251 issued Oct. 17, 1942 to Flint, No. 3,161,718 issued Dec. 15, 1964 to DeLuca, No. 3,305,294 issued Feb. 21, 1967 to Alvarez, and No. 3,583,790 issued Jun. 8, 1971 to Baker, all of which are hereby incorporated by reference herein in their entirety.

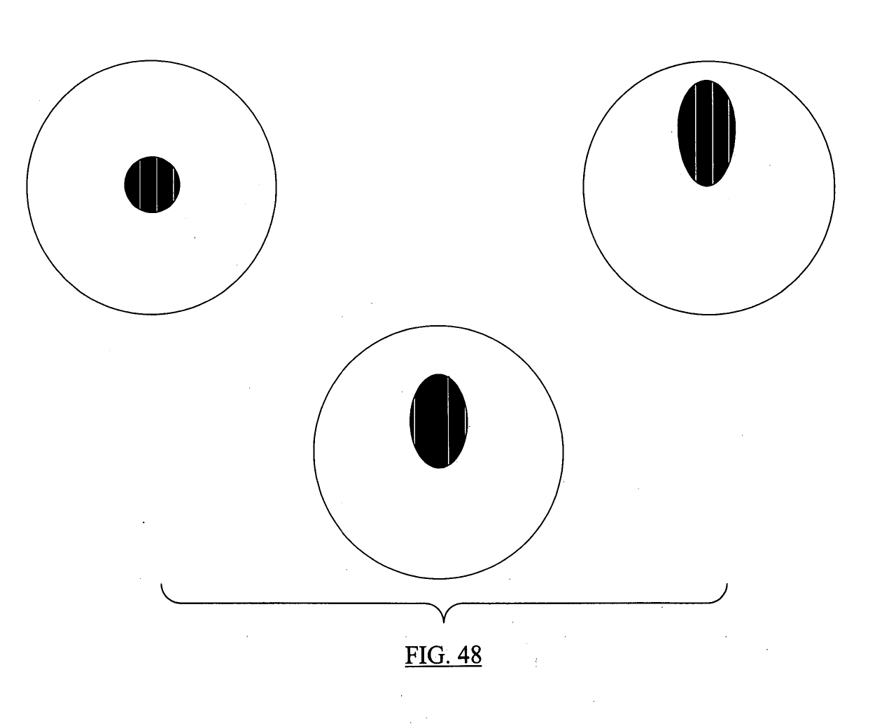

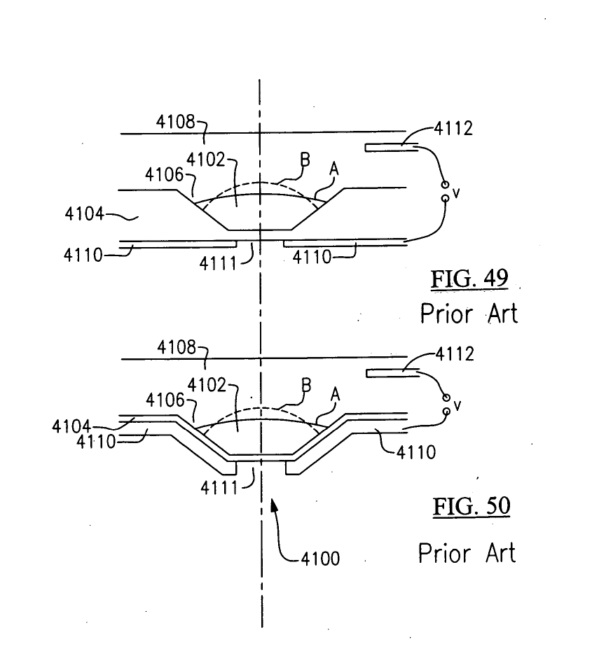

FIG. 49 and FIG. 50 are diagrams showing prior art fluid lenses that are described by Berge in U.S. Patent Application Publication US2005/0002113A1, the disclosure of which is hereby incorporated by reference herein in its entirety.

FIG. 49 shows a simplified cross-section view of a variable-focus liquid lens, formed in a dielectric enclosure 4104 filled with a conductive liquid 4108. Dielectric 4104 naturally has a low wettability with respect to conductive liquid 4108. A lower surface of a wall of enclosure 4104 includes a hollow 4106, centered around an axis O perpendicular to this wall. Hollow 4106 is a truncated cone. A drop of an isolating liquid 4102 is placed in hollow 4106. As seen previously, isolating liquid drop 4102 naturally takes a position A centered on axis O. In this embodiment, isolating liquid 4102 and conductive liquid 4108 are both transparent, non-miscible, they have different optical indexes and have substantially the same density. The dioptre formed between liquids 4108 and 4102 forms a surface of a liquid lens, the optical axis of which is axis O and the other surface of which corresponds to the contact between the drop and the bottom of the hollow. Electrode 4110, including a hole 4111 in the vicinity of axis O, is placed on the external surface of dielectric enclosure 4104. Electrode 4112 is in contact with conductive liquid 4108. Electrode 4112 may be immersed in liquid 4108, or be a conductive deposition performed on an internal wall of enclosure 4104. A voltage source (not shown) enables applying a voltage V between electrodes 4110 and 4112.

Voltage V may be increased from O volt to a maximum voltage, which depends on the used materials. When the voltage increases, isolating liquid drop 4102 deforms to reach a limiting position (designated with reference B). While drop 4102 deforms from its position A to its position B, the focus of the liquid lens varies.

It should be noted that, drop 4102 being an isolating liquid, no microdrops occur at its periphery when voltage V is high, conversely to what would occur if the drop was a conductive liquid.

The conical shape of hollow 4106 is such that, whatever the shape of drop 4102 that it contains, the curvature of its surface at any contact point between the limit of the drop and the surface is smaller than that of a tangent circle TC crossing this point. Thus, according to an aspect of the present invention, hollow 6 is such that, all along its deformation from its position A to its position B, liquid drop 4102 is continuously maintained centered on axis O. A liquid lens with a accurately fixed optical axis and with a focus varying with voltage V is thus available.

It should be noted that a hollow 4106, which ensures the continuous centering of liquid drop 4102, is relatively simple to implement.

An A.C. voltage will preferably be used for voltage V, to avoid the accumulation of electric loads across the thickness of material 4104, from the surface on which is laid drop 4102.

As an example, water charged with salts (mineral or others) or any liquid, organic or not, which is conductive or made such by addition of ionic components may be used as a conductive liquid 4108. For isolating liquid 4102, oil, an alkane or a mixture of alkanes, possibly halogenated, or any other isolating liquid non miscible with conductive liquid 4108 may be used. Dielectric wall 4104 may be a glass plate or a superposition of fluorinated polymer, epoxy resin, polyethylene. Electrode 4110 may be a metal deposition.

FIG. 50 shows a simplified cross-section view of an embodiment of a variable-focus liquid lens. In this embodiment, electrode 4110 may be a metal sheet in which hollow 4106 is formed by embossing. It may also be a metal wall in which hollow 4106 has been formed by machining, then polishing. Wall 4104 then is, for example, a thin transparent plastic film flattened against electrode 4110 and which covers hole 4111. This plastic film may for example be flattened by thermoforming.

In the example of application of FIG. 49, drop 4102 has an idle diameter of approximately 1 to 5 mm. Conductive liquid 4108 and the isolating liquid of drop 4102 being substantially of same density, drop 4102 has the shape of a spherical cap. When idle (position A), the edge of drop 4102 makes an angle of approximately 45 degrees with the surface of hollow 4106, if the latter is a cone having a 45-degree slope. In its limiting position (position B), the edge of drop 4102 makes an angle of approximately 90 degrees with the surface of enclosure 4104. The described device, using as a conductive liquid 4108 salt water having an optical index 1.35 and, as the isolating liquid of drop 4102, oil with optical index 1.45, enables obtaining approximately 30 diopters of focus variation for an applied voltage of 250 V and a dissipated electric power of a few mW. The frequency of the A.O. voltage ranges in this case between 100 and 10,000 Hz, its period being much smaller than the system response time of approximately a few hundredths of a second.

Machine-readable storage media that can be used in an endoscope according to the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media. As is known to those of skill in the machine-readable storage media arts, new media and formats for data storage are continually being devised, and any convenient, commercially available storage medium and corresponding read/write device that may become available in the future is likely to be appropriate for use, especially if it provides any of a greater storage capacity, a higher access speed, a smaller size, and a lower cost per bit of stored information. Well known older machine-readable media are also available for use under certain conditions, such as punched paper tape or cards, magnetic recording on tape or wire, optical or magnetic reading of printed characters (e.g., OCR and magnetically encoded symbols) and machine-readable symbols such as one and two dimensional bar codes.

Many functions of electrical and electronic apparatus can be implemented in hardware (for example, hard-wired logic), in software (for example, logic encoded in a program operating on a general purpose processor), and in firmware (for example, logic encoded in a non-volatile memory that is invoked for operation on a processor as required). The present invention contemplates the substitution of one implementation of hardware, firmware and software for another implementation of the equivalent functionality using a different one of hardware, firmware and software. To the extent that an implementation can be represented mathematically by a transfer function, that is, a specified response is generated at an output terminal for a specific excitation applied to an input terminal of a “black box” exhibiting the transfer function, any implementation of the transfer function, including any combination of hardware, firmware and software implementations of portions or segments of the transfer function, is contemplated herein.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of visualizing and measuring a remote object comprising the steps of:
providing an endoscope including a display, a hand set, an insertion tube, an optical system and an image sensor, wherein the optical system and the image sensor are located in a distal end of the insertion tube, said optical system comprising at least one adaptive lens;
visualizing on said display at least a portion of a portion of a remote object by placing said insertion tube proximate said object;
adjusting a focal length of said adaptive lens; and
controlling automatically a focus of said adaptive lens by sequentially capturing a plurality of images of said object, storing said plurality of images in a memory buffer, and automatically selecting an optimum image for measurement based at least in part on an image quality criteria.

2. The method of visualizing and measuring a remote object according to claim 1, wherein said adaptive lens operates based on a selected one of an electro-wetting phenomenon and an electro-capillarity phenomenon.

3. The method of visualizing and measuring a remote object according to claim 1, wherein said image quality criteria is a selected on of an edge contrast ratio, a MTF, and a surface roughness and a MTF.

4. The method of visualizing and measuring a remote object according to claim 1, wherein the endoscope is stationary.

5. The method of visualizing and measuring a remote object according to claim 1, wherein the entire endoscope is movable.

6. The method of visualizing and measuring a remote object according to claim 1, wherein said image quality criteria includes a factor based on said variable lens and a factor based on said image sensor.

7. The method of visualizing and measuring a remote object according to claim 1, wherein an illumination source is focused on an object to be inspected.

8. The method of visualizing and measuring a remote object according to claim 1, wherein illumination from an illumination source is controlled as to match a field of view.
